(19) United States
(12) Patent Application Publication
(10) Pub. No.: US 2023/0020999 A1
(43) Pub. Date: Jan. 19, 2023

(54) ANTI-CD30 ANTIBODY-DRUG CONJUGATES AND THEIR USE FOR THE TREATMENT OF HIV INFECTION

(71) Applicants: Paul Rubinstein, Chicago, IL (US); Seagen Inc., Bothell, WA (US)

(72) Inventors: Paul RUBINSTEIN, Chicago, IL (US); Nancy WHITING, Bothell, WA (US); Ryan Alan HEISER, Bothell, WA (US); Bryan Matthew GROGAN, Snohomish, WA (US); Markus Puhlmann, Kirkland, WA (US)

(21) Appl. No.: 17/772,105

(22) PCT Filed: Nov. 4, 2020
(86) PCT No.: PCT/US2020/058510

§ 371 Date: Apr. 26, 2022

Related U.S. Application Data
(60) Provisional application No. 62/930,342, filed on Nov. 4, 2019.

(51) Int. Cl. A61K 47/68; A61K 47/65; A61K 47/64; A61P 31/18

(52) U.S. Cl. CPC A61K 47/6803; A61K 47/6849; A61K 47/65; A61K 47/64; A61P 31/18

(57) ABSTRACT

The disclosure provides anti-CD30 antibody-drug conjugates and methods of using the same to increase CD4⁺ T-cell lymphocyte count or treat 1-IIV infection. The disclosure also provides articles of manufacture or kits comprising said antibody drug-conjugates that bind to CD30 for increasing CD4⁺ T-cell lymphocyte count or treating HIV infection.

### CROSS-REFERENCE TO RELATED APPLICATIONS

This application claims priority to U.S. Provisional Application No. 62/930,342 filed on Nov. 4, 2019, the contents of which is incorporated herein by reference in its entirety.

### COOPERATIVE RESEARCH AND DEVELOPMENT AGREEMENT

This invention was created in the performance of a Cooperative Research and Development Agreement with the National Institutes of Health, an Agency of the Department of Health and Human Services. The Government of the United States has certain rights in this invention.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 761682002640SEQLIST.TXT, date recorded: Oct. 29, 2020, size: 6 KB).

### TECHNICAL FIELD

The present application relates to anti-CD30 antibody-drug conjugates and methods of using the same to increase CD4⁺ T-cell lymphocyte counts or treat HIV infection.

### BACKGROUND

Human immunodeficiency virus infection and acquired immune deficiency syndrome (HIV/AIDS) is a spectrum of conditions caused by infection with the human immunodeficiency virus (HIV) Two types of HIV have been characterized: HIV-1 and HIV-2 HIV is a retrovirus that primarily infects components of the human immune system such as CD4⁺ T-cell lymphocytes, macrophages and dendritic cells. It directly and indirectly destroys CD4⁺ T cells. CD4⁺ T cells play a major role of protecting the human body of viruses and fungi, thus in there destruction, the host becomes immunodeficient, making the infected patients susceptible to infection by additional viruses (which can lead to cancers, such as lymphoma) and fungi. Even with treatment, HIV viral reservoirs persist in infected cells. T regulatory cells (Tregs) have been implicated as a possible reservoir for HIV. Tregs have been demonstrated to express CD30.

In 2016, worldwide about 36.7 million people were living with HIV and it resulted in 1 million deaths. From the time AIDS was identified in the early 1980s to 2017, the disease has caused an estimated 35 million deaths worldwide. The prevalence of people living with HIV/AIDS (PLWHA) in North America has continued to rise over time, from about 800,000 in the year 2000 to 1.06 million in 2006 and to 1.17 million in 2018. This is due, in large part, to combined anti-retroviral therapy (cART) initiated in the mid-1990s. This therapy increased longevity and, with a relatively constant yearly rate of infection, the prevalence increased accordingly. Since the implementation of cART, the incidence of AIDS-defining cancers (aggressive non-Hodgkin lymphoma (NHL), Kaposi sarcoma (KS), and cervical cancer (CC)) have declined. These 3 cancers are not caused by the HIV virus per se, but in destroying the CD4⁺ T cells, the Ebstein Barr, the HHV8, and the human papoloma virus are allowed to reactivate and induce oncogenesis, which then forms the AIDS defining malignancies, Depending on the study, AIDS-defining cancers represent 7-15%, and non-AIDS defining malignancies represent 12-27% of all deaths of PLWHA, making cancer a leading causes of death in PLWHA.

CD30 was originally identified by the monoclonal antibody Ki-1 (Schwab et al., 1982, Nature 299:65-67). This monoclonal antibody was developed against Hodgkin and Reed-Sternberg (H-RS) cells, the malignant cells of Hodgkin's lymphoma. A second monoclonal antibody, capable of binding a formalin resistant epitope different from that recognized by Ki-1, was subsequently described (Schwarting et al., 1989 Blood 74 1678-1689) The identification of four additional antibodies resulted in the creation of the CD30 cluster at the Third Leucocyte Typing Workshop in 1986 (McMichael, A, ed., 1987, Leukocyte Typing III (Oxford. Oxford University Press)). CD30 is a 120 kilodalton membrane glycoprotein (Froese et al., 1987, J. Immunol. 139: 2081-87) and a member of the TNF-receptor superfamily that has been shown to be a marker of malignant cells in Hodgkin's lymphoma and anaplastic large cell lymphoma (ALCL), a subset of non-Hodgkin's lymphoma (NHL) (Durkop et al., 1992, Cell 88:421-427). CD30 has been found to be highly expressed on the cell surface of all Hodgkin's lymphomas and the majority of ALCL (Josimovic-Alasevic et al., 1989, Eur. J. Immunol. 19:157-162).

Monoclonal antibodies specific for the CD30 antigen have been explored as vehicles for the delivery of cytostatic drugs, plant toxins and radioisotopes to cancerous cells expressing CD30 in both preclinical models and clinical studies (Engert et al., 1990, Cancer Research 50:84-88; Barth et al., 2000, Blood 95:3909-3914). In patients with Hodgkin's lymphoma, targeting of the CD30 antigen could be achieved with low doses of the anti-CD30 antibody, BerH2 (Falini et al., 1992, British Journal of Haematology 82:38-45). Yet, despite successful in vivo targeting of the malignant tumor cells, none of the patients experienced tumor regression. In a subsequent clinical trial, the toxin saporin was chemically conjugated to the BerH2 antibody and all four patients demonstrated rapid and substantial reductions in tumor mass (Falini et al., 1992, Lancet 339:1195-1196). However, in vitro studies using an antibody-drug conjugate (ADC) where the toxin dgA was conjugated to the Ki-1 antibody demonstrated only moderate efficacy when administered to patients with resistant HL in a Phase 1 clinical trial (Schnell et al., 2002, Clinical Cancer Research, 8(6):1779-1786).

Brentuximab vedotin is an antibody-drug conjugate composed of an anti-CD30 monoclonal antibody conjugated by a protease-cleavable linker to the microtubule disrupting agent, monomethyl auristatin E. Brentuximab vedotin has been approved for the treatment of classical Hodgkin lymphoma patients after failure of autologous stem cell transplant (ASCT) or after failure of at least 2 prior multi-agent chemotherapy regimens in patients who are not ASCT candidates, and as consolidation post-ASCT for Hodgkin lymphoma patients at increased risk of relapse/progression (ADCETRIS® (brentuximab vedotin) US Prescribing Information). It has also been approved for systemic anaplastic large cell lymphoma after failure of at least one prior multi-agent chemotherapy regimen.

Despite highly active combined antiretroviral therapy (cART), viral reservoirs persist in infected cells in individuals on cART. There are few therapeutic strategies to decrease the number of these persistently infected cells, and novel approaches to eliminate or reduce HIV reservoir burden and to increase CD4⁺ T-cell lymphocyte counts are urgently needed.

All references cited herein, including patent applications, patent publications, and scientific literature, are herein incorporated by reference in their entirety, as if each individual reference were specifically and individually indicated to be incorporated by reference.

### SUMMARY

Provided herein is a method of treating an HIV infection in a subject comprising administering to the subject an antibody-drug conjugate, wherein the antibody-drug conjugate comprises an anti-CD30 antibody or an antigen-binding portion thereof conjugated to a monomethyl auristatin. Also provided herein is a method of treating an HIV infection in a subject consisting essentially of administering to the subject an antibody-drug conjugate, wherein the antibody-drug conjugate comprises an anti-CD30 antibody or an antigen-binding portion thereof conjugated to a monomethyl auristatin. Also provided herein is a method of treating an HIV infection in a subject consisting of administering to the subject an antibody-drug conjugate, wherein the antibody-drug conjugate comprises an anti-CD30 antibody or an antigen-binding portion thereof conjugated to a monomethyl auristatin. In some embodiments, the HIV infection is an HIV-1 infection. In some embodiments, the subject does not have a hematologic cancer at the time of administration of the antibody-drug conjugate. In some embodiments, the subject has not had a hematologic cancer for at least 12 months prior to the administration of the antibody-drug conjugate. In some embodiments, the subject has not had a hematologic cancer for at least 24 months prior to the administration of the antibody-drug conjugate. In some embodiments, the hematologic cancer is selected from the group consisting of classical Hodgkin Lymphoma, non-Hodgkin Lymphoma, cutaneous T-cell lymphoma (CTCL), and anaplastic large cell lymphoma (ALCL). In some embodiments, the hematologic cancer is classical Hodgkin Lymphoma. In some of any of the embodiments herein, the classical Hodgkin Lymphoma is a stage IIA with bulky disease, stage IIB, stage III or stage IV classical Hodgkin Lymphoma. In some embodiments, the anaplastic large cell lymphoma (ALCL) is a systemic anaplastic large cell lymphoma (sALCL). In some embodiments, the anaplastic large cell lymphoma (ALCL) is a primary cutaneous anaplastic large cell lymphoma (pcALCL). In some embodiments, the cutaneous T-cell lymphoma (CTCL) is a mycosis fungoides (MF). In some embodiments, the mycosis fungoides (MF) is a CD30-positive mycosis fungoides (MF). In some embodiments, the anti-CD30 antibody of the antibody-drug conjugate comprises a heavy chain variable region and a light chain variable region, wherein the heavy chain variable region comprises:

(i) a CDR-H1 comprising the amino acid sequence of SEQ ID NO: 1;

(ii) a CDR-H2 comprising the amino acid sequence of SEQ ID NO: 2; and

(iii) a CDR-H3 comprising the amino acid sequence of SEQ ID NO: 3: and wherein the light chain variable region comprises:

(i) a CDR-L1 comprising the amino acid sequence of SEQ ID NO: 4;

(ii) a CDR-L2 comprising the amino acid sequence of SEQ ID NO: 5; and

(iii) a CDR-L3 comprising the amino acid sequence of SEQ ID NO: 6. In some embodiments, the anti-CD30 antibody of the antibody-drug conjugate comprises a heavy chain variable region comprising an amino acid sequence at least 85% identical to the amino acid sequence of SEQ ID NO: 7 and a light chain variable region comprising an amino acid sequence at least 85% identical to the amino acid sequence of SEQ ID NO: 8. In some embodiments, the anti-CD30 antibody of the antibody-drug conjugate comprises a heavy chain variable region comprising an amino acid sequence at least 90% identical to the amino acid sequence of SEQ ID NO: 7 and a light chain variable region comprising an amino acid sequence at least 90% identical to the amino acid sequence of SEQ ID NO: 8. In some embodiments, the anti-CD30 antibody of the antibody-drug conjugate comprises a heavy chain variable region comprising an amino acid sequence at least 95% identical to the amino acid sequence of SEQ ID NO: 7 and a light chain variable region comprising an amino acid sequence at least 95% identical to the amino acid sequence of SEQ ID NO: 8. In some embodiments, the anti-CD30 antibody of the antibody-drug conjugate comprises a heavy chain variable region comprising the amino acid sequence of SEQ ID NO: 7 and a light chain variable region comprising the amino acid sequence of SEQ ID NO: 8. In some embodiments, the anti-CD30 antibody is AC10. In some embodiments, the anti-CD30 antibody is cAC10. In some embodiments, the antibody-drug conjugate further comprises a linker between the anti-CD30 antibody or antigen-binding portion thereof and the monomethyl auristatin. In some embodiments, the linker is a cleavable peptide linker. In some embodiments, the monomethyl auristatin is monomethyl auristatin E (MMAE). In some embodiments, the monomethyl auristatin is monomethyl auristatin F (MMAF). In some embodiments, the antibody-drug conjugate is brentuximab vedotin. In some embodiments, the antibody-drug conjugate is administered at a dose ranging from of about 0.1 mg/kg to about 1.3 mg/kg of the subject's body weight. In some embodiments, the antibody-drug conjugate is administered at a dose ranging from about 0.3 mg/kg to about 0.9 mg/kg of the subject's body weight. In some embodiments, the antibody-drug conjugate is administered at a dose of about 0.3 mg/kg of the subject's body weight. In some embodiments, the antibody-drug conjugate is administered at a dose of about 0.6 mg/kg of the subject's body weight. In some embodiments, the antibody-drug conjugate is administered at a dose of about 0.9 mg/kg of the subject's body weight. In some embodiments, the antibody-drug conjugate is administered once about every 3 weeks. In some embodiments, the antibody-drug conjugate is administered once every 3 weeks. In some embodiments, the antibody-drug conjugate is administered for 6 3-week treatment cycles. In some embodiments, the antibody-drug conjugate is administered to the subject by intravenous infusion. In some embodiments, the intravenous infusion is about a 30 minute infusion. In some embodiments, the subject has a CD4 lymphocyte count of <200 cells/mm³ prior to administration of the antibody-drug conjugate. In some embodiments, the subject has plasma HIV RNA ≥1000 copies/mL prior to administration of the antibody-drug conjugate. In some embodiments, the subject has had plasma HIV RNA ≥200 copies/mL in a 3-month period prior to administration of the antibody-drug conjugate. In some embodiments, the subject has a life expectancy of greater than 9 months prior to administration of the antibody-drug conjugate. In some embodiments, the subject has an absolute neutrophil count ≥750/mm³. In some embodiments, the subject is male and has hemoglobin ≥10.5 gm/dL. In some embodiments, the subject is female and has hemoglobin ≥9.5 gm/dL. In some embodiments, the subject has serum alanine transaminase (SGPT/ALT)<2.5×upper limit of normal (ULN). In some embodiments, the subject has serum aspartate transaminase (SGOT/AST)<2.5×ULN. In some embodiments, the subject has bilirubin (total)<2.5×ULN. In some embodiments, the subject has creatinine <1.5×ULN. In some embodiments, the subject has received antiretroviral therapy (ART) for at least 24 weeks prior to administration of the antibody-drug conjugate. In some embodiments, the subject has received ART for at least 12 months prior to administration of the antibody-drug conjugate. In some embodiments, the subject has received ART for at least 24 months prior to administration of the antibody-drug conjugate. In some embodiments, the antibody-drug conjugate is administered in combination with ART. In some embodiments, the ART is a nucleoside reverse transcriptase inhibitor, non-nucleoside reverse transcriptase inhibitor, protease inhibitor, fusion inhibitor, CCR5 antagonist, integrase inhibitor, post-attachment inhibitor, or pharmacokinetic enhancer. In some embodiments, the ART comprises two or more of a nucleoside reverse transcriptase inhibitor, non-nucleoside reverse transcriptase inhibitor, protease inhibitor, fusion inhibitor, CCR5 antagonist, integrase inhibitor, post-attachment inhibitor, and pharmacokinetic enhancer. In some embodiments, the ART comprises three or more of a nucleoside reverse transcriptase inhibitor, non-nucleoside reverse transcriptase inhibitor, protease inhibitor, fusion inhibitor, CCR5 antagonist, integrase inhibitor, post-attachment inhibitor, and pharmacokinetic enhancer. In some embodiments, the ART comprises four or more of a nucleoside reverse transcriptase inhibitor, non-nucleoside reverse transcriptase inhibitor, protease inhibitor, fusion inhibitor, CCR5 antagonist, integrase inhibitor, post-attachment inhibitor, and pharmacokinetic enhancer. In some embodiments, the ART comprises one or more of abacavir, emtricitabine, lamivudine, tenofovir disoproxil fumarate, zidovudine, doravirine, efavirenz, etravirine, nevirapine, rilpivirine, atazanavir, darunavir, fosamprenavir, ritonavir, saquinavir, tipranavir, enfuvirtide, maraviroc, dolutegravir, raltegravir, ibalizumab, and cobicistat. In some embodiments, administering the antibody-drug conjugate results in a decrease in HIV viral load in the subject relative to the viral load prior to administration of the antibody-drug conjugate. In some embodiments, HIV viral load is assessed by measuring CD4⁺ T-cell-associated HIV DNA. In some embodiments, HIV viral load is assessed by measuring CD4⁺ T-cell-associated HIV RNA. In some embodiments, the subject exhibits a viral load of less than or equal to 50 copies of HIV virus particles per mL of blood plasma (<50 c/mL) after at least 24 weeks, at least 48 weeks, or at least 96 weeks after administration of the antibody-drug conjugate. In some embodiments, administering the antibody-drug conjugate results in elimination of the HIV infection in the subject. In some embodiments, administering the antibody-drug conjugate results in a decrease in the number of Treg cells relative to the number prior to the administration of the antibody-drug conjugate. In some embodiments, the Treg cells are CD4⁺. In some embodiments, the Treg cells are CD30⁺. In some embodiments, administering the antibody-drug conjugate results in a decrease in the number of memory T cells relative to the number prior to the administration of the antibody-drug conjugate. In some embodiments, the memory T cells are CD4⁺. In some embodiments, the memory T cells are CD30⁺. In some embodiments, administering the antibody-drug conjugate results in an increase in the number of CD4⁺ T cells relative to the number prior to the administration of the antibody-drug conjugate. In some embodiments, the subject is a human.

Also provided herein is a kit comprising (a) a dosage ranging from about 0.1 mg to about 500 mg of an of an antibody-drug conjugate that binds to CD30, wherein the antibody-drug conjugate comprises an anti-CD30 antibody or an antigen-binding fragment thereof conjugated to a monomethyl auristatin or a functional analog thereof or a functional derivative thereof; and (b) instructions for using the antibody drug conjugate according any of the embodiments herein.

Also provided herein is the use of an antibody-drug conjugate that binds to CD30 for the manufacture of a medicament for use in any of the embodiments herein.

Also provided herein is an antibody-drug conjugate that binds to CD30 for use in any of the embodiments herein.

Also provided herein is a method of increasing CD4⁺ T-cell lymphocyte count in a subject infected with human immunodeficiency virus (HIV) comprising administering to the subject an antibody-drug conjugate, wherein the antibody-drug conjugate comprises an anti-CD30 antibody or an antigen-binding portion thereof conjugated to a monomethyl auristatin. Also provided herein is a method of increasing CD4⁺ T-cell lymphocyte count in a subject infected with human immunodeficiency virus (HIV) consisting essentially of administering to the subject an antibody-drug conjugate, wherein the antibody-drug conjugate comprises an anti-CD30 antibody or an antigen-binding portion thereof conjugated to a monomethyl auristatin. Also provided herein is a method of increasing CD4⁺ T-cell lymphocyte count in a subject infected with human immunodeficiency virus (HIV) consisting of administering to the subject an antibody-drug conjugate, wherein the antibody-drug conjugate comprises an anti-CD30 antibody or an antigen-binding portion thereof conjugated to a monomethyl auristatin. In some embodiments, the HIV infection is an HIV-1 infection. In some embodiments, the subject has a CD4⁺ T-cell lymphocyte count of <200 cells/μL prior to administration of the antibody-drug conjugate. In some embodiments, the subject has a CD4⁺ T-cell lymphocyte count of >50 cells/μL prior to administration of the antibody-drug conjugate. In some embodiments, the subject has had a plasma HIV viral load ≤50 copies/mL for at least 6 months prior to administration of the antibody-drug conjugate. In some embodiments, the subject has had a plasma HIV viral load ≤50 copies/mL for at least 12 months prior to administration of the antibody-drug conjugate. In some embodiments, the subject has had a plasma HIV viral load 5 50 copies/mL for at least 24 months prior to administration of the antibody-drug conjugate. In some embodiments, the subject does not have a hematologic cancer at the time of administration of the antibody-drug conjugate. In some embodiments, the subject has not had a hematologic cancer for at least 12 months prior to the administration of the antibody-drug conjugate. In some embodiments, the subject has not had a hematologic cancer for at least 24 months prior to the administration of the antibody-drug conjugate. In some embodiments, the hematologic cancer is selected from the group consisting of classical Hodgkin Lymphoma, non-Hodgkin Lymphoma, cutaneous T-cell lymphoma (CTCL), and anaplastic large cell lymphoma (ALCL). In some embodiments, the hematologic cancer is classical Hodgkin Lymphoma. In some embodiments, the classical Hodgkin Lymphoma is a stage HA with bulky disease, stage IIB, stage III or stage IV classical Hodgkin Lymphoma. In some embodiments, the anaplastic large cell lymphoma (ALCL) is a systemic anaplastic large cell lymphoma (sALCL). In some embodiments, the anaplastic large cell lymphoma (ALCL) is a primary cutaneous anaplastic large cell lymphoma (pcALCL). In some embodiments, the cutaneous T-cell lymphoma (CTCL) is a mycosis fungoides (MF). In some embodiments, the mycosis fungoides (MF) is a CD30-positive mycosis fungoides (MF). In some embodiments, the anti-CD30 antibody of the antibody-drug conjugate comprises a heavy chain variable region and a light chain variable region, wherein the heavy chain variable region comprises:

(i) a CDR-H1 comprising the amino acid sequence of SEQ ID NO: 1;

(ii) a CDR-H2 comprising the amino acid sequence of SEQ ID NO: 2; and

(iii) a CDR-H3 comprising the amino acid sequence of SEQ ID NO: 3; and wherein the light chain variable region comprises:

(i) a CDR-L1 comprising the amino acid sequence of SEQ ID NO: 4;

(ii) a CDR-L2 comprising the amino acid sequence of SEQ ID NO: 5; and

(iii) a CDR-L3 comprising the amino acid sequence of SEQ ID NO: 6. In some embodiments, the anti-CD30 antibody of the antibody-drug conjugate comprises a heavy chain variable region comprising an amino acid sequence at least 85% identical to the amino acid sequence of SEQ ID NO: 7 and a light chain variable region comprising an amino acid sequence at least 85% identical to the amino acid sequence of SEQ ID NO: 8. In some embodiments, the anti-CD30 antibody of the antibody-drug conjugate comprises a heavy chain variable region comprising an amino acid sequence at least 90% identical to the amino acid sequence of SEQ ID NO: 7 and a light chain variable region comprising an amino acid sequence at least 90% identical to the amino acid sequence of SEQ ID NO: 8. In some embodiments, the anti-CD30 antibody of the antibody-drug conjugate comprises a heavy chain variable region comprising an amino acid sequence at least 95% identical to the amino acid sequence of SEQ ID NO: 7 and a light chain variable region comprising an amino acid sequence at least 95% identical to the amino acid sequence of SEQ ID NO: 8. In some embodiments, the anti-CD30 antibody of the antibody-drug conjugate comprises a heavy chain variable region comprising the amino acid sequence of SEQ ID NO: 7 and a light chain variable region comprising the amino acid sequence of SEQ ID NO: 8. In some embodiments, the anti-CD30 antibody is AC10. In some embodiments, the anti-CD30 antibody is cAC10. In some embodiments, the antibody-drug conjugate further comprises a linker between the anti-CD30 antibody or antigen-binding portion thereof and the monomethyl auristatin. In some embodiments, the linker is a cleavable peptide linker. In some embodiments, the cleavable peptide linker has a formula: -MC-vc-PAB-. In some embodiments, the monomethyl auristatin is monomethyl auristatin E (MMAE). In some embodiments, the monomethyl auristatin is monomethyl auristatin F (MMAF). In some embodiments, the antibody-drug conjugate is brentuximab vedotin. In some embodiments, the antibody-drug conjugate is administered at a dose ranging from of about 1.2 mg/kg of the subject's body weight. In some embodiments, the antibody-drug conjugate is administered at a dose ranging from of 1.2 mg/kg of the subject's body weight. In some embodiments, the antibody-drug conjugate is administered at a dose of about 0.9 mg/kg of the subject's body weight. In some embodiments, the antibody-drug conjugate is administered at a dose of 0.9 mg/kg of the subject's body weight. In some embodiments, the antibody-drug conjugate is administered once about every 2 weeks. In some embodiments, the antibody-drug conjugate is administered once every 2 weeks. In some embodiments, the antibody-drug conjugate is administered for four 2-week treatment cycles. In some embodiments, the antibody-drug conjugate is administered to the subject by intravenous infusion. In some embodiments, the intravenous infusion is about a 30 minute infusion. In some embodiments, the subject has a life expectancy of greater than 9 months prior to administration of the antibody-drug conjugate. In some embodiments, the subject has received antiretroviral therapy (ART) for at least 24 weeks prior to administration of the antibody-drug conjugate. In some embodiments, the subject has received ART for at least 12 months prior to administration of the antibody-drug conjugate. In some embodiments, the subject has received ART for at least 24 months prior to administration of the antibody-drug conjugate. In some embodiments, the antibody-drug conjugate is administered in combination with ART. In some embodiments, the ART is a nucleoside reverse transcriptase inhibitor, non-nucleoside reverse transcriptase inhibitor, protease inhibitor, fusion inhibitor, CCR5 antagonist, integrase inhibitor, post-attachment inhibitor, or pharmacokinetic enhancer. In some embodiments, the ART comprises two or more of a nucleoside reverse transcriptase inhibitor, non-nucleoside reverse transcriptase inhibitor, protease inhibitor, fusion inhibitor, CCR5 antagonist, integrase inhibitor, post-attachment inhibitor, and pharmacokinetic enhancer. In some embodiments, the ART comprises three or more of a nucleoside reverse transcriptase inhibitor, non-nucleoside reverse transcriptase inhibitor, protease inhibitor, fusion inhibitor, CCR5 antagonist, integrase inhibitor, post-attachment inhibitor, and pharmacokinetic enhancer. In some embodiments, the ART comprises four or more of a nucleoside reverse transcriptase inhibitor, non-nucleoside reverse transcriptase inhibitor, protease inhibitor, fusion inhibitor, CCR5 antagonist, integrase inhibitor, post-attachment inhibitor, and pharmacokinetic enhancer. In some embodiments, the ART comprises one or more of abacavir, emtricitabine, lamivudine, tenofovir disoproxil fumarate, zidovudine, doravirine, efavirenz, etravirine, nevirapine, rilpivirine, atazanavir, darunavir, fosamprenavir, ritonavir, saquinavir, tipranavir, enfuvirtide, maraviroc, dolutegravir, raltegravir, ibalizumab, and cobicistat. In some embodiments, the ART does not comprise a strong CYP3A4 inhibitor. In some embodiments, the ART does not comprise a strong P-gp inhibitor. In some embodiments, administering the antibody-drug conjugate results an increase in the CD4⁺ T-cell lymphocyte count in the subject to above 200 cells/μL. In some embodiments, administering the antibody-drug conjugate results in an increase in the CD4⁺ T-cell lymphocyte count by at least 50 cells/μL relative to the CD4⁺ T-cell lymphocyte count prior to administration. In some embodiments, administering the antibody-drug conjugate results an increase in the CD8⁺ T-cell lymphocyte count in the subject relative to the CD8⁺ T-cell lymphocyte count prior to administration. In some embodiments, administering the antibody-drug conjugate results in a decrease in the number of Treg cells relative to the number prior to the administration of the antibody-drug conjugate. In some embodiments, the Treg cells are CD4⁺. In some embodiments, the Treg cells are CD30⁺. In some embodiments, administering the antibody-drug conjugate results in a decrease in the number of memory T cells relative to the number prior to the administration of the antibody-drug conjugate. In some embodiments, the memory T cells are CD4⁺. In some embodiments, the memory T cells are CD30⁺. In some embodiments, the subject has not been administered the antibody-drug conjugate prior to the administration to increase CD4⁺ T-cell lymphocyte count in the subject. In some embodiments, the subject is a human.

Also provided herein is a kit comprising (a) a dosage ranging from about 0.1 mg to about 500 mg of an of an antibody-drug conjugate that binds to CD30, wherein the antibody-drug conjugate comprises an anti-CD30 antibody or an antigen-binding fragment thereof conjugated to a monomethyl auristatin or a functional analog thereof or a functional derivative thereof; and (b) instructions for using the antibody drug conjugate according to any of the embodiments herein.

Also provided herein is the use of an antibody-drug conjugate that binds to CD30 for the manufacture of a medicament for use in any of the embodiments herein.

Also provided herein is an antibody-drug conjugate that binds to CD30 for use in any of the embodiments herein.

### BRIEF DESCRIPTION OF THE DRAWINGS

FIG. 1A-1B are graphs showing the effect of various concentrations of brentuximab vedotin (BV) on the total number of viable cells in various T cell populations (FIG. 1A) or the number of CD30⁺ viable cells in various T cell populations (FIG. 1B). The various T cell populations are displayed as Treg (⊙), naïve CD4 (♦), memory CD4 (▴), naïve CD8 (▾), and memory CD8 (▪). Data are represented as viable cell counts relative to untreated control.

FIG. 2A-2B are graphs showing CD30 and CD30L expression in various T cell populations over time as monitored by flow cytometry. FIG. 2A shows a representative flow cytometry plot over a 3-day activation time-course. FIG. 2B shows the percent of each T cell population that are CD30⁺ over time. The various T cell populations are displayed as Treg (⊙), naïve CD4 (♦), memory CD4 (▴), naïve CD8 (▾), and memory CD8 (▪).

FIG. 3A-3B are graphs showing rhodamine 123 efflux over time in various T cell populations as measured by flow cytometry. FIG. 3A shows rhodamine efflux at 37° C. in Treg, naïve CD4 (CD4 TN), memory CD4 (CD4 TMEM), naïve CD8 (CD8 TM), and memory CD8 (CD8 TMEM) T cells. FIG. 3B shows rhodamine efflux over a 3-hour time course in various T cell populations. The various T cell populations are displayed as Treg (⊙), naïve CD4 (♦), memory CD4 (▴), naïve CD8 (▾), and memory CD8 (▪).

FIG. 4 is a graph showing the effects of treating various T cell populations with a titration of free monomethyl auristatin E (MMAE) over the course of 4 days. The various T cell populations are displayed as Treg (└), naïve CD4 (♦), memory CD4 (▴), naïve CD8 (▾), and memory CD8 (▪).

### DETAILED DESCRIPTION

### I. Definitions

In order that the present disclosure can be more readily understood, certain terms are first defined. As used in this application, except as otherwise expressly provided herein, each of the following terms shall have the meaning set forth below. Additional definitions are set forth throughout the application.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C: A and C; A and B: B and C; A (alone); B (alone); and C (alone).

It is understood that aspects and embodiments of the invention described herein include “comprising,” “consisting,” and “consisting essentially of” aspects and embodiments.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Systéme International de Unites (SI) accepted form. Numeric ranges are inclusive of the numbers defining the range. The headings provided herein are not limitations of the various aspects of the disclosure, which can be had by reference to the specification as a whole. Accordingly, the terms defined immediately below are more fully defined by reference to the specification in its entirety.

“CD30” or “TNFRSF8” refers to a receptor that is a member of the tumor necrosis factor receptor superfamily. CD30 is a transmembrane glycoprotein expressed on activated CD4⁺ and CD8′ T cells and B cells, and virally-infected lymphocytes. CD30 interacts with TRAF2 and TRAF3 to mediate signal transduction that leads to activation of NF-κB. CD30 acts as a positive regulator of apoptosis, and it has been shown to limit the proliferative potential of auto-reactive CD8 effector T cells. CD30 is also expressed by various forms of lymphoma, including Hodgkin lymphoma (CD30 is expressed by Reed-Sternberg cells) and non-Hodgkin lymphoma (e.g., diffuse large B-cell lymphoma (DLBCL), peripheral T-cell lymphoma (PTCL), and cutaneous T-cell lymphoma (CTCL).

The terms “Treg” or “regulatory T cell” refer to CD4⁺ T cells that suppresses CD4⁺ CD25⁺ and CD8⁺ T cell proliferation and/or effector function, or that otherwise down-modulate an immune response. Notably. Treg may down-regulate immune responses mediated by Natural Killer cells, Natural Killer T cells as well as other immune cells.

The terms “regulatory T cell function” or “a function of Treg” are used interchangeably to refer to any biological function of a Treg that results in a reduction in CD4⁺ CD25⁺ or CD8⁺ T cell proliferation or a reduction in an effector T cell-mediated immune response Treg function can be measured via techniques established in the art. Non-limiting examples of useful in vitro assays for measuring Treg function include Transwell suppression assays as well as in vitro assays in which the target conventional T cells (Tconv) and Tregs purified from human peripheral blood or umbilical cord blood (or murine spleens or lymph nodes) are optionally activated by anti-CD3⁺ anti-CD28 coated beads (or antigen-presenting cells (APCs) such as, e.g, irradiated splenocytes or purified dendritic cells (DCs) or irradiated PBMCs) followed by in vitro detection of conventional T cell proliferation (e.g., by measuring incorporation of radioactive nucleouides (such as, e.g, [H]-thymidine) or fluorescent nucleotides, or by Cayman Chemical MTT Cell Proliferation Assay Kit, or by monitoring the dilution of a green fluorochrome ester CFSE or Seminaphtharhodafluor (SNARF-1) dye by flow cytometry). Other common assays measure T cell cytokine responses Useful in vivo assays of Treg function include assays in animal models of diseases in which Tregs play an important role, including, e.g., (1) homeostasis model (using naïve homeostatically expanding CD4⁺ T cells as target cells that are primarily suppressed by Tregs), (2) inflammatory bowel disease (IBD) recovery model (using Th1 T cells (Th17) as target cells that are primarily suppressed by Tregs), (3) experimental autoimmune encephalomyelitis (EAE) model (using Thl 7 and Thl T cells as target cells that are primarily suppressed by Tregs), (4) B16 melanoma model (suppression of antitumor immunity) (using CD8⁺ T cells as target cells that are primarily suppressed by Tregs), (5) suppression of colon inflammation in adoptive transfer colitis where naïve CD4CD45RBM Tconv cells are transferred into RagV mice, and (6) Foxp3 rescue model (using lymphocytes as target cells that are primarily suppressed by Tregs). According to one protocol, all of the models require mice for donor T cell populations as well as Ragl^{-/-} or Foxp3 mice for recipients. For more details on various useful assays see, e.g., Collison and Vignali, In Vitro Treg Suppression Assays, Chapter 2 in Regulatory T Cells: Methods and Protocols, Methods in Molecular Biology, Kassiotis and Liston eds., Springer, 2011, 707:21-37; Workman et al, In Vivo Treg Suppression Assays, Chapter 9 in Regulatory T Cells: Methods and Protocols, Methods in Molecular Biology, Kassiotis and Liston eds., Springer, 2011, 119-156; Takahashi et al, Int. Immunol, 1998, 10 1969-1980; Thornton et al, J. Exp Med., 1998, 188287-296: Collison et al, J Immunol, 2009, 182:6121-6128; Thornton and Shevach, J Exp Med, 1998, 188:287-296: Asseman et al, J. Exp. Med, 1999, 190:995-1004: Dieckmann et al, J. Exp Med., 2001, 193: 1303-1310; Belkaid, Nature Reviews, 2007, 7:875-888; Tang and Bluestone, Nature Immunology, 2008, 9:239-244; Bettini and Vignali, Curr. Opin. Immunol, 2009, 21 612-618: Dannull et al, J Clin Invest, 2005, 115(12):3623-33; Tsaknaridis, et al, J Neurosci Res., 2003, 74:296-308.

The term “immunotherapy” refers to the treatment of a subject afflicted with, at risk of contracting, or suffering a recurrence of a disease by a method comprising inducing, enhancing, suppressing, or otherwise modifying an immune response.

“Administering” refers to the physical introduction of a therapeutic agent to a subject, using any of the various methods and delivery systems known to those skilled in the art. Exemplary routes of administration include intravenous, intramuscular, subcutaneous, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. A therapeutic agent can be administered via a non-parenteral route, or orally. Other non-parenteral routes include a topical, epidermal or mucosal route of administration, for example, intranasally, vaginally, rectally, sublingually or topically. Administering can also be performed, for example, once, a plurality of times, and/or over one or more extended periods.

The terms “baseline” or “baseline value” used interchangeably herein can refer to a measurement or characterization of a symptom before the administration of the therapy (e.g., an anti-CD30 antibody-drug conjugate as described herein) or at the beginning of administration of the therapy. The baseline value can be compared to a reference value in order to determine the reduction or improvement of a symptom of a CD30-associated disease contemplated herein (e.g., HIV infection). The terms “reference” or “reference value” used interchangeably herein can refer to a measurement or characterization of a symptom after administration of the therapy (e.g., an anti-CD30 antibody-drug conjugate as described herein). The reference value can be measured one or more times during a dosage regimen or treatment cycle or at the completion of the dosage regimen or treatment cycle. A “reference value” can be an absolute value; a relative value; a value that has an upper and/or lower limit; a range of values; an average value; a median value: a mean value; or a value as compared to a baseline value.

Similarly, a “baseline value” can be an absolute value; a relative value; a value that has an upper and/or lower limit; a range of values; an average value; a median value; a mean value; or a value as compared to a reference value. The reference value and/or baseline value can be obtained from one individual, from two different individuals or from a group of individuals (e.g., a group of two, three, four, five or more individuals).

The term “monotherapy” as used herein means that the anti-CD30 antibody-drug conjugate is the only anti-HIV agent administered to the subject during the treatment cycle. Other therapeutic agents, however, can be administered to the subject. For example, anti-inflammatory agents or other agents administered to a subject with HIV to treat symptoms associated with HIV infection, but not the underlying HIV infection itself, including, for example inflammation, pain, weight loss, and general malaise, can be administered during the period of monotherapy.

An “adverse event” (AE) as used herein is any unfavorable and generally unintended or undesirable sign (including an abnormal laboratory finding), symptom, or disease associated with the use of a medical treatment. A medical treatment can have one or more associated AEs and each AE can have the same or different level of severity. Reference to methods capable of “altering adverse events” means a treatment regime that decreases the incidence and/or severity of one or more AEs associated with the use of a different treatment regime.

A “serious adverse event” or “SAE” as used herein is an adverse event that meets one of the following criteria:
Is fatal or life-threatening (as used in the definition of a serious adverse event, “life-threatening” refers to an event in which the patient was at risk of death at the time of the event; it does not refer to an event which hypothetically might have caused death if it was more severe.
Results in persistent or significant disability/incapacity
Constitutes a congenital anomaly/birth defect
Is medically significant, i.e., defined as an event that jeopardizes the patient or may require medical or surgical intervention to prevent one of the outcomes listed above. Medical and scientific judgment must be exercised in deciding whether an AE is “medically significant”
Requires inpatient hospitalization or prolongation of existing hospitalization, excluding the following: 1) routine treatment or monitoring of the underlying disease, not associated with any deterioration in condition; 2) elective or pre-planned treatment for a pre-existing condition that is unrelated to the indication under study and has not worsened since signing the informed consent; and 3) social reasons and respite care in the absence of any deterioration in the patient's general condition.

The term “immunoglobulin” refers to a class of structurally related glycoproteins consisting of two pairs of polypeptide chains, one pair of light (L) low molecular weight chains and one pair of heavy (H) chains, all four inter-connected by disulfide bonds. The structure of immunoglobulins has been well characterized. See for instance Fundamental Immunology Ch. 7 (Paul, W., ed., 2nd ed. Raven Press, N.Y. (1989)). Briefly, each heavy chain typically is comprised of a heavy chain variable region (abbreviated herein as V_{H} or VH) and a heavy chain constant region (C_{H} or CH). The heavy chain constant region typically is comprised of three domains, C_{H}1, C_{H}2, and C_{H}3. The heavy chains are generally inter-connected via disulfide bonds in the so-called “hinge region.” Each light chain typically is comprised of a light chain variable region (abbreviated herein as V_{L} or VL) and a light chain constant region (C_{L} or CL). The light chain constant region typically is comprised of one domain, C_{L}. The CL can be of c (kappa) or X (lambda) isotype. The terms “constant domain” and “constant region” are used interchangeably herein. An immunoglobulin can derive from any of the commonly known isotypes, including but not limited to IgA, secretory IgA, IgG, and IgM. IgG subclasses are also well known to those in the art and include but are not limited to human IgG1, IgG2, IgG3 and IgG4. “Isotype” refers to the antibody class or subclass (e.g., IgM or IgG1) that is encoded by the heavy chain constant region genes.

The term “variable region” or “variable domain” refers to the domain of an antibody heavy or light chain that is involved in binding the antibody to antigen. The variable regions of the heavy chain and light chain (V_{H} and V_{L}, respectively) of a native antibody may be further subdivided into regions of hypervariability (or hypervariable regions, which may be hypervariable in sequence and/or form of structurally defined loops), also termed complementarity-determining regions (CDRs), interspersed with regions that are more conserved, termed framework regions (FRs). The terms “complementarity determining regions” and “CDRs,” synonymous with “hypervariable regions” or “HVRs” are known in the art to refer to non-contiguous sequences of amino acids within antibody variable regions, which confer antigen specificity and/or binding affinity. In general, there are three CDRs in each heavy chain variable region (CDR-H1, CDR-H2, CDR-H3) and three CDRs in each light chain variable region (CDR-L1, CDR-L2, CDR-L3). “Framework regions” and “FR” are known in the art to refer to the non-CDR portions of the variable regions of the heavy and light chains. In general, there are four FRs in each full-length heavy chain variable region (FR-H1, FR-H2, FR-H3, and FR-H4), and four FRs in each full-length light chain variable region (FR-L1, FR-L2, FR-L3, and FR-L4). Within each V_{H} and V_{L}, three CDRs and four FRs are typically arranged from amino-terminus to carboxy-terminus in the following order: FR1, CDR1, FR2, CDR2, FR3, CDR3, FR4 (See also Chothia and Lesk J. Mot. Biol., 195, 901-917 (1987)).

The term “antibody” (Ab) in the context of the present invention refers to an immunoglobulin molecule, a fragment of an immunoglobulin molecule, or a derivative of either thereof, which has the ability to specifically bind to an antigen under typical physiological conditions with a half-life of significant periods of time, such as at least about 30 min, at least about 45 min, at least about one hour (h), at least about two hours, at least about four hours, at least about eight hours, at least about 12 hours (h), about 24 hours or more, about 48 hours or more, about three, four, five, six, seven or more days, etc., or any other relevant functionally-defined period (such as a time sufficient to induce, promote, enhance, and/or modulate a physiological response associated with antibody binding to the antigen and/or time sufficient for the antibody to recruit an effector activity). The variable regions of the heavy and light chains of the immunoglobulin molecule contain a binding domain that interacts with an antigen. The constant regions of the antibodies (Abs) may mediate the binding of the immunoglobulin to host tissues or factors, including various cells of the immune system (such as effector cells) and components of the complement system such as C1q, the first component in the classical pathway of complement activation. An antibody may also be a bispecific antibody, diabody, multispecific antibody or similar molecule.

The term “monoclonal antibody” as used herein refers to a preparation of antibody molecules that are recombinantly produced with a single primary amino acid sequence. A monoclonal antibody composition displays a single binding specificity and affinity for a particular epitope. Accordingly, the term “human monoclonal antibody” refers to antibodies displaying a single binding specificity which have variable and constant regions derived from human germline immunoglobulin sequences. The human monoclonal antibodies may be generated by a hybridoma which includes a B cell obtained from a transgenic or transchromosomal non-human animal, such as a transgenic mouse, having a genome comprising a human heavy chain transgene and a light chain transgene, fused to an immortalized cell.

An “isolated antibody” refers to an antibody that is substantially free of other antibodies having different antigenic specificities (e.g., an isolated antibody that binds specifically to CD30 is substantially free of antibodies that bind specifically to antigens other than CD30). An isolated antibody that binds specifically to CD30 can, however, have cross-reactivity to other antigens, such as CD30 molecules from different species. Moreover, an isolated antibody can be substantially free of other cellular material and/or chemicals. In one embodiment, an isolated antibody includes an antibody conjugate attached to another agent (e.g., small molecule drug). In some embodiments, an isolated anti-CD30 antibody includes a conjugate of an anti-CD30 antibody with a small molecule drug (e.g., MMAE or MMAF).

A “human antibody” (HuMAb) refers to an antibody having variable regions in which both the FRs and CDRs are derived from human germline immunoglobulin sequences. Furthermore, if the antibody contains a constant region, the constant region also is derived from human germline immunoglobulin sequences. The human antibodies of the disclosure can include amino acid residues not encoded by human germline immunoglobulin sequences (e.g., mutations introduced by random or site-specific mutagenesis in vitro or by somatic mutation in vivo). However, the term “human antibody,” as used herein, is not intended to include antibodies in which CDR sequences derived from the germline of another mammalian species, such as a mouse, have been grafted onto human framework sequences. The terms “human antibodies” and “fully human antibodies” and are used synonymously.

The term “humanized antibody” as used herein, refers to a genetically engineered non-human antibody, which contains human antibody constant domains and non-human variable domains modified to contain a high level of sequence homology to human variable domains. This can be achieved by grafting of the six non-human antibody complementarity-determining regions (CDRs), which together form the antigen binding site, onto a homologous human acceptor framework region (FR) (see WO92/22653 and EP0629240). In order to fully reconstitute the binding affinity and specificity of the parental antibody, the substitution of framework residues from the parental antibody (i.e. the non-human antibody) into the human framework regions (back-mutations) may be required. Structural homology modeling may help to identify the amino acid residues in the framework regions that are important for the binding properties of the antibody. Thus, a humanized antibody may comprise non-human CDR sequences, primarily human framework regions optionally comprising one or more amino acid back-mutations to the non-human amino acid sequence, and fully human constant regions. Optionally, additional amino acid modifications, which are not necessarily back-mutations, may be applied to obtain a humanized antibody with preferred characteristics, such as affinity and biochemical properties.

The term “chimeric antibody” as used herein, refers to an antibody wherein the variable region is derived from a non-human species (e.g. derived from rodents) and the constant region is derived from a different species, such as human. Chimeric antibodies may be generated by antibody engineering. “Antibody engineering” is a term used generic for different kinds of modifications of antibodies, and which is a well-known process for the skilled person. In particular, a chimeric antibody may be generated by using standard DNA techniques as described in Sambrook et al., 1989, Molecular Cloning: A laboratory Manual, New York: Cold Spring Harbor Laboratory Press, Ch. 15. Thus, the chimeric antibody may be a genetically or an enzymatically engineered recombinant antibody. It is within the knowledge of the skilled person to generate a chimeric antibody, and thus, generation of the chimeric antibody according to the present invention may be performed by other methods than described herein. Chimeric monoclonal antibodies for therapeutic applications are developed to reduce antibody immunogenicity. They may typically contain non-human (e.g. murine) variable regions, which are specific for the antigen of interest, and human constant antibody heavy and light chain domains. The terms “variable region” or “variable domains” as used in the context of chimeric antibodies, refers to a region which comprises the CDRs and framework regions of both the heavy and light chains of the immunoglobulin.

An “anti-antigen antibody” refers to an antibody that binds to the antigen. For example, an anti-CD30 antibody is an antibody that binds to the antigen CD30.

An “antigen-binding portion” or antigen-binding fragment” of an antibody refers to one or more fragments of an antibody that retain the ability to bind specifically to the antigen bound by the whole antibody. Examples of antibody fragments (e.g., antigen-binding fragment) include but are not limited to Fv, Fab, Fab′, Fab′-SH, F(ab′)₂; diabodies; linear antibodies; single-chain antibody molecules (e.g. scFv); and multispecific antibodies formed from antibody fragments. Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-combining sites and is still capable of cross-linking antigen.

“Percent (%) sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For example, the % sequence identity of a given amino acid sequence A to, with, or against a given amino acid sequence B (which can alternatively be phrased as a given amino acid sequence A that has or comprises a certain % sequence identity to, with, or against a given amino acid sequence B) is calculated as follows:

100 times the fraction X/Y

where X is the number of amino acid residues scored as identical matches by the sequence in that program's alignment of A and B, and where Y is the total number of amino acid residues in B. It will be appreciated that where the length of amino acid sequence A is not equal to the length of amino acid sequence B, the % sequence identity of A to B will not equal the % sequence identity of B to A.

As used herein, the terms “binding”, “binds” or “specifically binds” in the context of the binding of an antibody to a pre-determined antigen typically is a binding with an affinity corresponding to a K_{D} of about 10⁻⁶ M or less, e.g. 10⁻⁷ M or less, such as about 10⁻⁸ M or less, such as about 10⁻⁹ M or less, about 10⁻¹⁰ M or less, or about 10⁻¹¹ M or even less when determined by for instance BioLayer Interferometry (BLI) technology in a Octet HTX instrument using the antibody as the ligand and the antigen as the analyte, and wherein the antibody binds to the predetermined antigen with an affinity corresponding to a K_{D} that is at least ten-fold lower, such as at least 100-fold lower, for instance at least 1,000-fold lower, such as at least 10,000-fold lower, for instance at least 100,000-fold lower than its K_{D} of binding to a non-specific antigen (e.g., BSA, casein) other than the predetermined antigen or a closely related antigen. The amount with which the K_{D} of binding is lower is dependent on the K_{D} of the antibody, so that when the K_{D} of the antibody is very low, then the amount with which the K_{D} of binding to the antigen is lower than the K_{D} of binding to a non-specific antigen may be at least 10,000-fold (that is, the antibody is highly specific).

The term “K_{D}” (M), as used herein, refers to the dissociation equilibrium constant of a particular antibody-antigen interaction. Affinity, as used herein, and K_{D} are inversely related, that is that higher affinity is intended to refer to lower K_{D}, and lower affinity is intended to refer to higher K_{D}.

The term “ADC” refers to an antibody-drug conjugate, which in the context of the present invention refers to an anti-CD30 antibody, which is coupled to a drug moiety (e.g., MMAE or MMAF) as described in the present application.

The abbreviations “vc” and “val-cit” refer to the dipeptide valine-citrulline.

The abbreviation “PAB” refers to the self-immolative spacer:

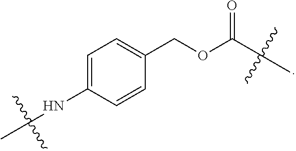 [SMILES: *Nc1ccc(COC(*)=O)cc1]

The abbreviation “MC” refers to the stretcher maleimidocaproyl:

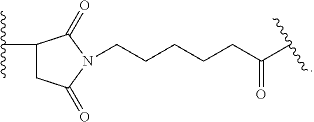 [SMILES: *C(=O)CCCCCN1C(=O)CC(*)C1=O]

The term “Ab-MC-vc-PAB-MMAE” refers to an antibody conjugated to the drug MMAE through a MC-vc-PAB linker.

The term “cAC10-MC-vc-PAB-MMAE” refers to a chimeric AC10 antibody conjugated to the drug MMAE through a MC-vc-PAB linker.

An “anti-CD30 vc-PAB-MMAE antibody-drug conjugate” refers to an anti-CD30 antibody conjugated to the drug MMAE via a linker comprising the dipeptide valine citrulline and the self-immolative spacer PAB as shown in Formula (I) of U.S. Pat. No. 9,211,319.

“Treatment” or “therapy” of a subject or “treating” a subject refers to any type of intervention or process performed on, or the administration of an active agent to, the subject with the objective of reversing, alleviating, ameliorating, inhibiting, slowing down, or preventing the onset, progression, development, severity, or recurrence of a symptom, complication, condition, or biochemical indicia associated with a disease.

A “subject” includes any human or non-human animal. The term “non-human animal” includes, but is not limited to, vertebrates such as non-human primates, sheep, dogs, and rodents such as mice, rats, and guinea pigs. In some embodiments, the subject is a human. The terms “subject” and “patient” and “individual” are used interchangeably herein.

An “effective amount” or “therapeutically effective amount” or “therapeutically effective dosage” of a drug or therapeutic agent is any amount of the drug that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

As used herein, “subtherapeutic dose” means a dose of a therapeutic compound (e.g., an antibody) that is lower than the usual or typical dose of the therapeutic compound when administered alone for the treatment of a disease.

A therapeutically effective amount of a drug includes a “prophylactically effective amount,” which is any amount of the drug that, when administered alone or in combination with an another agent to a subject at risk of developing a disease or of suffering a recurrence of disease, inhibits the development or recurrence of the disease. In some embodiments, the prophylactically effective amount prevents the development or recurrence of the disease entirely. “Inhibiting” the development or recurrence of a disease means either lessening the likelihood of the disease's development or recurrence, or preventing the development or recurrence of the disease entirely.

The term “weight-based dose”, as referred to herein, means that a dose administered to a patient is calculated based on the weight of the patient. For example, when a patient with 60 kg body weight requires 3 mg/kg of an anti-CD30 antibody, one can calculate and use the appropriate amount of the anti-CD30 antibody (i.e., 180 mg) for administration.

The use of the term “flat dose” with regard to the methods and dosages of the disclosure means a dose that is administered to a patient without regard for the weight or body surface area (BSA) of the patient. The flat dose is therefore not provided as a mg/kg dose, but rather as an absolute amount of the agent (e.g., the anti-CD30 antibody). For example, a 60 kg person and a 100 kg person would receive the same dose of an antibody (e.g., 240 mg of an anti-CD30 antibody).

The phrase “pharmaceutically acceptable” indicates that the substance or composition must be compatible chemically and/or toxicologically, with the other ingredients comprising a formulation, and/or the mammal being treated therewith.

The phrase “pharmaceutically acceptable salt” as used herein, refers to pharmaceutically acceptable organic or inorganic salts of a compound of the invention. Exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate “mesylate”, ethanesulfonate, benzenesulfonate, /?-toluenesulfonate, pamoate (i.e., T,Γ-methylene-bis-(2-hydroxy-3-naphthoate)) salts, alkali metal (e.g, sodium and potassium) salts, alkaline earth metal (e.g, magnesium) salts, and ammonium salts. A pharmaceutically acceptable salt may involve the inclusion of another molecule such as an acetate ion, a succinate ion or other counter ion. The counter ion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions Hence, a pharmaceutically acceptable salt can have one or more charged atoms and/or one or more counter ion.

The use of the alternative (e.g., “or”) should be understood to mean either one, both, or any combination thereof of the alternatives. As used herein, the indefinite articles “a” or “an” should be understood to refer to “one or more” of any recited or enumerated component.

The terms “about” or “comprising essentially of” refer to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” can mean within 1 or more than 1 standard deviation per the practice in the art. Alternatively, “about” or “comprising essentially of” can mean a range of up to 20%. Furthermore, particularly with respect to biological systems or processes, the terms can mean up to an order of magnitude or up to 5-fold of a value. When particular values or compositions are provided in the application and claims, unless otherwise stated, the meaning of “about” or “comprising essentially of” should be assumed to be within an acceptable error range for that particular value or composition.

The terms “once about every week,” “once about every two weeks,” “once about every three weeks” or any other similar dosing interval terms as used herein mean approximate numbers. “Once about every week” can include every seven days f one day, i.e., every six days to every eight days. “Once about every two weeks” can include every fourteen days f two days, i.e., every twelve days to every fourteen days. “Once about every three weeks” can include every twenty-one days f three days, i.e., every eighteen days to every twenty-four days. Similar approximations apply, for example, to once about every four weeks, once about every five weeks, once about every six weeks, and once about every twelve weeks. In some embodiments, a dosing interval of once about every six weeks or once about every twelve weeks means that the first dose can be administered any day in the first week, and then the next dose can be administered any day in the sixth or twelfth week, respectively. In other embodiments, a dosing interval of once about every six weeks or once about every twelve weeks means that the first dose is administered on a particular day of the first week (e.g., Monday) and then the next dose is administered on the same day of the sixth or twelfth weeks (i.e., Monday), respectively.

As described herein, any concentration range, percentage range, ratio range, or integer range is to be understood to include the value of any integer within the recited range and, when appropriate, fractions thereof (such as one tenth and one hundredth of an integer), unless otherwise indicated.

Various aspects of the disclosure are described in further detail in the following subsections.

### II. Anti-CD30 Antibodies and Antibody-Drug Conjugates

A. Ani-CD30 Antibody

In one aspect, the therapy of the present disclosure utilizes an anti-CD30 antibody or an antigen-binding fragment thereof. CD30 receptors are members of the tumor necrosis factor receptor superfamily involved in limiting the proliferative potential of autoreactive CD8 effector T cells. Antibodies targeting CD30 can potentially be either agonists or antagonists of these CD30 mediated activities. In some embodiments, the anti-CD30 antibody is conjugated to a therapeutic agent (e.g., an anti-CD30 antibody-drug conjugate).

Murine anti-CD30 mAbs known in the art have been generated by immunization of mice with Hodgkin's disease (HD) cell lines or purified CD30 antigen. AC10, originally termed C10 (Bowen et al., 1993, J. Immunol. 151:5896 5906), is distinct in that this anti-CD30 mAb that was prepared against a hum an NK-like cell line, YT (Bowen et al., 1993, J. Immunol. 151:5896 5906). Initially, the signaling activity of this mAb was evidenced by the down regulation of the cell surface expression of CD28 and CD45 molecules, the up regulation of cell surface CD25 expression and the induction of homotypic adhesion following binding of C10 to YT cells. Sequences of the AC10 antibody are set out in SEQ ID NO: 1-16. See also U.S. Pat. No. 7,090,843, incorporated herein by reference.

Generally, anti-CD30 antibodies of the disclosure bind CD30, e.g., human CD30, and exert cytostatic and cytotoxic effects on cells expressing CD30. Anti-CD30 antibodies of the disclosure are preferably monoclonal, and may be multispecific, human, humanized or chimeric antibodies, single chain antibodies, Fab fragments, F(ab′) fragments, fragments produced by a Fab expression library, and CD30 binding fragments of any of the above. In some embodiments, the anti-CD30 antibodies of the disclosure specifically bind CD30. The immunoglobulin molecules of the disclosure can be of any type (e.g., IgG, IgE, IgM, IgD, IgA and IgY), class (e.g., IgG1, IgG2, IgG3, IgG4, IgA1 and IgA2) or subclass of immunoglobulin molecule.

In certain embodiments of the disclosure, the anti-CD30 antibodies are antigen-binding fragments (e.g., human antigen-binding fragments) as described herein and include, but are not limited to, Fab, Fab′ and F(ab′)₂, Fd, single-chain Fvs (scFv), single-chain antibodies, disulfide-linked Fvs (sdFv) and fragments comprising either a V_{L} or V_{H} domain. Antigen-binding fragments, including single-chain antibodies, may comprise the variable region(s) alone or in combination with the entirety or a portion of the following: hinge region, CH1, CH2, CH3 and CL domains. Also included in the present disclosure are antigen-binding fragments comprising any combination of variable region(s) with a hinge region, CH1, CH2, CH3 and CL domains. In some embodiments, the anti-CD30 antibodies or antigen-binding fragments thereof are human, murine (e.g., mouse and rat), donkey, sheep, rabbit, goat, guinea pig, camelid, horse, or chicken.

The anti-CD30 antibodies of the present disclosure may be monospecific, bispecific, trispecific or of greater multi specificity. Multispecific antibodies may be specific for different epitopes of CD30 or may be specific for both CD30 as well as for a heterologous protein. See, e.g., PCT publications WO 93/17715; WO 92/08802: WO 91/00360; WO 92/05793; Tutt, et al., 1991, J. Immunol. 147:60 69; U.S. Pat. Nos. 4,474,893; 4,714,681; 4,925,648; 5,573,920; 5,601,819; Kostelny et al., 1992, J. Immunol. 148:1547 1553.

Anti-CD30 antibodies of the present disclosure may be described or specified in terms of the particular CDRs they comprise. In certain embodiments antibodies of the disclosure comprise one or more CDRs of AC10. The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, Md. (“Kabat” numbering scheme); Al-Lazikani et al., (1997) JMB 273, 927-948 (“Chothia” numbering scheme); MacCallum et al., J. Mol. Biol. 262:732-745 (1996), “Antibody-antigen interactions: Contact analysis and binding site topography,” J. Mol. Biol. 262, 732-745.” (“Contact” numbering scheme); Lefranc M P et al., “IMGT unique numbering for immunoglobulin and T cell receptor variable domains and Ig superfamily V-like domains,” Dev Comp Immunol, 2003 January; 27(1):55-77 (“IMGT” numbering scheme); Honegger A and Pluckthun A, “Yet another numbering scheme for immunoglobulin variable domains: an automatic modeling and analysis tool,” J Mol Biol, 2001 Jun. 8; 309(3):657-70, (“Aho” numbering scheme); and Martin et al., “Modeling antibody hypervariable loops: a combined algorithm,” PNAS, 1989, 86(23):9268-9272, (“AbM” numbering scheme). The boundaries of a given CDR may vary depending on the scheme used for identification. In some embodiments, a “CDR” or “complementarity determining region,” or individual specified CDRs (e.g., CDR-H1, CDR-H2, CDR-H3), of a given antibody or region thereof (e.g., variable region thereof) should be understood to encompass a (or the specific) CDR as defined by any of the aforementioned schemes. For example, where it is stated that a particular CDR (e.g., a CDR-H3) contains the amino acid sequence of a corresponding CDR in a given V_{H} or V_{L} region amino acid sequence, it is understood that such a CDR has a sequence of the corresponding CDR (e.g., CDR-H3) within the variable region, as defined by any of the aforementioned schemes. The scheme for identification of a particular CDR or CDRs may be specified, such as the CDR as defined by the Kabat, Chothia, AbM or IMGT method.

The disclosure encompasses an antibody or derivative thereof comprising a heavy or light chain variable domain, said variable domain comprising (a) a set of three CDRs, in which said set of CDRs are from monoclonal antibody AC10, and (b) a set of four framework regions, in which said set of framework regions differs from the set of framework regions in monoclonal antibody AC10, and in which said antibody or derivative thereof immunospecifically binds CD30.

In one aspect, the anti-CD30 antibody is AC10. In some embodiments, the anti-CD30 antibody is cAC10. cAC10 is a chimeric IgG1 monoclonal antibody that specifically binds CD30. cAC10 induces growth arrest of CD30⁺ cell lines in vtro and has pronounced antitumor activity in severe combined immunodeficiency (SCID) mouse xenograft models of Hodgkin disease. See Francisco et al., Blood 102(4):1458-64 (2003). AC10 antibody and cAC10 antibody are described in U.S. Pat. Nos. 9,211,319 and 7,090,843. cAC10 is also known as brentuximab.

In one aspect, anti-CD30 antibodies that compete with AC10 antibody and/or cAC10 antibody binding to CD30 are provided. Anti-CD30 antibodies that bind to the same epitope as AC10 antibody and cAC10 antibody are also provided.

In one aspect, provided herein is an anti-CD30 antibody comprising 1, 2, 3, 4, 5, or 6 of the CDR sequences of the AC10 antibody. In one aspect, provided herein is an anti-CD30 antibody comprising 1, 2, 3, 4, 5, or 6 of the CDR sequences of the cAC10 antibody. In some embodiments, the CDR is a Kabat CDR or a Chothia CDR.

In one aspect, provided herein is an anti-CD30 antibody comprising a heavy chain variable region and a light chain variable region, wherein the heavy chain variable region comprises (i) CDR-H1 comprising the amino acid sequence of SEQ ID NO:1, (ii) CDR-H2 comprising the amino acid sequence of SEQ ID NO:2, and (iii) CDR-H3 comprising the amino acid sequence of SEQ ID NO:3; and/or wherein the light chain variable region comprises (i) CDR-L1 comprising the amino acid sequence of SEQ ID NO:4, (ii) CDR-L2 comprising the amino acid sequence of SEQ ID NO:5, and (iii) CDR-L3 comprising the amino acid sequence of SEQ ID NO:6.

An anti-CD30 antibody described herein may comprise any suitable framework variable domain sequence, provided that the antibody retains the ability to bind CD30 (e.g., human CD30). As used herein, heavy chain framework regions are designated “HC-FR1-FR4,” and light chain framework regions are designated “LC-FR1-FR4.” In some embodiments, the anti-CD30 antibody comprises a heavy chain variable domain framework sequence of SEQ ID NO:9, 10, 11, and 12 (HC-FR1, HC-FR2, HC-FR3, and HC-FR4, respectively). In some embodiments, the anti-CD30 antibody comprises a light chain variable domain framework sequence of SEQ ID NO:13, 14, 15, and 16 (LC-FR1, LC-FR2, LC-FR3, and LC-FR4, respectively).

In one embodiment, an anti-CD30 antibody comprises a heavy chain variable domain comprising a framework sequence and hypervariable regions, wherein the framework sequence comprises the HC-FR1-HC-FR4 amino acid sequences of SEQ ID NO:9 (HC-FR1), SEQ ID NO:10 (HC-FR2), SEQ ID NO: 11 (HC-FR3), and SEQ ID NO: 12 (HC-FR4), respectively; the CDR-H1 comprises the amino acid sequence of SEQ ID NO:1; the CDR-H2 comprises the amino acid sequence of SEQ ID NO:2; and the CDR-H3 comprises the amino acid sequence of SEQ ID NO:3.

In one embodiment, an anti-CD30 antibody comprises a light chain variable domain comprising a framework sequence and hypervariable regions, wherein the framework sequence comprises the LC-FR1-LC-FR4 amino acid sequences of SEQ ID NO: 13 (LC-FR1), SEQ ID NO:14 (LC-FR2), SEQ ID NO:15 (LC-FR3), and SEQ ID NO:16 (LC-FR4), respectively; the CDR-L1 comprises the amino acid sequence of SEQ ID NO:4; the CDR-L2 comprises the amino acid sequence of SEQ ID NO:5; and the CDR-L3 comprises the amino acid sequence of SEQ ID NO:6.

In some embodiments of the anti-CD30 antibodies described herein, the heavy chain variable domain comprises the amino acid sequence of QIQLQQSGPEVVKPGASVKISCKASGYTFTDYYITWVKQKPGQGLEWIGWIYPGSGNTK YNEKFKGKATLTVDTSSSTAFMQLSSLTSEDTAVYFCANYGNYWFAYWGQGTQVTVS A (SEQ ID NO:7) and the light chain variable domain comprises the amino acid sequence of DIVLTQSPASLAVSLGQRATISCKASQSVDFDGDSYMNWYQQKPGQPPKVLIYAASNLE SGIPARFSGSGSGTDFTLNIHPVEEEDAATYYCQQSNEDPWTFGGGTKLEIK (SEQ ID NO:8).

In some embodiments of the anti-CD30 antibodies described herein, the heavy chain CDR sequences comprise the following:

a) CDR-H1
(DYYIT (SEQ ID NO: 1));
b) CDR-H2
(WIYPGSGNTKYNEKFKG (SEQ ID NO: 2));
and
c) CDR-H3
(YGNYWFAY (SEQ ID NO: 3)).

In some embodiments of the anti-CD30 antibodies described herein, the heavy chain FR sequences comprise the following:

a) HC-FR1
(QIQLQQSGPEVVKPGASVKISCKASGYTFT (SEQ ID NO: 9));
b) HC-FR2
(WVKQKPGQGIEWIG (SEQ ID NO: 10));
c) HC-FR3
(KATLTVDTSSSTAFMQLSSLTSEDTAVYFCAN (SEQ ID NO: 11));
and
d) HC-FR4
(WGQGTQVTVSA (SEQ ID NO: 12)).

In some embodiments of the anti-CD30 antibodies described herein, the light chain CDR sequences comprise the following:

a) CDR-L1
(KASQSVDFDGDSYMN (SEQ ID NO: 4));
b) CDR-L2
(AASNLES (SEQ ID NO: 5));
and
c) CDR-L3
(QQSNEDPWT (SEQ ID NO: 6)).

In some embodiments of the anti-CD30 antibodies described herein, the light chain FR sequences comprise the following:

a) LC-FR1
(DIVLTQSPASLAVSLGQRATISC (SEQ ID NO: 13));
b) LC-FR2
(WYQQKPGQPPKVLIY (SEQ ID NO: 14));
c) LC-FR3
(GIPARFSGSGSGTDFTLNIHPVEEEDAATYYC (SEQ ID NO: 15));
and
d) LC-FR4
(FGGGTKLEIK (SEQ ID NO: 16)).

In some embodiments, provided herein is an anti-CD30 antibody that binds to CD30 (e.g., human CD30), wherein the antibody comprises a heavy chain variable region and a light chain variable region, wherein the antibody comprises:
(a) heavy chain variable domain comprising:
(1) an HC-FR1 comprising the amino acid sequence of SEQ ID NO:9;
(2) an CDR-H1 comprising the amino acid sequence of SEQ ID NO:1;
(3) an HC-FR2 comprising the amino acid sequence of SEQ ID NO:10;
(4) an CDR-H2 comprising the amino acid sequence of SEQ ID NO:2;
(5) an HC-FR3 comprising the amino acid sequence of SEQ ID NO:11;
(6) an CDR-H3 comprising the amino acid sequence of SEQ ID NO:3; and
(7) an HC-FR4 comprising the amino acid sequence of SEQ ID NO:12, and/or
(b) a light chain variable domain comprising:
(1) an LC-FR1 comprising the amino acid sequence of SEQ ID NO: 13
(2) an CDR-L1 comprising the amino acid sequence of SEQ ID NO:4;
(3) an LC-FR2 comprising the amino acid sequence of SEQ ID NO: 14;
(4) an CDR-L2 comprising the amino acid sequence of SEQ ID NO:5;
(5) an LC-FR3 comprising the amino acid sequence of SEQ ID NO 15;
(6) an CDR-L3 comprising the amino acid sequence of SEQ ID NO:6: and
(7) an LC-FR4 comprising the amino acid sequence of SEQ ID NO: 16.

In one aspect, provided herein is an anti-CD30 antibody comprising a heavy chain variable domain comprising the amino acid sequence of SEQ ID NO:7 and/or comprising a light chain variable domain comprising the amino acid sequence of SEQ ID NO:8.

In some embodiments, provided herein is an anti-CD30 antibody comprising a heavy chain variable domain comprising an amino acid sequence having at least 85%, 86%, 87%, 88%, 89%, 90%, 91%, 92%, 93%, 94%, 95%, 96%, 97%, 98%, or 99% sequence identity to the amino acid sequence of SEQ ID NO:7. In certain embodiments, a heavy chain variable domain comprising an amino acid sequence having at least 85%, 86%, 87%, 88%, 89%, 90°, 91%, 92%, 93%, 94%, 95%, 96%, 97%, 98%, or 99% sequence identity to the amino acid sequence of SEQ ID NO:7 contains substitutions (e.g., conservative substitutions), insertions, or deletions relative to the reference sequence and retains the ability to bind to a CD30 (e.g., human CD30). In certain embodiments, a total of 1 to 10 amino acids have been substituted, inserted and/or deleted in SEQ ID NO:7. In certain embodiments, substitutions, insertions, or deletions (e.g., 1, 2, 3, 4, or 5 amino acids) occur in regions outside the CDR s (i.e., in the FRs). In some embodiments, the anti-CD30 antibody comprises a heavy chain variable domain sequence of SEQ ID NO:7 including post-translational modifications of that sequence. In a particular embodiment, the heavy chain variable domain comprises one, two or three CDRs selected from: (a) CDR-H1 comprising the amino acid sequence of SEQ ID NO:1, (b) CDR-H2 comprising the amino acid sequence of SEQ ID NO:2, and (c) CDR-H3 comprising the amino acid sequence of SEQ ID NO:3.

In some embodiments, provided herein is an anti-CD30 antibody comprising a light chain variable domain comprising an amino acid sequence having at least 85%, 86%, 87%, 88%, 89%, 90%, 91%, 92%, 93%, 94%, 95%, 96%, 97%, 98%, or 99% sequence identity to the amino acid sequence of SEQ ID NO:8. In certain embodiments, a light chain variable domain comprising an amino acid sequence having at least 85%, 86%, 87%, 88%, 89%, 90%, 91%, 92%, 93%, 94%, 95%, 96%, 97%, 98%, or 99% sequence identity to the amino acid sequence of SEQ ID NO:8 contains substitutions (e.g., conservative substitutions), insertions, or deletions relative to the reference sequence and retains the ability to bind to a CD30 (e.g., human CD30). In certain embodiments, a total of 1 to 10 amino acids have been substituted, inserted and/or deleted in SEQ ID NO:8. In certain embodiments, substitutions, insertions, or deletions (e.g., 1, 2, 3, 4, or 5 amino acids) occur in regions outside the CDR s (i.e., in the FRs). In some embodiments, the anti-CD30 antibody comprises a light chain variable domain sequence of SEQ ID NO:8 including post-translational modifications of that sequence. In a particular embodiment, the light chain variable domain comprises one, two or three CDRs selected from: (a) CDR-H1 comprising the amino acid sequence of SEQ ID NO:4, (b) CDR-H2 comprising the amino acid sequence of SEQ ID NO:5, and (c) CDR-H3 comprising the amino acid sequence of SEQ ID NO:6.

In some embodiments, the anti-CD30 antibody comprises a heavy chain variable domain as in any of the embodiments provided above, and a light chain variable domain as in any of the embodiments provided above. In one embodiment, the antibody comprises the heavy chain variable domain sequence of SEQ ID NO:7 and the light chain variable domain sequence of SEQ ID NO:8, including post-translational modifications of those sequences.

In some embodiments, the anti-CD30 antibody of the anti-CD30 antibody-drug conjugate comprises: i) a heavy chain CDR1 set out in SEQ ID NO: 1, a heavy chain CDR2 set out in SEQ ID NO: 2, a heavy chain CDR3 set out in SEQ ID NO: 3; and ii) a light chain CDR1 set out in SEQ ID NO: 4, a light chain CDR2 set out in SEQ ID NO: 5, and a light chain CDR3 set out in SEQ ID NO: 6.

In some embodiments, the anti-CD30 antibody of the anti-CD30 antibody-drug conjugate comprises: i) an amino acid sequence at least 85% identical to a heavy chain variable region set out in SEQ ID NO: 7, and ii) an amino acid sequence at least 85% identical to a light chain variable region set out in SEQ ID NO: 8.

In some embodiments, the anti-CD30 antibody of the anti-CD30 antibody-drug conjugate is a monoclonal antibody.

In some embodiments, the anti-CD30 antibody of the anti-CD30 antibody-drug conjugate is a chimeric AC10 antibody. In some embodiments, the anti-CD30 antibody of the anti-CD30 antibody-drug conjugate is brentuximab.

Antibodies of the present invention may also be described or specified in terms of their binding affinity to CD30. Preferred binding affinities include those with a dissociation constant or Kd less than 5×10⁻² M, 10⁻² M, 5×10⁻³ M, 10⁻³ M, 5×10⁻⁴ M, 10⁻⁴ M, 5×10⁻⁵ M, 10⁻⁵ M, 5×10⁻⁶M, 10⁻⁶ M, 5×10⁻⁷ M, 10⁻⁷ M, 5×10⁻⁸ M, 10⁻⁸M, 5×10⁻⁹M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹³ M, 5×10⁻¹⁴ M, 10⁻¹⁴ M, 5×10⁻¹⁵ M, or 10⁻¹⁵ M.

There are five classes of immunoglobulins: IgA, IgD, IgE, IgG and IgM, having heavy chains designated α, δ, ε, γ and β, respectively. The γ and a classes are further divided into subclasses e.g., humans express the following subclasses: IgG1, IgG2, IgG3, IgG4, IgA1 and IgA2. IgG1 antibodies can exist in multiple polymorphic variants termed allotypes (reviewed in Jefferis and Lefranc 2009. mAbs Vol 1 Issue 4 1-7) any of which are suitable for use in some of the embodiments herein. Common allotypic variants in human populations are those designated by the letters a, f, n, z or combinations thereof. In any of the embodiments herein, the antibody may comprise a heavy chain Fc region comprising a human IgG Fc region. In further embodiments, the human IgG Fc region comprises a human IgG1.

In one aspect of the invention, polynucleotides encoding anti-CD30 antibodies, such as those anti-CD30 antibodies described herein, are provided. In certain embodiments, vectors comprising polynucleotides encoding anti-CD30 antibodies as described herein are provided. In certain embodiments, host cells comprising such vectors are provided. In another aspect of the invention, compositions comprising anti-CD30 antibodies described herein or polynucleotides encoding anti-CD30 antibodies described herein are provided.

The antibodies also include derivatives that are modified, i.e., by the covalent attachment of any type of molecule to the antibody such that covalent attachment does not prevent the antibody from binding to CD30 or from exerting a cytostatic or cytotoxic effect on HD cells. For example, but not by way of limitation, the antibody derivatives include antibodies that have been modified, e.g., by glycosylation, acetylation, PEGylation, phosphylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

B. Antibody-Drug Conjugate Structure

In some embodiments, the anti-CD30 antibody is conjugated to a therapeutic agent (e.g., an anti-CD30 antibody-drug conjugate). In some embodiments, the therapeutic agent comprises an anti-neoplastic agent (e.g., an anti-mitotic agent). In certain embodiments, the therapeutic agent is an auristatin. In certain embodiments, the therapeutic agent is selected from the group consisting of monomethyl auristatin E (MMAE), monomethyl auristatin F (MMAF), auristatin drug analogues, cantansinoids, maytansinoids (e.g., maytansine; DMs), dolastatins, cryptophycin, duocarmycin, duocarmycin derivatives, esperamicin, calicheamicin, pyrolobenodiazepine (PBD), and any combination thereof. In one particular embodiment, the anti-CD30 antibody is conjugated to MMAE. The antibody can be conjugated to at least one, at least two, at least three, at least four, at least five, at least six, at least seven, at least eight, at least nine, or at least ten molecules of the therapeutic agent (e.g., MMAE). In one embodiment, the anti-CD30 antibody is conjugated to four molecules of the therapeutic agent, e.g., four molecules of MMAE. In one particular embodiment, the anti-CD30 antibody is conjugated to MMAF. The antibody can be conjugated to at least one, at least two, at least three, at least four, at least five, at least six, at least seven, at least eight, at least nine, or at least ten molecules of the therapeutic agent (e.g., MMAF). In one embodiment, the anti-CD30 antibody is conjugated to four molecules of the therapeutic agent, e.g., four molecules of MMAF.

In one embodiment, the auristatin is monomethyl auristatin E (MMAE):

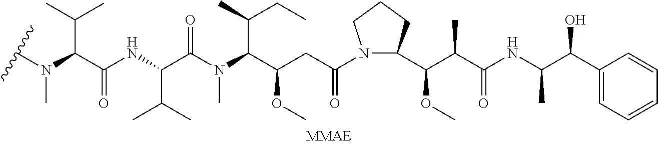 [SMILES: *N(C)[C@H](C(=O)N[C@H](C(=O)N(C)[C@@H]([C@@H](C)CC)[C@@H](CC(=O)N1CCC[C@H]1[C@H](OC)[C@@H](C)C(=O)N[C@H](C)[C@@H](O)c1ccccc1)OC)C(C)C)C(C)C]

wherein the wavy line indicates the attachment site for the linker.

In one embodiment, the auristatin is monomethyl auristatin F (MMAF):

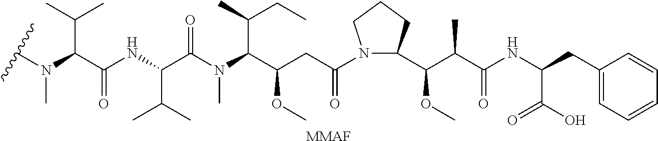

wherein the wavy line indicates the attachment site for the linker.

In some embodiments, the anti-CD30 antibody-drug conjugate further comprises a linker between the therapeutic agent and the antibody. In some embodiments, the linker comprises one or more naturally occurring amino acids, one or more non-naturally occurring (e.g., synthetic) amino acids, a chemical linker, or any combination thereof. In certain embodiments, the linker is a cleavable linker, e.g., a protease cleavable linker. In certain embodiments, the linker is specifically cleaved upon uptake by a target cell, e.g., upon uptake by a cell expressing CD30. In certain embodiments, the linker is a cleavable peptide linker having the formula: “-MC-vc-PAB-” or “-MC-val-cit-PAB-”, wherein “MC” refers to the stretcher maleimidocaproyl having the following structure:

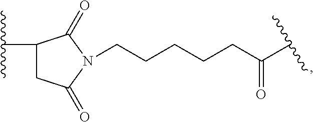 [SMILES: *C(=O)CCCCCN1C(=O)CC(*)C1=O]

“vc” and “val-cit” refer to the dipeptide valine-citrulline, and PAB refers to a self-immolative spacer having the following structure:

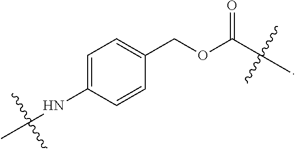 [SMILES: *Nc1ccc(COC(*)=O)cc1]

In some embodiments, cleavage of the linker activates a cytotoxic activity of the therapeutic agent. In certain embodiments, the linker is a non-cleavable linker. In certain embodiments, the non-cleavable linker has the formula: “-MC-”, wherein “MC” refers to the stretcher maleimidocaproyl having the following structure:

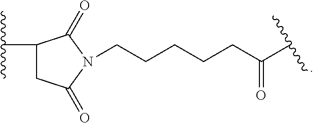 [SMILES: *C(=O)CCCCCN1C(=O)CC(*)C1=O]

In some embodiments, the antibody-drug conjugates comprises an anti-CD30 antibody, covalently linked to MMAE through a vc-PAB linker. In some embodiments, the antibody-drug conjugate is delivered to the subject as a pharmaceutical composition. In some embodiments, the CD30 antibody-drug conjugates contemplated herein are as described in U.S. Pat. No. 9,211,319, herein incorporated by reference.

In one embodiment, the anti-CD30 antibody-drug conjugate comprises brentuximab vedotin. In one particular embodiment, the anti-CD30 antibody-drug conjugate is brentuximab vedotin. Brentuximab vedotin (BV; also known as “ADCETRIS®”) is a CD30-directed antibody-drug conjugate (ADC) comprising a chimeric anti-CD30 antibody (cAC10), a therapeutic agent (MMAE), and a protease-cleavable linker between the cAC10 and the MMAE, as shown in the following structure:

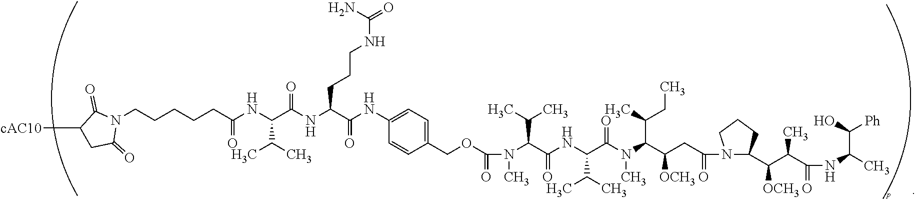

The drug to antibody ratio or drug loading is represented by “p” in the structure of brentuximab vedotin and ranges in integer values from 1 to 8. The average drug loading of brentuximab vedotin in a pharmaceutical composition is about 4. ADCETRIS® is approved by the FDA for treatment of patients with Hodgkin lymphoma after failure of autologous stem cell transplant (ASCT) or after failure of at least two prior multi-agent chemotherapy regimens in patients who are not ASCT candidates and for the treatment of patients with systemic anaplastic large cell lymphoma after failure of at least one prior multi-agent chemotherapy regimen.

In one embodiment, the anti-CD30 antibody is an anti-CD30 antibody or antigen-binding fragment thereof that binds to the same epitope as cAC10, e.g., the same epitope as brentuximab vedotin. In certain embodiments, the anti-CD30 antibody is an antibody that has the same CDRs as cAC10, e.g., the same CDRs as brentuximab vedotin. Antibodies that bind to the same epitope are expected to have functional properties very similar to those of cAC10 by virtue of their binding to the same epitope region of CD30. These antibodies can be readily identified based on their ability to, for example, cross-compete with cAC10 in standard CD30 binding assays such as Biacore analysis, ELISA assays, or flow cytometry.

In certain embodiments, the antibodies that cross-compete for binding to human CD30 with, or bind to the same epitope region of human CD30 as cAC10 are monoclonal antibodies. For administration to human subjects, these cross-competing antibodies can be chimeric antibodies, or can be humanized or human antibodies. Such chimeric, humanized, or human monoclonal antibodies can be prepared and isolated by methods well known in the art. Anti-CD30 antibodies usable in the methods of the disclosed disclosure also include antigen-binding portions of the above antibodies.

In other embodiments, the anti-CD30 antibody or antigen-binding portion thereof is a chimeric, humanized, or human monoclonal antibody or a portion thereof. In certain embodiments for treating a human subject, the antibody is a humanized antibody. In other embodiments for treating a human subject, the antibody is a human antibody. Antibodies of an IgG1, IgG2, IgG3, or IgG4 isotype can be used.

In one embodiment, the antibody-drug conjugate is brentuximab vedotin.

C. Nucleic Acids, Host Cells and Methods of Production

In some aspects, also provided herein are nucleic acids encoding an anti-CD30 antibody or antigen-binding fragment thereof as described herein. Further provided herein are vectors comprising the nucleic acids encoding an anti-CD30 antibody or antigen-binding fragment thereof as described herein. Further provided herein are host cells expressing the nucleic acids encoding an anti-CD30 antibody or antigen-binding fragment thereof as described herein. Further provided herein are host cells comprising the vectors comprising the nucleic acids encoding an anti-CD30 antibody or antigen-binding fragment thereof as described herein. Methods of producing an anti-CD30 antibody, linker and anti-CD30 antibody-drug conjugate are described in U.S. Pat. Nos. 7,090,843 and 9,211,319.

The anti-CD30 antibodies described herein may be prepared by well-known recombinant techniques using well known expression vector systems and host cells. In one embodiment, the antibodies are prepared in a CHO cell using the GS expression vector system as disclosed in De la Cruz Edmunds et al., 2006, Molecular Biotechnology 34; 179-190, EP216846, U.S. Pat. No. 5,981,216, WO 87/04462, EP323997, U.S. Pat. Nos. 5,591,639, 5,658,759, EP338841, U.S. Pat. Nos. 5,879,936, and 5,891,693.

After isolating and purifying the anti-CD30 antibodies from the cell media using well known techniques in the art, they are conjugated with an auristatin via a linker as described in U.S. Pat. No. 9,211,319.

Monoclonal anti-CD30 antibodies described herein may e.g. be produced by the hybridoma method first described by Kohler et al., Nature, 256, 495 (1975), or may be produced by recombinant DNA methods. Monoclonal antibodies may also be isolated from phage antibody libraries using the techniques described in, for example, Clackson et al., Nature, 352, 624-628 (1991) and Marks et al., J Mol, Biol., 222(3):581-597 (1991). Monoclonal antibodies may be obtained from any suitable source. Thus, for example, monoclonal antibodies may be obtained from hybridomas prepared from murine splenic B cells obtained from mice immunized with an antigen of interest, for instance in form of cells expressing the antigen on the surface, or a nucleic acid encoding an antigen of interest. Monoclonal antibodies may also be obtained from hybridomas derived from antibody-expressing cells of immunized humans or non-human mammals such as rats, dogs, primates, etc.

In one embodiment, the antibody (e.g., anti-CD30 antibody) of the invention is a human antibody. Human monoclonal antibodies directed against CD30 may be generated using transgenic or transchromosomal mice carrying parts of the human immune system rather than the mouse system. Such transgenic and transchromosomic mice include mice referred to herein as HuMAb mice and KM mice, respectively, and are collectively referred to herein as “transgenic mice”.

The HuMAb mouse contains a human immunoglobulin gene minilocus that encodes unrearranged human heavy (μ and μ) and a light chain immunoglobulin sequences, together with targeted mutations that inactivate the endogenous μ and κ chain loci (Lonberg, N. et al., Nature, 368, 856-859 (1994)). Accordingly, the mice exhibit reduced expression of mouse IgM or κ and in response to immunization, the introduced human heavy and light chain transgenes undergo class switching and somatic mutation to generate high affinity human IgG,κ monoclonal antibodies (Lonberg, N. et al. (1994), supra; reviewed in Lonberg, N. Handbook ofFxperimental Pharmacology 113, 49-101 (1994), Lonberg, N. and Huszar. D., Intern. Rev. Immunol, Vol. 13 65-93 (1995) and Harding, F. and Lonberg, N. Ann, N.Y. Acad. Sci 764:536-546 (1995)). The preparation of HuMAb mice is described in detail in Taylor, L. et al., Nucleic Acids Research. 20:6287-6295 (1992), Chen, J. et al., International Immunology. 5:647-656 (1993), Tuaillon at al., J. Immunol, 152:2912-2920 (1994), Taylor, L. et al., International Immunolog, 6:579-591 (1994), Fishwild, D. et al., Nature Biotechnology, 14:845-851 (1996). See also U.S. Pat. Nos. 5,545,806, 5,569,825, 5,625,126, 5,633,425, 5,789,650, 5,877,397, 5,661,016, 5,814,318, 5,874,299, 5,770,429, 5,545,807, WO 98/24884, WO 94/25585, WO 93/1227, WO 92/22645, WO 92/03918 and WO 01/09187.

The HCo7 mice have a JKD disruption in their endogenous light chain (kappa) genes (as described in Chen et al, EMBO J. 12:821-830 (1993)), a CMD disruption in their endogenous heavy chain genes (as described in Example 1 of WO 01/14424), a KCo5 human kappa light chain transgene (as described in Fishwild et al., Nature Biotechnology, 14:845-851 (1996)), and a HCo7 human heavy chain transgene (as described in U.S. Pat. No. 5,770,429).

The HCo12 mice have a JKD disruption in their endogenous light chain (kappa) genes (as described in Chen et al., EMBO J. 12:821-830 (1993)), a CMD disruption in their endogenous heavy chain genes (as described in Example 1 of WO 01/14424), a KCo5 human kappa light chain transgene (as described in Fishwild et al., Nature Biotechnology, 14:845-851 (1996)), and a HCol2 human heavy chain transgene (as described in Example 2 of WO 01/14424).

The HCol7 transgenic mouse strain (see also US 2010/0077497) was generated by coinjection of the 80 kb insert of pHC2 (Taylor et al. (1994) Int. Immunol., 6:579-591), the Kb insert of pVX6, and a −460 kb yeast artificial chromosome fragment of the yIgH24 chromosome. This line was designated (HCol7) 25950. The (HCo17) 25950 line was then bred with mice comprising the CMD mutation (described in Example 1 of PCT Publication WO 01109187), the JKD mutation (Chen et al, (1993) EMBO J 12:811-820), and the (KC05) 9272 transgene (Fishwild et al. (1996) Nature Biotechnology, 14:845-851). The resulting mice express human immunoglobulin heavy and kappa light chain trans genes in a background homozygous for disruption of the endogenous mouse heavy and kappa light chain loci.

The HCo20 transgenic mouse strain is the result of a co-injection of minilocus 30 heavy chain transgene pHC2, the germline variable region (Vh)-containing YAC ylgH10, and the minilocus construct pVx6 (described in WO09097006). The (HCo20) line was then bred with mice comprising the CMD mutation (described in Example 1 of PCT Publication WO 01/09187), the JKD mutation (Chen et al. (1993) EMBO J. 12:811-820), and the (KC05) 9272 trans gene (Fishwild eta). (1996) Nature Biotechnology, 14:845-851). The resulting mice express human 10 immunoglobulin heavy and kappa light chain transgenes in a background homozygous for disruption of the endogenous mouse heavy and kappa light chain loci.

In order to generate HuMab mice with the salutary effects of the Balb/c strain, HuMab mice were crossed with KC005 [MIK] (Balb) mice which were generated by backcrossing the KC05 strain (as described in Fishwild et (1996) Nature Biotechnology, 14:845-851) to wild-type Balb/c mice to generate mice as described in WO09097006. Using this crossing Balb/c hybrids were created for HCo12, HCo17, and HCo20 strains.

In the KM mouse strain, the endogenous mouse kappa light chain gene has been homozygously disrupted as described in Chen et al., EMBO J. 12:811-820 (1993) and the endogenous mouse heavy chain gene has been homozygously disrupted as described in Example 1 of WO 01/09187, This mouse strain carries a human kappa light chain transgene, KCo5, as described in Fishwild et al., Nature Biotechnology, 14:845-851 (1996). This mouse strain also carries a human heavy chain transchromosome composed of chromosome 14 fragment hCF (SC20) as described in WO 02/43478.

Splenocytes from these transgenic mice may be used to generate hybridomas that secrete human monoclonal antibodies according to well-known techniques, Human monoclonal or polyclonal antibodies of the present invention, or antibodies of the present invention originating from other species may also be generated transgenically through the generation of another non-human mammal or plant that is transgenic for the immunoglobulin heavy and light chain sequences of interest and production of the antibody in a recoverable form therefrom. In connection with the transgenic production in mammals, antibodies may be produced in, and recovered from, the milk of goats, cows, or other mammals. See for instance U.S. Pat. Nos. 5,827,690, 5,756,687, 5,750,172 and 5,741,957.

Further, human antibodies of the present invention or antibodies of the present invention from other species may be generated through display-type technologies, including, without limitation, phage display, retroviral display, ribosomal display, and other techniques, using techniques well known in the art and the resulting molecules may be subjected to additional maturation, such as affinity maturation, as such techniques are well known in the art (See for instance Hoogenboom et al., J. Mol, Biol. 227(2):381-388 (1992) (phage display), Vaughan et al., Nature Biotech, 14:309 (1996) (phage display), Hanes and Plucthau, PNAS USA 94:4937-4942 (1997) (ribosomal display), Parmley and Smith, Gene, 73:305-318 (1988) (phage display), Scott, TIBS. 17:241-245 (1992), Cwirla et al., PNAS USA, 87:6378-6382 (1990), Russel et al., Nucl. Acids Research, 21:10814085 (1993), Hogenboom et al., Immunol, Reviews, 130:43-68 (1992), Chiswell and McCafferty, TIBTECH, 10:80-84 (1992), and U.S. Pat. No. 5,733,743). If display technologies are utilized to produce antibodies that are not human, such antibodies may be humanized.

### III. Binding Assays and Other Assays

In one aspect, an antibody of the invention is tested for its antigen binding activity, for example, by known methods such as Enzyme-Linked Immunosorbant Assay (ELISA), immunoblotting (e.g., Western blotting), flow cytometry (e.g., FACS®), immunohistochemistry, immunofluorescence, etc.

In another aspect, competition assays may be used to identify an antibody that competes with any one of the antibodies described herein for binding to CD30 (e.g., brentuximab). Cross-competing antibodies can be readily identified based on their ability to cross-compete in standard CD30 binding assays such as Biacore analysis, ELISA assays or flow cytometry (See, e.g., WO 2013/173223). In certain embodiments, such a competing antibody binds to the same epitope (e.g., a linear or a conformational epitope) that is bound by any one of the antibodies disclosed herein (e.g., brentuximab). Detailed exemplary methods for mapping an epitope to which an antibody binds are provided in Morris “Epitope Mapping Protocols,” in Methods in Molecular Biology Vol. 66 (Humana Press, Totowa, N.J., 1996).

In an exemplary competition assay, immobilized CD30 is incubated in a solution comprising a first labeled antibody that binds to CD30 (e.g., brentuximab) and a second unlabeled antibody that is being tested for its ability to compete with the first antibody for binding to CD30. The second antibody may be present in a hybridoma supernatant. As a control, immobilized CD30 is incubated in a solution comprising the first labeled antibody but not the second unlabeled antibody. After incubation under conditions permissive for binding of the first antibody to CD30, excess unbound antibody is removed, and the amount of label associated with immobilized CD30 is measured. If the amount of label associated with immobilized CD30 is substantially reduced in the test sample relative to the control sample, then that indicates that the second antibody is competing with the first antibody for binding to CD30. See, e.g., Harlow et al. Antibodies: A Laboratory Manual. Ch. 14 (Cold Spring Harbor Laboratory, Cold Spring Harbor, N.Y., 1988). In some embodiments, an anti-CD30 antibody competes for binding to CD30 with another anti-CD30 antibody (e.g., brentuximab) if the antibody blocks binding of the other antibody to CD30 in a competition assay by more than 20%, more than 25%, more than 30%, more than 35%, more than 40%, more than 45%, more than 50%, more than 55%, more than 60%, more than 65%, more than 70%, more than 75%, more than 80%, more than 85%, more than 90%, or more than 95%. In some embodiments, an anti-CD30 antibody does not compete for binding to CD30 with another anti-CD30 antibody (e.g., brentuximab) if the antibody blocks binding of the other antibody to CD30 in a competition assay by less than 20%, less than 15%, less than 10%, less than 9%, less than 8%, less than 7%, less than 6%, less than 5%, less than 4%, less than 3%, less than 2% , or less than 1%. In some embodiments, the CD30 is human CD30.

### IV. Methods of Treatment

A. HIV Infection

Human immunodeficiency virus infection and acquired immune deficiency syndrome (HIV/AIDS) is a spectrum of conditions caused by infection with the human immunodeficiency virus (HIV) Two types of HIV have been characterized: HIV-1 and HIV-2 IV is a retrovirus that primarily infects components of the human immune system such as CD4⁺ T-cell lymphocytes, macrophages and dendritic cells. It directly and indirectly destroys CD4⁺ T cells. CD4⁺ T-cell lymphocytes play a major role of protecting the human body of viruses and fungi, thus in there destruction, the host becomes immunodeficient, making the infected patients susceptible to infection by additional viruses (which can lead to cancers, such as lymphoma) and fungi.

Even with treatment, HIV viral reservoirs persist in infected cells. T regulatory cells (Tregs) have been implicated as a possible reservoir for HIV. Tregs have been demonstrated to express CD30.

In 2016, worldwide about 36.7 million people were living with HIV and it resulted in 1 million deaths. From the time AIDS was identified in the early 1980s to 2017, the disease has caused an estimated 35 million deaths worldwide.

Despite highly active combined antiretroviral therapy (cART), viral reservoirs persist in infected cells in individuals on cART. There are few therapeutic strategies to decrease the number of these persistently infected cells, and novel approaches to eliminate or reduce HIV reservoir burden and to increase CD4⁺ T-cell lymphocyte counts are urgently needed.

The invention provides methods for treating an HIV infection in a subject with an anti-CD30 antibody-drug conjugate described herein. The invention also provides methods for increasing CD4⁺ T-cell lymphocyte count in a subject infected with HIV with an anti-CD30 antibody-drug conjugate described herein. In some embodiments, the anti-CD30 antibody-drug conjugate comprises the complementary determining regions (CDRs) of brentuximab vedotin, or a biosimilar thereof. In some embodiments, the anti-CD30 antibody-drug conjugate comprises the complementary determining regions (CDRs) of brentuximab vedotin. In some embodiments, the anti-CD30 antibody-drug conjugate comprises the heavy chain variable region and the light chain variable region of brentuximab vedotin, or a biosimilar thereof. In some embodiments, the anti-CD30 antibody-drug conjugate comprises the heavy chain variable region and the light chain variable region of brentuximab vedotin. In some embodiments, the anti-brentuximab antibody-drug conjugate comprises a heavy chain variable region and a light chain variable region, wherein the heavy chain variable region comprises:

(i) a CDR-H1 comprising the amino acid sequence of SEQ ID NO: 1;

(ii) a CDR-H2 comprising the amino acid sequence of SEQ ID NO: 2; and

(iii) a CDR-H3 comprising the amino acid sequence of SEQ ID NO: 3; and wherein the light chain variable region comprises:

(i) a CDR-L1 comprising the amino acid sequence of SEQ ID NO: 4;

(ii) a CDR-L2 comprising the amino acid sequence of SEQ ID NO: 5; and

(iii) a CDR-L3 comprising the amino acid sequence of SEQ ID NO: 6. In some embodiments, the antibody-drug conjugate is brentuximab vedotin. In a particular embodiment, the subject is a human.

In another aspect, the present invention provides an antibody-drug conjugate that binds to CD30 as described herein for treating an HIV infection in a subject. In another aspect, the present invention provides an antibody-drug conjugate that binds to CD30 as described herein for increasing CD4⁺ T-cell lymphocyte count in a subject infected with HIV. In some embodiments, the anti-CD30 antibody-drug conjugate comprises the complementary determining regions (CDRs) of brentuximab vedotin, or a biosimilar thereof. In some embodiments, the anti-CD30 antibody-drug conjugate comprises the complementary determining regions (CDRs) of brentuximab vedotin. In some embodiments, the anti-CD30 antibody-drug conjugate comprises the heavy chain variable region and the light chain variable region of brentuximab vedotin, or a biosimilar thereof. In some embodiments, the anti-CD30 antibody-drug conjugate comprises the heavy chain variable region and the light chain variable region of brentuximab vedotin. In some embodiments, the anti-brentuximab antibody-drug conjugate comprises a heavy chain variable region and a light chain variable region, wherein the heavy chain variable region comprises:

(i) a CDR-H1 comprising the amino acid sequence of SEQ ID NO: 1;

(ii) a CDR-H2 comprising the amino acid sequence of SEQ ID NO: 2; and

(iii) a CDR-H3 comprising the amino acid sequence of SEQ ID NO: 3; and wherein the light chain variable region comprises:

(i) a CDR-L1 comprising the amino acid sequence of SEQ ID NO: 4;

(ii) a CDR-L2 comprising the amino acid sequence of SEQ ID NO: 5; and

(iii) a CDR-L3 comprising the amino acid sequence of SEQ ID NO: 6. In some embodiments, the antibody-drug conjugate is brentuximab vedotin. In a particular embodiment, the subject is a human.

In some embodiments, the subject has an HIV infection that is an HIV-1 infection or an HIV-2 infection. In some embodiments, the subject has an HIV-1 infection. In some embodiments, the subject has an HIV-2 infection.

In some embodiments, the subject does not have a hematologic cancer at the time of administration of an anti-CD30 antibody-drug conjugate described herein. In some embodiments, the subject does not have a hematologic cancer at the time of receiving a first dose of an anti-CD30 antibody-drug conjugate described herein. In some embodiments, the subject has not had a hematologic cancer for at least 3 months prior to the administration of an anti-CD30 antibody-drug conjugate described herein. In some embodiments, the subject has not had a hematologic cancer for at least 6 months prior to the administration of an anti-CD30 antibody-drug conjugate described herein. In some embodiments, the subject has not had a hematologic cancer for at least 9 months prior to the administration of an anti-CD30 antibody-drug conjugate described herein. In some embodiments, the subject has not had a hematologic cancer for at least 12 months prior to the administration of an anti-CD30 antibody-drug conjugate described herein. In some embodiments, the subject has not had a hematologic cancer for at least 18 months prior to the administration of an anti-CD30 antibody-drug conjugate described herein. In some embodiments, the subject has not had a hematologic cancer for at least 24 months prior to the administration of an anti-CD30 antibody-drug conjugate described herein. In some embodiments, the subject has never had a hematologic cancer. In some embodiments, the hematologic cancer is selected from the group consisting of classical Hodgkin Lymphoma, non-Hodgkin Lymphoma, cutaneous T-cell lymphoma (CTCL), and anaplastic large cell lymphoma (ALCL). In some embodiments, the hematologic cancer is classical Hodgkin Lymphoma. In some embodiments, the classical Hodgkin Lymphoma is a stage IIA with bulky disease, stage IIB, stage III or stage IV classical Hodgkin Lymphoma. In some embodiments, the hematologic cancer is non-Hodgkin Lymphoma. In some embodiments, the hematologic cancer is cutaneous T-cell lymphoma (CTCL). In some embodiments, the hematologic cancer is anaplastic large cell lymphoma (ALCL). In some embodiments, the ALCL is a systemic anaplastic large cell lymphoma (sALCL). In some embodiments the ALCL a primary cutaneous anaplastic large cell lymphoma (pcALCL). In some embodiments, the cutaneous T-cell lymphoma (CTCL) is a mycosis fungoides (MF). In some embodiments, the mycosis fungoides (MF) is a CD30⁺ ME.

In some embodiments, the subject has not been administered an anti-CD30 antibody-drug conjugate described herein prior to the administration of an anti-CD30 antibody-drug conjugate described herein to treat HIV infection in the subject. In some embodiments, the subject has not been administered an anti-CD30 antibody-drug conjugate described herein prior to the administration of an anti-CD30 antibody-drug conjugate described herein to increase CD4⁺ T-cell lymphocyte count in the subject.

In some embodiments, the number of CD4⁺ T-cell lymphocytes in a sample from the subject is assessed prior to the administration, e.g., the first administration, of an anti-CD30 antibody-drug conjugate described herein. In some embodiments, the subject has a CD4 lymphocyte count of <400 cells/mm₃ prior to administration of an anti-CD30 antibody-drug conjugate described herein. In some embodiments, the subject has a CD4 lymphocyte count of <400 cells/mm³ prior to receiving a first dose of an anti-CD30 antibody-drug conjugate described herein. In some embodiments, the subject has a CD4 lymphocyte count of <350 cells/mm³ prior to administration of an anti-CD30 antibody-drug conjugate described herein. In some embodiments, the subject has a CD4 lymphocyte count of <350 cells/mm³ prior to receiving a first dose of an anti-CD30 antibody-drug conjugate described herein. In some embodiments, the subject has a CD4 lymphocyte count of <300 cells/mm³ prior to administration of an anti-CD30 antibody-drug conjugate described herein. In some embodiments, the subject has a CD4 lymphocyte count of <300 cells/mm³ prior to receiving a first dose of an anti-CD30 antibody-drug conjugate described herein. In some embodiments, the subject has a CD4 lymphocyte count of <250 cells/mm³ prior to administration of an anti-CD30 antibody-drug conjugate described herein. In some embodiments, the subject has a CD4 lymphocyte count of <250 cells/mm³ prior to receiving a first dose of an anti-CD30 antibody-drug conjugate described herein. In some embodiments, the subject has a CD4 lymphocyte count of <200 cells/mm³ prior to administration of an anti-CD30 antibody-drug conjugate described herein. In some embodiments, the subject has a CD4 lymphocyte count of <200 cells/mm³ prior to receiving a first dose of an anti-CD30 antibody-drug conjugate described herein. In some embodiments, the subject has a CD4 lymphocyte count of <150 cells/mm³ prior to administration of an anti-CD30 antibody-drug conjugate described herein. In some embodiments, the subject has a CD4 lymphocyte count of <150 cells/mm³ prior to receiving a first dose of an anti-CD30 antibody-drug conjugate described herein. In some embodiments, the subject has a CD4 lymphocyte count of <100 cells/mm³ prior to administration of an anti-CD30 antibody-drug conjugate described herein. In some embodiments, the subject has a CD4 lymphocyte count of <100 cells/mm³ prior to receiving a first dose of an anti-CD30 antibody-drug conjugate described herein. In some embodiments, the subject has a CD4 lymphocyte count of <50 cells/mm³ prior to administration of an anti-CD30 antibody-drug conjugate described herein. In some embodiments, the subject has a CD4 lymphocyte count of <50 cells/mm³ prior to receiving a first dose of an anti-CD30 antibody-drug conjugate described herein. In some embodiments, the subject has a CD4 lymphocyte count of >50 cells/mm³ prior to administration of an anti-CD30 antibody-drug conjugate described herein. In some embodiments, the subject has a CD4 lymphocyte count of >50 cells/mm³ prior to receiving a first dose of an anti-CD30 antibody-drug conjugate described herein. In some embodiments, the subject has a CD4 lymphocyte count of >50 cells/mm³ and <200 cells/mm³ prior to administration of an anti-CD30 antibody-drug conjugate described herein. In some embodiments, the subject has a CD4 lymphocyte count of >50 cells/mm³ and <200 cells/mm³ prior to receiving a first dose of an anti-CD30 antibody-drug conjugate described herein.

In some embodiments, the HIV viral load in a sample from the subject is assessed prior to the administration, e.g., the first administration, of an anti-CD30 antibody-drug conjugate described here. In some embodiments, the sample is a plasma sample. In some embodiments, the subject has plasma HIV RNA ≥10,000 copies/mL prior to administration of an anti-CD30 antibody-drug conjugate described herein. In some embodiments, the subject has plasma HIV RNA ≥10,000 copies/mL prior to receiving a first dose of an anti-CD30 antibody-drug conjugate described herein. In some embodiments, the subject has plasma HIV RNA ≥5000 copies/mL prior to administration of an anti-CD30 antibody-drug conjugate described herein. In some embodiments, the subject has plasma HIV RNA ≥5000 copies/mL prior to receiving a first dose of an anti-CD30 antibody-drug conjugate described herein. In some embodiments, the subject has plasma HIV RNA ≥2000 copies/mL prior to administration of an anti-CD30 antibody-drug conjugate described herein. In some embodiments, the subject has plasma HIV RNA ≥2000 copies/mL prior to receiving a first dose of an anti-CD30 antibody-drug conjugate described herein. In some embodiments, the subject has plasma HIV RNA ≥1000 copies/mL prior to administration of an anti-CD30 antibody-drug conjugate described herein. In some embodiments, the subject has plasma HIV RNA ≥1000 copies/mL prior to receiving a first dose of an anti-CD30 antibody-drug conjugate described herein. In some embodiments, the subject has plasma HIV RNA ≥500 copies/mL prior to administration of an anti-CD30 antibody-drug conjugate described herein. In some embodiments, the subject has plasma HIV RNA ≥500 copies/mL prior to receiving a first dose of an anti-CD30 antibody-drug conjugate described herein. In some embodiments, the subject has had plasma HIV RNA ≥500 copies/mL in a 3-month period prior to administration of an anti-CD30 antibody-drug conjugate described herein. In some embodiments, the subject has had plasma HIV RNA ≥500 copies/mL in a 3-month period prior to receiving a first dose of an anti-CD30 antibody-drug conjugate described herein. In some embodiments, the subject has had plasma HIV RNA ≥400 copies/mL in a 3-month period prior to administration of an anti-CD30 antibody-drug conjugate described herein. In some embodiments, the subject has had plasma HIV RNA ≥400 copies/mL in a 3-month period prior to receiving a first dose of an anti-CD30 antibody-drug conjugate described herein. In some embodiments, the subject has had plasma HIV RNA ≥300 copies/mL in a 3-month period prior to administration of an anti-CD30 antibody-drug conjugate described herein. In some embodiments, the subject has had plasma HIV RNA ≥300 copies/mL in a 3-month period prior to receiving a first dose of an anti-CD30 antibody-drug conjugate described herein. In some embodiments, the subject has had plasma HIV RNA ≥200 copies/mL in a 3-month period prior to administration of an anti-CD30 antibody-drug conjugate described herein. In some embodiments, the subject has had plasma HIV RNA ≥200 copies/mL in a 3-month period prior to receiving a first dose of an anti-CD30 antibody-drug conjugate described herein. In some embodiments, the subject has had plasma HIV RNA ≥100 copies/mL in a 3-month period prior to administration of an anti-CD30 antibody-drug conjugate described herein. In some embodiments, the subject has had plasma HIV RNA ≥100 copies/mL in a 3-month period prior to receiving a first dose of an anti-CD30 antibody-drug conjugate described herein. In some embodiments, the subject has had plasma HIV RNA ≤25 copies/mL in a 3-month period prior to administration of an anti-CD30 antibody-drug conjugate described herein. In some embodiments, the subject has had plasma HIV RNA ≤25 copies/mL in a 3-month period prior to receiving a first dose of an anti-CD30 antibody-drug conjugate described herein. In some embodiments, the subject has had plasma HIV RNA ≤25 copies/mL for at least 6 months prior to administration of an anti-CD30 antibody-drug conjugate described herein. In some embodiments, the subject has had plasma HIV RNA ≤25 copies/mL for at least 6 months prior to receiving a first dose of an anti-CD30 antibody-drug conjugate described herein. In some embodiments, the subject has had plasma HIV RNA ≤25 copies/mL for at least 12 months prior to administration of an anti-CD30 antibody-drug conjugate described herein. In some embodiments, the subject has had plasma HIV RNA ≤25 copies/mL for at least 12 months prior to receiving a first dose of an anti-CD30 antibody-drug conjugate described herein. In some embodiments, the subject has had plasma HIV RNA ≤25 copies/mL for at least 24 months prior to administration of an anti-CD30 antibody-drug conjugate described herein. In some embodiments, the subject has had plasma HIV RNA ≤25 copies/mL for at least 24 months prior to receiving a first dose of an anti-CD30 antibody-drug conjugate described herein. In some embodiments, the subject has had plasma HIV RNA ≤50 copies/mL in a 3-month period prior to administration of an anti-CD30 antibody-drug conjugate described herein. In some embodiments, the subject has had plasma HIV RNA ≤50 copies/mL in a 3-month period prior to receiving a first dose of an anti-CD30 antibody-drug conjugate described herein. In some embodiments, the subject has had plasma HIV RNA ≤50 copies/mL for at least 6 months prior to administration of an anti-CD30 antibody-drug conjugate described herein. In some embodiments, the subject has had plasma HIV RNA ≤50 copies/mL for at least 6 months prior to receiving a first dose of an anti-CD30 antibody-drug conjugate described herein. In some embodiments, the subject has had plasma HIV RNA ≤50 copies/mL for at least 12 months prior to administration of an anti-CD30 antibody-drug conjugate described herein. In some embodiments, the subject has had plasma HIV RNA ≤50 copies/mL for at least 12 months prior to receiving a first dose of an anti-CD30 antibody-drug conjugate described herein. In some embodiments, the subject has had plasma HIV RNA ≤50 copies/mL for at least 24 months prior to administration of an anti-CD30 antibody-drug conjugate described herein. In some embodiments, the subject has had plasma HIV RNA ≤50 copies/mL for at least 24 months prior to receiving a first dose of an anti-CD30 antibody-drug conjugate described herein. In some embodiments, the subject has had plasma HIV RNA ≤100 copies/mL in a 3-month period prior to administration of an anti-CD30 antibody-drug conjugate described herein. In some embodiments, the subject has had plasma HIV RNA ≤100 copies/mL in a 3-month period prior to receiving a first dose of an anti-CD30 antibody-drug conjugate described herein. In some embodiments, the subject has had plasma HIV RNA ≤100 copies/mL for at least 6 months prior to administration of an anti-CD30 antibody-drug conjugate described herein. In some embodiments, the subject has had plasma HIV RNA ≤100 copies/mL for at least 6 months prior to receiving a first dose of an anti-CD30 antibody-drug conjugate described herein. In some embodiments, the subject has had plasma HIV RNA ≤100 copies/mL for at least 12 months prior to administration of an anti-CD30 antibody-drug conjugate described herein. In some embodiments, the subject has had plasma HIV RNA ≤100 copies/mL for at least 12 months prior to receiving a first dose of an anti-CD30 antibody-drug conjugate described herein. In some embodiments, the subject has had plasma HIV RNA ≤100 copies/mL for at least 24 months prior to administration of an anti-CD30 antibody-drug conjugate described herein. In some embodiments, the subject has had plasma HIV RNA ≤100 copies/mL for at least 24 months prior to receiving a first dose of an anti-CD30 antibody-drug conjugate described herein. In some embodiments, the subject has had plasma HIV RNA ≤200 copies/mL in a 3-month period prior to administration of an anti-CD30 antibody-drug conjugate described herein. In some embodiments, the subject has had plasma HIV RNA ≤200 copies/mL in a 3-month period prior to receiving a first dose of an anti-CD30 antibody-drug conjugate described herein. In some embodiments, the subject has had plasma HIV RNA ≤200 copies/mL for at least 6 months prior to administration of an anti-CD30 antibody-drug conjugate described herein. In some embodiments, the subject has had plasma HIV RNA ≤200 copies/mL for at least 6 months prior to receiving a first dose of an anti-CD30 antibody-drug conjugate described herein. In some embodiments, the subject has had plasma HIV RNA ≤200 copies/mL for at least 12 months prior to administration of an anti-CD30 antibody-drug conjugate described herein. In some embodiments, the subject has had plasma HIV RNA ≤200 copies/mL for at least 12 months prior to receiving a first dose of an anti-CD30 antibody-drug conjugate described herein. In some embodiments, the subject has had plasma HIV RNA ≤200 copies/mL for at least 24 months prior to administration of an anti-CD30 antibody-drug conjugate described herein. In some embodiments, the subject has had plasma HIV RNA ≤200 copies/mL for at least 24 months prior to receiving a first dose of an anti-CD30 antibody-drug conjugate described herein.

In some embodiments, the subject has a life expectancy of greater than 12 months prior to administration of an anti-CD30 antibody-drug conjugate described herein. In some embodiments, the subject has a life expectancy of greater than 12 months prior to receiving a first dose of an anti-CD30 antibody-drug conjugate described herein. In some embodiments, the subject has a life expectancy of greater than 9 months prior to administration of an anti-CD30 antibody-drug conjugate described herein. In some embodiments, the subject has a life expectancy of greater than 9 months prior to receiving a first dose of an anti-CD30 antibody-drug conjugate described herein. In some embodiments, the subject has a life expectancy of greater than 6 months prior to administration of an anti-CD30 antibody-drug conjugate described herein. In some embodiments, the subject has a life expectancy of greater than 6 months prior to receiving a first dose of an anti-CD30 antibody-drug conjugate described herein. In some embodiments, the subject has a life expectancy of greater than 3 months prior to administration of an anti-CD30 antibody-drug conjugate described herein. In some embodiments, the subject has a life expectancy of greater than 3 months prior to receiving a first dose of an anti-CD30 antibody-drug conjugate described herein.

In some embodiments, the absolute neutrophil count in a sample from the subject is assessed prior to the administration, e.g., the first administration, of an anti-CD30 antibody-drug conjugate described here. In some embodiments, the subject has an absolute neutrophil count of ≥1000/mm₃ prior to administration of an anti-CD30 antibody-drug conjugate described herein. In some embodiments, the subject has an absolute neutrophil count of ≥1000/mm³ prior to receiving a first dose of an anti-CD30 antibody-drug conjugate described herein. In some embodiments, the subject has an absolute neutrophil count of ≥750/mm³ prior to administration of an anti-CD30 antibody-drug conjugate described herein. In some embodiments, the subject has an absolute neutrophil count of ≥750/mm³ prior to receiving a first dose of an anti-CD30 antibody-drug conjugate described herein. In some embodiments, the subject has an absolute neutrophil count of ≥500/mm³ prior to administration of an anti-CD30 antibody-drug conjugate described herein. In some embodiments, the subject has an absolute neutrophil count of >500/mm³ prior to receiving a first dose of an anti-CD30 antibody-drug conjugate described herein.

In some embodiments, the amount of hemoglobin in a sample from the subject is assessed prior to the administration, e.g., the first administration, of an anti-CD30 antibody-drug conjugate described here. In some embodiments, the subject is a male and has hemoglobin ≥12 gm/dL prior to administration of an anti-CD30 antibody-drug conjugate described herein. In some embodiments, the subject is a male and has hemoglobin ≥12 gm/dL prior to receiving a first dose of an anti-CD30 antibody-drug conjugate described herein. In some embodiments, the subject is a male and has hemoglobin ≥11.5 gm/dL prior to administration of an anti-CD30 antibody-drug conjugate described herein. In some embodiments, the subject is a male and has hemoglobin ≥11.5 gm/dL prior to receiving a first dose of an anti-CD30 antibody-drug conjugate described herein. In some embodiments, the subject is a male and has hemoglobin ≥11 gm/dL prior to administration of an anti-CD30 antibody-drug conjugate described herein. In some embodiments, the subject is a male and has hemoglobin ≥11 gm/dL prior to receiving a first dose of an anti-CD30 antibody-drug conjugate described herein. In some embodiments, the subject is a male and has hemoglobin ≥10.5 gm/dL prior to administration of an anti-CD30 antibody-drug conjugate described herein. In some embodiments, the subject is a male and has hemoglobin ≥10.5 gm/dL prior to receiving a first dose of an anti-CD30 antibody-drug conjugate described herein. In some embodiments, the subject is a male and has hemoglobin ≥10 gm/dL prior to administration of an anti-CD30 antibody-drug conjugate described herein. In some embodiments, the subject is a male and has hemoglobin ≥10 gm/dL prior to receiving a first dose of an anti-CD30 antibody-drug conjugate described herein. In some embodiments, the subject is a male and has hemoglobin ≥9.5 gm/dL prior to administration of an anti-CD30 antibody-drug conjugate described herein. In some embodiments, the subject is a male and has hemoglobin ≥9.5 gm/dL prior to receiving a first dose of an anti-CD30 antibody-drug conjugate described herein. In some embodiments, the subject is a male and has hemoglobin ≥9 gm/dL prior to administration of an anti-CD30 antibody-drug conjugate described herein. In some embodiments, the subject is a male and has hemoglobin ≥9 gm/dL prior to receiving a first dose of an anti-CD30 antibody-drug conjugate described herein. In some embodiments, the subject is a female and has hemoglobin ≥11 gm/dL prior to administration of an anti-CD30 antibody-drug conjugate described herein. In some embodiments, the subject is a female and has hemoglobin ≥11 gm/dL prior to receiving a first dose of an anti-CD30 antibody-drug conjugate described herein. In some embodiments, the subject is a female and has hemoglobin ≥10.5 gm/dL prior to administration of an anti-CD30 antibody-drug conjugate described herein. In some embodiments, the subject is a female and has hemoglobin ≥10.5 gm/dL prior to receiving a first dose of an anti-CD30 antibody-drug conjugate described herein. In some embodiments, the subject is a female and has hemoglobin 10 gm/dL prior to administration of an anti-CD30 antibody-drug conjugate described herein. In some embodiments, the subject is a female and has hemoglobin 10 gm/dL prior to receiving a first dose of an anti-CD30 antibody-drug conjugate described herein. In some embodiments, the subject is a female and has hemoglobin 9.5 gm/dL prior to administration of an anti-CD30 antibody-drug conjugate described herein. In some embodiments, the subject is a female and has hemoglobin 9.5 gm/dL prior to receiving a first dose of an anti-CD30 antibody-drug conjugate described herein. In some embodiments, the subject is a female and has hemoglobin ≥9 gm/dL prior to administration of an anti-CD30 antibody-drug conjugate described herein. In some embodiments, the subject is a female and has hemoglobin ≥9 gm/dL prior to receiving a first dose of an anti-CD30 antibody-drug conjugate described herein. In some embodiments, the subject is a female and has hemoglobin ≥8.5 gm/dL prior to administration of an anti-CD30 antibody-drug conjugate described herein. In some embodiments, the subject is a female and has hemoglobin ≥8.5 gm/dL prior to receiving a first dose of an anti-CD30 antibody-drug conjugate described herein. In some embodiments, the subject is a female and has hemoglobin ≥8 gm/dL prior to administration of an anti-CD30 antibody-drug conjugate described herein. In some embodiments, the subject is a female and has hemoglobin ≥8 gm/dL prior to receiving a first dose of an anti-CD30 antibody-drug conjugate described herein.

In some embodiments, the amount of serum alanine transaminase (SGPT/ALT) in a sample from the subject is assessed prior to the administration, e.g., the first administration, of an anti-CD30 antibody-drug conjugate described here. Serum alanine transaminase is also known as serum glutamate-pyruvate transaminase and serum glutamic-pyruvic transaminase. In some embodiments, the amount of SGPT/ALT is compared to the upper limit of normal (ULN). In some embodiments, the upper limit of normal for a male subject is 45 international units per liter (IU/L). In some embodiments, the upper limit of normal for a female subject is 34 IU/L. In some embodiments, the sample is a serum sample. In some embodiments, the subject has SGPT/ALT <3.0×ULN prior to administration of an anti-CD30 antibody-drug conjugate described herein. In some embodiments, the subject has SGPT/ALT <3.0×ULN prior to receiving a first dose of an anti-CD30 antibody-drug conjugate described herein. In some embodiments, the subject has SGPT/ALT <2.5×ULN prior to administration of an anti-CD30 antibody-drug conjugate described herein. In some embodiments, the subject has SGPT/ALT <2.5×ULN prior to receiving a first dose of an anti-CD30 antibody-drug conjugate described herein. In some embodiments, the subject has SGPT/ALT <2.0×ULN prior to administration of an anti-CD30 antibody-drug conjugate described herein. In some embodiments, the subject has SGPT/ALT <2.0×ULN prior to receiving a first dose of an anti-CD30 antibody-drug conjugate described herein. In some embodiments, the subject has SGPT/ALT <1.5×ULN prior to administration of an anti-CD30 antibody-drug conjugate described herein. In some embodiments, the subject has SGPT/ALT <1.5×ULN prior to receiving a first dose of an anti-CD30 antibody-drug conjugate described herein.

In some embodiments, the amount of serum aspartate transaminase (SGOT/AST) in a sample from the subject is assessed prior to the administration, e.g., the first administration, of an anti-CD30 antibody-drug conjugate described here. Serum aspartate transaminase is also known as aspartate transaminase (AST), aspartate aminotransferase, AspAT/ASAT/AAT, and (serum) glutamic oxaloacetic transaminase (GOT, SGOT). In some embodiments, the amount of SGOT/AST is compared to the upper limit of normal (ULN). In some embodiments, the upper limit of normal for a male subject is 40 international units per liter (IU/L). In some embodiments, the upper limit of normal for a female subject is 34 IU/L. In some embodiments, the sample is a serum sample. In some embodiments, the subject has SGOT/AST <3.0×ULN prior to administration of an anti-CD30 antibody-drug conjugate described herein. In some embodiments, the subject has SGOT/AST <3.0×ULN prior to receiving a first dose of an anti-CD30 antibody-drug conjugate described herein. In some embodiments, the subject has SGOT/AST <2.5×ULN prior to administration of an anti-CD30 antibody-drug conjugate described herein. In some embodiments, the subject has SGOT/AST <2.5×ULN prior to receiving a first dose of an anti-CD30 antibody-drug conjugate described herein. In some embodiments, the subject has SGOT/AST <2.0×ULN prior to administration of an anti-CD30 antibody-drug conjugate described herein. In some embodiments, the subject has SGOT/AST <2.0×ULN prior to receiving a first dose of an anti-CD30 antibody-drug conjugate described herein. In some embodiments, the subject has SGOT/AST <1.5×ULN prior to administration of an anti-CD30 antibody-drug conjugate described herein. In some embodiments, the subject has SGOT/AST <1.5×ULN prior to receiving a first dose of an anti-CD30 antibody-drug conjugate described herein.

In some embodiments, the amount of total bilirubin in a sample from the subject is assessed prior to the administration, e.g., the first administration, of an anti-CD30 antibody-drug conjugate described here. In some embodiments, the amount of total bilirubin is compared to the upper limit of normal (ULN). In some embodiments, the upper limit of normal for a subject is 1.2 mg/dL. In some embodiments, the sample is a serum sample. In some embodiments, the subject has total bilirubin <3.0×ULN prior to administration of an anti-CD30 antibody-drug conjugate described herein. In some embodiments, the subject has total bilirubin <3.0×ULN prior to receiving a first dose of an anti-CD30 antibody-drug conjugate described herein. In some embodiments, the subject has total bilirubin <2.5×ULN prior to administration of an anti-CD30 antibody-drug conjugate described herein. In some embodiments, the subject has total bilirubin <2.5×ULN prior to receiving a first dose of an anti-CD30 antibody-drug conjugate described herein. In some embodiments, the subject has total bilirubin <2.0×ULN prior to administration of an anti-CD30 antibody-drug conjugate described herein. In some embodiments, the subject has total bilirubin <2.0×ULN prior to receiving a first dose of an anti-CD30 antibody-drug conjugate described herein. In some embodiments, the subject has total bilirubin <1.5×ULN prior to administration of an anti-CD30 antibody-drug conjugate described herein. In some embodiments, the subject has total bilirubin <1.5×ULN prior to receiving a first dose of an anti-CD30 antibody-drug conjugate described herein.

In some embodiments, the amount of creatinine in a sample from the subject is assessed prior to the administration, e.g., the first administration, of an anti-CD30 antibody-drug conjugate described here. In some embodiments, the amount of creatinine is compared to the upper limit of normal (ULN). In some embodiments, the upper limit of normal for a male subject is 1.2 mg/dL. In some embodiments, the upper limit of normal for a female subject is 1.0 mg/dL. In some embodiments, the sample is a serum sample. In some embodiments, the subject has creatinine <2.0×ULN prior to administration of an anti-CD30 antibody-drug conjugate described herein. In some embodiments, the subject has creatinine <2.0×ULN prior to receiving a first dose of an anti-CD30 antibody-drug conjugate described herein. In some embodiments, the subject has creatinine <1.75×ULN prior to administration of an anti-CD30 antibody-drug conjugate described herein. In some embodiments, the subject has creatinine <1.75×ULN prior to receiving a first dose of an anti-CD30 antibody-drug conjugate described herein. In some embodiments, the subject has creatinine <1.5×ULN prior to administration of an anti-CD30 antibody-drug conjugate described herein. In some embodiments, the subject has creatinine <1.5×ULN prior to receiving a first dose of an anti-CD30 antibody-drug conjugate described herein. In some embodiments, the subject has creatinine <1.25×ULN prior to administration of an anti-CD30 antibody-drug conjugate described herein. In some embodiments, the subject has creatinine <1.25×ULN prior to receiving a first dose of an anti-CD30 antibody-drug conjugate described herein.

In some embodiments, the subject has received antiretroviral therapy (ART) prior to the administration, e.g., the first administration, of an anti-CD30 antibody-drug conjugate described here. In some embodiments, the subject received ART for at least 12 weeks prior to administration of an anti-CD30 antibody-drug conjugate described herein. In some embodiments, the subject received ART for at least 12 weeks prior to receiving a first dose of an anti-CD30 antibody-drug conjugate described herein. In some embodiments, the subject received ART for at least 16 weeks prior to administration of an anti-CD30 antibody-drug conjugate described herein. In some embodiments, the subject received ART for at least 16 weeks prior to receiving a first dose of an anti-CD30 antibody-drug conjugate described herein. In some embodiments, the subject received ART for at least 24 weeks prior to administration of an anti-CD30 antibody-drug conjugate described herein. In some embodiments, the subject received ART for at least 24 weeks months prior to receiving a first dose of an anti-CD30 antibody-drug conjugate described herein. In some embodiments, the subject received ART for at least 9 months prior to administration of an anti-CD30 antibody-drug conjugate described herein. In some embodiments, the subject received ART for at least 9 months prior to receiving a first dose of an anti-CD30 antibody-drug conjugate described herein. In some embodiments, the subject received ART for at least 12 months prior to administration of an anti-CD30 antibody-drug conjugate described herein. In some embodiments, the subject received ART for at least 12 months prior to receiving a first dose of an anti-CD30 antibody-drug conjugate described herein. In some embodiments, the subject received ART for at least 15 months prior to administration of an anti-CD30 antibody-drug conjugate described herein. In some embodiments, the subject received ART for at least 15 months prior to receiving a first dose of an anti-CD30 antibody-drug conjugate described herein. In some embodiments, the subject received ART for at least 18 months prior to administration of an anti-CD30 antibody-drug conjugate described herein. In some embodiments, the subject received ART for at least 18 months prior to receiving a first dose of an anti-CD30 antibody-drug conjugate described herein. In some embodiments, the subject received ART for at least 24 months prior to administration of an anti-CD30 antibody-drug conjugate described herein. In some embodiments, the subject received ART for at least 24 months prior to receiving a first dose of an anti-CD30 antibody-drug conjugate described herein. In some embodiments, the subject received ART for at least 36 months prior to administration of an anti-CD30 antibody-drug conjugate described herein. In some embodiments, the subject received ART for at least 36 months prior to receiving a first dose of an anti-CD30 antibody-drug conjugate described herein. In some embodiments, the subject received ART for at least 48 months prior to administration of an anti-CD30 antibody-drug conjugate described herein. In some embodiments, the subject received ART for at least 48 months prior to receiving a first dose of an anti-CD30 antibody-drug conjugate described herein. In some embodiments, the subject has maintained an HIV viral load of <25 copies/mL while on ART. In some embodiments, the subject has maintained an HIV viral load of <25 copies/mL for at least 6 months. In some embodiments, the subject has maintained an HIV viral load of <25 copies/mL for at least 12 months. In some embodiments, the subject has maintained an HIV viral load of <25 copies/mL for at least 24 months. In some embodiments, the subject has maintained an HIV viral load of <25 copies/mL for at least 36 months. In some embodiments, the subject has maintained an HIV viral load of <50 copies/mL while on ART. In some embodiments, the subject has maintained an HIV viral load of <50 copies/mL for at least 6 months. In some embodiments, the subject has maintained an HIV viral load of <50 copies/mL for at least 12 months. In some embodiments, the subject has maintained an HIV viral load of <50 copies/mL for at least 24 months. In some embodiments, the subject has maintained an HIV viral load of <50 copies/mL for at least 36 months. In some embodiments, the subject has maintained an HIV viral load of <100 copies/mL while on ART. In some embodiments, the subject has maintained an HIV viral load of <100 copies/mL for at least 6 months. In some embodiments, the subject has maintained an HIV viral load of <100 copies/mL for at least 12 months. In some embodiments, the subject has maintained an HIV viral load of <100 copies/mL for at least 24 months. In some embodiments, the subject has maintained an HIV viral load of <100 copies/mL for at least 36 months. In some embodiments, the subject has maintained an HIV viral load of <200 copies/mL while on ART. In some embodiments, the subject has maintained an HIV viral load of <200 copies/mL for at least 6 months. In some embodiments, the subject has maintained an HIV viral load of <200 copies/mL for at least 12 months. In some embodiments, the subject has maintained an HIV viral load of <200 copies/mL for at least 24 months. In some embodiments, the subject has maintained an HIV viral load of <200 copies/mL for at least 36 months.

In some embodiments, the antibody-drug conjugate is administered in combination with ART. In some embodiments, the ART is a nucleoside reverse transcriptase inhibitor, non-nucleoside reverse transcriptase inhibitor, protease inhibitor, fusion inhibitor, CCR5 antagonist, integrase inhibitor, post-attachment inhibitor, or pharmacokinetic enhancer. In some embodiments, the ART is a nucleoside reverse transcriptase inhibitor. In some embodiments, the ART is a non-nucleoside reverse transcriptase inhibitor. In some embodiments, the ART is a protease inhibitor. In some embodiments, the ART is a fusion inhibitor. In some embodiments, the ART is a CCR5 antagonist. In some embodiments, the ART is an integrase inhibitor. In some embodiments, the ART is a post-attachment inhibitor. In some embodiments, the ART is a pharmacokinetic enhancer. In some embodiments, the ART comprises two or more of a nucleoside reverse transcriptase inhibitor, non-nucleoside reverse transcriptase inhibitor, protease inhibitor, fusion inhibitor, CCR5 antagonist, integrase inhibitor, post-attachment inhibitor, and pharmacokinetic enhancer. In some embodiments, the ART comprises three or more of a nucleoside reverse transcriptase inhibitor, non-nucleoside reverse transcriptase inhibitor, protease inhibitor, fusion inhibitor, CCR5 antagonist, integrase inhibitor, post-attachment inhibitor, and pharmacokinetic enhancer. In some embodiments, the ART comprises four or more of a nucleoside reverse transcriptase inhibitor, non-nucleoside reverse transcriptase inhibitor, protease inhibitor, fusion inhibitor, CCR5 antagonist, integrase inhibitor, post-attachment inhibitor, and pharmacokinetic enhancer. In some embodiments, the ART comprises five or more of a nucleoside reverse transcriptase inhibitor, non-nucleoside reverse transcriptase inhibitor, protease inhibitor, fusion inhibitor, CCR5 antagonist, integrase inhibitor, post-attachment inhibitor, and pharmacokinetic enhancer. In some embodiments, the ART comprises one or more of abacavir, emtricitabine, lamivudine, tenofovir disoproxil fumarate, zidovudine, doravirine, efavirenz, etravirine, nevirapine, rilpivirine, atazanavir, darunavir, fosamprenavir, ritonavir, saquinavir, tipranavir, enfuvirtide, maraviroc, dolutegravir, raltegravir, ibalizumab, and cobicistat. In some embodiments, the ART comprises abacavir. In some embodiments, the ART comprises emtricitabine. In some embodiments, the ART comprises lamivudine. In some embodiments, the ART comprises tenofovir disoproxil fumarate. In some embodiments, the ART comprises zidovudine. In some embodiments, the ART comprises doravirine. In some embodiments, the ART comprises efavirenz. In some embodiments, the ART comprises etravirine. In some embodiments, the ART comprises nevirapine. In some embodiments, the ART comprises rilpivirine. In some embodiments, the ART comprises atazanavir. In some embodiments, the ART comprises darunavir. In some embodiments, the ART comprises fosamprenavir. In some embodiments, the ART comprises ritonavir. In some embodiments, the ART comprises saquinavir. In some embodiments, the ART comprises tipranavir. In some embodiments, the ART comprises enfuvirtide. In some embodiments, the ART comprises maraviroc. In some embodiments, the ART comprises dolutegravir. In some embodiments, the ART comprises raltegravir. In some embodiments, the ART comprises ibalizumab. In some embodiments, the ART comprises cobicistat. In some embodiments the ART comprises two or more of abacavir, emtricitabine, lamivudine, tenofovir disoproxil fumarate, zidovudine, doravirine, efavirenz, etravirine, nevirapine, rilpivirine, atazanavir, darunavir, fosamprenavir, ritonavir, saquinavir, tipranavir, enfuvirtide, maraviroc, dolutegravir, raltegravir, ibalizumab, and cobicistat. In some embodiments the ART comprises three or more of abacavir, emtricitabine, lamivudine, tenofovir disoproxil fumarate, zidovudine, doravirine, efavirenz, etravirine, nevirapine, rilpivirine, atazanavir, darunavir, fosamprenavir, ritonavir, saquinavir, tipranavir, enfuvirtide, maraviroc, dolutegravir, raltegravir, ibalizumab, and cobicistat. In some embodiments the ART comprises four or more of abacavir, emtricitabine, lamivudine, tenofovir disoproxil fumarate, zidovudine, doravirine, efavirenz, etravirine, nevirapine, rilpivirine, atazanavir, darunavir, fosamprenavir, ritonavir, saquinavir, tipranavir, enfuvirtide, maraviroc, dolutegravir, raltegravir, ibalizumab, and cobicistat. In some embodiments the ART comprises five or more of abacavir, emtricitabine, lamivudine, tenofovir disoproxil fumarate, zidovudine, doravirine, efavirenz, etravirine, nevirapine, rilpivirine, atazanavir, darunavir, fosamprenavir, ritonavir, saquinavir, tipranavir, enfuvirtide, maraviroc, dolutegravir, raltegravir, ibalizumab, and cobicistat. In some embodiments, the ART does not comprise a strong CYP3A4 inhibitor. In some embodiments, the ART does not comprise a strong P-gp inhibitor. In some embodiments, the ART does not comprise cobicistat. In some embodiments, the ART does not comprise ritonavir.

B. Routes of Administration

An anti-CD30 antibody, antigen-binding fragment thereof, or antibody-drug conjugate described herein can be administered by any suitable route and mode. Suitable routes of administering antibodies and/or antibody-drug conjugate of the present invention are well known in the art and may be selected by those of ordinary skill in the art. In one embodiment, the anti-CD30 antibody, antigen-binding fragment thereof, or antibody-drug conjugate described herein is administered parenterally. Parenteral administration refers to modes of administration other than enteral and topical administration, usually by injection, and include epidermal, intravenous, intramuscular, intraarterial, intrathecal, intracapsular, intraorbital, intracardiac, intradermal, intraperitoneal, intratendinous, transtracheal, subcutaneous, subcuticular, intraarticular, subcapsular, subarachnoid, intraspinal, intracranial, intrathoracic, epidural and intrasternal injection and infusion. In some embodiments, the route of administration of an anti-CD30 antibody, antigen-binding fragment thereof, or antibody-drug conjugate described herein described herein is intravenous injection or infusion. In some embodiments, the route of administration of an anti-CD30 antibody, antigen-binding fragment thereof, or antibody-drug conjugate described herein is intravenous infusion. In some embodiments, the intravenous infusion is about a 15-minute to about a 2-hour infusion. In some embodiments, the intravenous infusion is about a 30-minute infusion. In some embodiments, the intravenous infusion is about a 60-minute infusion. In some embodiments, the intravenous infusion is a 30-minute infusion. In some embodiments, the intravenous infusion is a 60-minute infusion.

C. Dosage and Frequency of Administration

In one aspect, the present invention provides for methods of treating a subject with an HIV infection as described herein with a particular dose of an anti-CD30 antibody, antigen-binding fragment thereof, or antibody-drug conjugate described herein, wherein the subject is administered the antibody, antigen-binding fragment thereof, or antibody-drug conjugate described herein with particular frequencies.

In one embodiment of the methods or uses or product for uses provided herein, an anti-CD30 antibody-drug conjugate or antigen-binding fragment thereof as described herein is administered to the subject at a dose ranging from about 0.05 mg/kg to about 1.3 mg/kg of the subject's body weight. In certain embodiments, the dose is about 0.05 mg/kg, about 0.10 mg/kg, about 0.15 mg/kg, about 0.20 mg/kg, about 0.25 mg/kg, about 0.30 mg/kg, about 0.35 mg/kg, about 0.40 mg/kg, about 0.45 mg/kg, about 0.50 mg/kg, about 0.55 mg/kg, about 0.60 mg/kg, about 0.65 mg/kg, about 0.70 mg/kg, about 0.75 mg/kg, about 0.80 mg/kg, about 0.85 mg/kg, about 0.90 mg/kg, about 0.95 mg/kg, about 1.0 mg/kg, about 1.05 mg/kg, about 1.1 mg/kg, about 1.15 mg/kg, about 1.2 mg/kg, about 1.25 mg/kg, or about 1.3 mg/kg of the subject's body weight. In some embodiments, the dose is about 0.3 mg/kg to about 0.9 mg/kg of the subject's body weight. In some embodiments, the dose is about 0.9 mg/kg to about 1.2 mg/kg of the subject's body weight. In one embodiment, the dose is about 0.30 mg/kg of the subject's body weight. In one embodiment, the dose is about 0.60 mg/kg of the subject's body weight. In one embodiment, the dose is about 0.90 mg/kg of the subject's body weight. In one embodiment, the dose is about 1.2 mg/kg of the subject's body weight. In certain embodiments, the dose is 0.05 mg/kg, 0.10 mg/kg, 0.15 mg/kg, 0.20 mg/kg, 0.25 mg/kg, 0.30 mg/kg, 0.35 mg/kg, 0.40 mg/kg, 0.45 mg/kg, 0.50 mg/kg, 0.55 mg/kg, 0.60 mg/kg, 0.65 mg/kg, 0.70 mg/kg, 0.75 mg/kg, 0.80 mg/kg, 0.85 mg/kg, 0.90 mg/kg, 0.95 mg/kg, 1.0 mg/kg, 1.05 mg/kg, 1.10 mg/kg, 1.15 mg/kg, 1.20 mg/kg, 1.25 mg/kg, or 1.30 mg/kg of the subject's body weight. In some embodiments, the dose is 0.30 mg/kg to 0.90 mg/kg of the subject's body weight. In some embodiments, the dose is 0.90 mg/kg to 1.2 mg/kg of the subject's body weight. In one embodiment, the dose is 0.30 mg/kg of the subject's body weight. In one embodiment, the dose is 0.60 mg/kg of the subject's body weight. In one embodiment, the dose is 0.90 mg/kg of the subject's body weight. In one embodiment, the dose is 1.2 mg/kg of the subject's body weight. In some embodiments, the dose is 0.30 mg/kg of the subject's body weight and the anti-CD30 antibody-drug conjugate is brentuximab vedotin. In some embodiments, the dose is 0.60 mg/kg of the subject's body weight and the anti-CD30 antibody-drug conjugate is brentuximab vedotin. In some embodiments, the dose is 0.90 mg/kg of the subject's body weight and the anti-CD30 antibody-drug conjugate is brentuximab vedotin. In some embodiments, the dose is 1.2 mg/kg of the subject's body weight and the anti-CD30 antibody-drug conjugate is brentuximab vedotin.

In one embodiment of the methods or uses or product for uses provided herein, an anti-CD30 antibody-drug conjugate or antigen-binding fragment thereof as described herein is administered to the subject once about every 1 to 4 weeks. In certain embodiments, an anti-CD30 antibody-drug conjugate or antigen-binding fragment thereof as described herein is administered once about every 1 week, once about every 2 weeks, once about every 3 weeks or once about every 4 weeks. In one embodiment, an anti-CD30 antibody-drug conjugate or antigen-binding fragment thereof as described herein is administered once about every 3 weeks. In one embodiment, an anti-CD30 antibody-drug conjugate or antigen-binding fragment thereof as described herein is administered once every 3 weeks. In some embodiments, the dose is about 0.05 mg/kg and is administered once about every 1 week. In some embodiments, the dose is about 0.05 mg/kg and is administered once about every 2 weeks. In some embodiments, the dose is about 0.05 mg/kg and is administered once about every 3 weeks. In some embodiments, the dose is about 0.05 mg/kg and is administered once about every 4 weeks. In some embodiments, the dose is about 0.10 mg/kg and is administered once about every 1 week. In some embodiments, the dose is about 0.10 mg/kg and is administered once about every 2 weeks. In some embodiments, the dose is about 0.10 mg/kg and is administered once about every 3 weeks. In some embodiments, the dose is about 0.10 mg/kg and is administered once about every 4 weeks. In some embodiments, the dose is about 0.15 mg/kg and is administered once about every 1 week. In some embodiments, the dose is about 0.15 mg/kg and is administered once about every 2 weeks. In some embodiments, the dose is about 0.15 mg/kg and is administered once about every 3 weeks. In some embodiments, the dose is about 0.15 mg/kg and is administered once about every 4 weeks. In some embodiments, the dose is about 0.20 mg/kg and is administered once about every 1 week. In some embodiments, the dose is about 0.20 mg/kg and is administered once about every 2 weeks. In some embodiments, the dose is about 0.20 mg/kg and is administered once about every 3 weeks. In some embodiments, the dose is about 0.20 mg/kg and is administered once about every 4 weeks. In some embodiments, the dose is about 0.25 mg/kg and is administered once about every 1 week. In some embodiments, the dose is about 0.25 mg/kg and is administered once about every 2 weeks. In some embodiments, the dose is about 0.25 mg/kg and is administered once about every 3 weeks. In some embodiments, the dose is about 0.25 mg/kg and is administered once about every 4 weeks. In some embodiments, the dose is about 0.30 mg/kg and is administered once about every 1 week. In some embodiments, the dose is about 0.30 mg/kg and is administered once about every 2 weeks. In some embodiments, the dose is about 0.30 mg/kg and is administered once about every 3 weeks. In some embodiments, the dose is about 0.30 mg/kg and is administered once about every 4 weeks. In some embodiments, the dose is about 0.35 mg/kg and is administered once about every 1 week. In some embodiments, the dose is about 0.35 mg/kg and is administered once about every 2 weeks. In some embodiments, the dose is about 0.35 mg/kg and is administered once about every 3 weeks. In some embodiments, the dose is about 0.35 mg/kg and is administered once about every 4 weeks. In some embodiments, the dose is about 0.40 mg/kg and is administered once about every 1 week. In some embodiments, the dose is about 0.40 mg/kg and is administered once about every 2 weeks. In some embodiments, the dose is about 0.40 mg/kg and is administered once about every 3 weeks. In some embodiments, the dose is about 0.40 mg/kg and is administered once about every 4 weeks. In some embodiments, the dose is about 0.45 mg/kg and is administered once about every 1 week. In some embodiments, the dose is about 0.45 mg/kg and is administered once about every 2 weeks. In some embodiments, the dose is about 0.45 mg/kg and is administered once about every 3 weeks. In some embodiments, the dose is about 0.45 mg/kg and is administered once about every 4 weeks. In some embodiments, the dose is about 0.50 mg/kg and is administered once about every 1 week. In some embodiments, the dose is about 0.50 mg/kg and is administered once about every 2 weeks. In some embodiments, the dose is about 0.50 mg/kg and is administered once about every 3 weeks. In some embodiments, the dose is about 0.50 mg/kg and is administered once about every 4 weeks. In some embodiments, the dose is about 0.55 mg/kg and is administered once about every 1 week. In some embodiments, the dose is about 0.55 mg/kg and is administered once about every 2 weeks. In some embodiments, the dose is about 0.55 mg/kg and is administered once about every 3 weeks. In some embodiments, the dose is about 0.55 mg/kg and is administered once about every 4 weeks. In some embodiments, the dose is about 0.60 mg/kg and is administered once about every 1 week. In some embodiments, the dose is about 0.60 mg/kg and is administered once about every 2 weeks. In some embodiments, the dose is about 0.60 mg/kg and is administered once about every 3 weeks. In some embodiments, the dose is about 0.60 mg/kg and is administered once about every 4 weeks. In some embodiments, the dose is about 0.65 mg/kg and is administered once about every 1 week. In some embodiments, the dose is about 0.65 mg/kg and is administered once about every 2 weeks. In some embodiments, the dose is about 0.65 mg/kg and is administered once about every 3 weeks. In some embodiments, the dose is about 0.65 mg/kg and is administered once about every 4 weeks. In some embodiments, the dose is about 0.70 mg/kg and is administered once about every 1 week. In some embodiments, the dose is about 0.70 mg/kg and is administered once about every 2 weeks. In some embodiments, the dose is about 0.70 mg/kg and is administered once about every 3 weeks. In some embodiments, the dose is about 0.70 mg/kg and is administered once about every 4 weeks. In some embodiments, the dose is about 0.75 mg/kg and is administered once about every 1 week. In some embodiments, the dose is about 0.75 mg/kg and is administered once about every 2 weeks. In some embodiments, the dose is about 0.75 mg/kg and is administered once about every 3 weeks. In some embodiments, the dose is about 0.75 mg/kg and is administered once about every 4 weeks. In some embodiments, the dose is about 0.80 mg/kg and is administered once about every 1 week. In some embodiments, the dose is about 0.80 mg/kg and is administered once about every 2 weeks. In some embodiments, the dose is about 0.80 mg/kg and is administered once about every 3 weeks. In some embodiments, the dose is about 0.80 mg/kg and is administered once about every 4 weeks. In some embodiments, the dose is about 0.85 mg/kg and is administered once about every 1 week. In some embodiments, the dose is about 0.85 mg/kg and is administered once about every 2 weeks. In some embodiments, the dose is about 0.85 mg/kg and is administered once about every 3 weeks. In some embodiments, the dose is about 0.85 mg/kg and is administered once about every 4 weeks. In some embodiments, the dose is about 0.90 mg/kg and is administered once about every 1 week. In some embodiments, the dose is about 0.90 mg/kg and is administered once about every 2 weeks. In some embodiments, the dose is about 0.90 mg/kg and is administered once about every 3 weeks. In some embodiments, the dose is about 0.90 mg/kg and is administered once about every 4 weeks. In some embodiments, the dose is about 1.0 mg/kg and is administered once about every 1 week. In some embodiments, the dose is about 1.0 mg/kg and is administered once about every 2 weeks. In some embodiments, the dose is about 1.0 mg/kg and is administered once about every 3 weeks. In some embodiments, the dose is about 1.0 mg/kg and is administered once about every 4 weeks. In some embodiments, the dose is about 1.05 mg/kg and is administered once about every 1 week. In some embodiments, the dose is about 1.05 mg/kg and is administered once about every 2 weeks. In some embodiments, the dose is about 1.05 mg/kg and is administered once about every 3 weeks. In some embodiments, the dose is about 1.05 mg/kg and is administered once about every 4 weeks. In some embodiments, the dose is about 1.10 mg/kg and is administered once about every 1 week. In some embodiments, the dose is about 1.10 mg/kg and is administered once about every 2 weeks. In some embodiments, the dose is about 1.10 mg/kg and is administered once about every 3 weeks. In some embodiments, the dose is about 1 10 mg/kg and is administered once about every 4 weeks. In some embodiments, the dose is about 1.15 mg/kg and is administered once about every 1 week. In some embodiments, the dose is about 1.15 mg/kg and is administered once about every 2 weeks. In some embodiments, the dose is about 1.15 mg/kg and is administered once about every 3 weeks. In some embodiments, the dose is about 1.15 mg/kg and is administered once about every 4 weeks. In some embodiments, the dose is about 1.20 mg/kg and is administered once about every 1 week. In some embodiments, the dose is about 1.20 mg/kg and is administered once about every 2 weeks. In some embodiments, the dose is about 1.20 mg/kg and is administered once about every 3 weeks. In some embodiments, the dose is about 1.20 mg/kg and is administered once about every 4 weeks. In some embodiments, the dose is about 1.25 mg/kg and is administered once about every 1 week. In some embodiments, the dose is about 1.25 mg/kg and is administered once about every 2 weeks. In some embodiments, the dose is about 1.25 mg/kg and is administered once about every 3 weeks. In some embodiments, the dose is about 1.25 mg/kg and is administered once about every 4 weeks. In some embodiments, the dose is about 1.30 mg/kg and is administered once about every 1 week. In some embodiments, the dose is about 1.30 mg/kg and is administered once about every 2 weeks. In some embodiments, the dose is about 1.30 mg/kg and is administered once about every 3 weeks. In some embodiments, the dose is about 1.30 mg/kg and is administered once about every 4 weeks. In some embodiments, the dose is 0.05 mg/kg and is administered once about every 1 week. In some embodiments, the dose is 0.05 mg/kg and is administered once about every 2 weeks. In some embodiments, the dose is 0.05 mg/kg and is administered once about every 3 weeks. In some embodiments, the dose is 0.05 mg/kg and is administered once about every 4 weeks. In some embodiments, the dose is 0.10 mg/kg and is administered once about every 1 week. In some embodiments, the dose is 0.10 mg/kg and is administered once about every 2 weeks. In some embodiments, the dose is 0.10 mg/kg and is administered once about every 3 weeks. In some embodiments, the dose is 0.10 mg/kg and is administered once about every 4 weeks. In some embodiments, the dose is 0.15 mg/kg and is administered once about every 1 week. In some embodiments, the dose is 0.15 mg/kg and is administered once about every 2 weeks. In some embodiments, the dose is 0.15 mg/kg and is administered once about every 3 weeks. In some embodiments, the dose is 0.15 mg/kg and is administered once about every 4 weeks. In some embodiments, the dose is 0.20 mg/kg and is administered once about every 1 week. In some embodiments, the dose is 0.20 mg/kg and is administered once about every 2 weeks. In some embodiments, the dose is 0.20 mg/kg and is administered once about every 3 weeks. In some embodiments, the dose is 0.20 mg/kg and is administered once about every 4 weeks. In some embodiments, the dose is 0.25 mg/kg and is administered once about every 1 week. In some embodiments, the dose is 0.25 mg/kg and is administered once about every 2 weeks. In some embodiments, the dose is 0.25 mg/kg and is administered once about every 3 weeks. In some embodiments, the dose is 0.25 mg/kg and is administered once about every 4 weeks. In some embodiments, the dose is 0.30 mg/kg and is administered once about every 1 week. In some embodiments, the dose is 0.30 mg/kg and is administered once about every 2 weeks. In some embodiments, the dose is 0.30 mg/kg and is administered once about every 3 weeks. In some embodiments, the dose is 0.30 mg/kg and is administered once about every 4 weeks. In some embodiments, the dose is 0.35 mg/kg and is administered once about every 1 week. In some embodiments, the dose is 0.35 mg/kg and is administered once about every 2 weeks. In some embodiments, the dose is 0.35 mg/kg and is administered once about every 3 weeks. In some embodiments, the dose is 0.35 mg/kg and is administered once about every 4 weeks. In some embodiments, the dose is 0.40 mg/kg and is administered once about every 1 week. In some embodiments, the dose is 0.40 mg/kg and is administered once about every 2 weeks. In some embodiments, the dose is 0.40 mg/kg and is administered once about every 3 weeks. In some embodiments, the dose is 0.40 mg/kg and is administered once about every 4 weeks. In some embodiments, the dose is 0.45 mg/kg and is administered once about every 1 week. In some embodiments, the dose is 0.45 mg/kg and is administered once about every 2 weeks. In some embodiments, the dose is 0.45 mg/kg and is administered once about every 3 weeks. In some embodiments, the dose is 0.45 mg/kg and is administered once about every 4 weeks. In some embodiments, the dose is 0.50 mg/kg and is administered once about every 1 week. In some embodiments, the dose is 0.50 mg/kg and is administered once about every 2 weeks. In some embodiments, the dose is 0.50 mg/kg and is administered once about every 3 weeks. In some embodiments, the dose is 0.50 mg/kg and is administered once about every 4 weeks. In some embodiments, the dose is 0.55 mg/kg and is administered once about every 1 week. In some embodiments, the dose is 0.55 mg/kg and is administered once about every 2 weeks. In some embodiments, the dose is 0.55 mg/kg and is administered once about every 3 weeks. In some embodiments, the dose is 0.55 mg/kg and is administered once about every 4 weeks. In some embodiments, the dose is 0.60 mg/kg and is administered once about every 1 week. In some embodiments, the dose is 0.60 mg/kg and is administered once about every 2 weeks. In some embodiments, the dose is 0.60 mg/kg and is administered once about every 3 weeks. In some embodiments, the dose is 0.60 mg/kg and is administered once about every 4 weeks. In some embodiments, the dose is 0.65 mg/kg and is administered once about every 1 week. In some embodiments, the dose is 0.65 mg/kg and is administered once about every 2 weeks. In some embodiments, the dose is 0.65 mg/kg and is administered once about every 3 weeks. In some embodiments, the dose is 0.65 mg/kg and is administered once about every 4 weeks. In some embodiments, the dose is 0.70 mg/kg and is administered once about every 1 week. In some embodiments, the dose is 0.70 mg/kg and is administered once about every 2 weeks. In some embodiments, the dose is 0.70 mg/kg and is administered once about every 3 weeks. In some embodiments, the dose is 0.70 mg/kg and is administered once about every 4 weeks. In some embodiments, the dose is 0.75 mg/kg and is administered once about every 1 week. In some embodiments, the dose is 0.75 mg/kg and is administered once about every 2 weeks. In some embodiments, the dose is 0.75 mg/kg and is administered once about every 3 weeks. In some embodiments, the dose is 0.75 mg/kg and is administered once about every 4 weeks. In some embodiments, the dose is 0.80 mg/kg and is administered once about every 1 week. In some embodiments, the dose is 0.80 mg/kg and is administered once about every 2 weeks. In some embodiments, the dose is 0.80 mg/kg and is administered once about every 3 weeks. In some embodiments, the dose is 0.80 mg/kg and is administered once about every 4 weeks. In some embodiments, the dose is 0.85 mg/kg and is administered once about every 1 week. In some embodiments, the dose is 0.85 mg/kg and is administered once about every 2 weeks. In some embodiments, the dose is 0.85 mg/kg and is administered once about every 3 weeks. In some embodiments, the dose is 0.85 mg/kg and is administered once about every 4 weeks. In some embodiments, the dose is 0.90 mg/kg and is administered once about every 1 week. In some embodiments, the dose is 0.90 mg/kg and is administered once about every 2 weeks. In some embodiments, the dose is 0.90 mg/kg and is administered once about every 3 weeks. In some embodiments, the dose is 0.90 mg/kg and is administered once about every 4 weeks. In some embodiments, the dose is 1.0 mg/kg and is administered once about every 1 week. In some embodiments, the dose is 1.0 mg/kg and is administered once about every 2 weeks. In some embodiments, the dose is 1.0 mg/kg and is administered once about every 3 weeks. In some embodiments, the dose is 1.0 mg/kg and is administered once about every 4 weeks. In some embodiments, the dose is 1.05 mg/kg and is administered once about every 1 week. In some embodiments, the dose is 1.05 mg/kg and is administered once about every 2 weeks. In some embodiments, the dose is 1.05 mg/kg and is administered once about every 3 weeks. In some embodiments, the dose is 1.05 mg/kg and is administered once about every 4 weeks. In some embodiments, the dose is 1.10 mg/kg and is administered once about every 1 week. In some embodiments, the dose is 1.10 mg/kg and is administered once about every 2 weeks. In some embodiments, the dose is 1.10 mg/kg and is administered once about every 3 weeks. In some embodiments, the dose is 1.10 mg/kg and is administered once about every 4 weeks. In some embodiments, the dose is 1.15 mg/kg and is administered once about every 1 week. In some embodiments, the dose is 1.15 mg/kg and is administered once about every 2 weeks. In some embodiments, the dose is 1.15 mg/kg and is administered once about every 3 weeks. In some embodiments, the dose is 1.15 mg/kg and is administered once about every 4 weeks. In some embodiments, the dose is 1.20 mg/kg and is administered once about every 1 week. In some embodiments, the dose is 1.20 mg/kg and is administered once about every 2 weeks. In some embodiments, the dose is 1.20 mg/kg and is administered once about every 3 weeks. In some embodiments, the dose is 1.20 mg/kg and is administered once about every 4 weeks. In some embodiments, the dose is 1.25 mg/kg and is administered once about every 1 week. In some embodiments, the dose is 1.25 mg/kg and is administered once about every 2 weeks. In some embodiments, the dose is 1.25 mg/kg and is administered once about every 3 weeks. In some embodiments, the dose is 1.25 mg/kg and is administered once about every 4 weeks. In some embodiments, the dose is 1.30 mg/kg and is administered once about every 1 week. In some embodiments, the dose is 1.30 mg/kg and is administered once about every 2 weeks. In some embodiments, the dose is 1.30 mg/kg and is administered once about every 3 weeks. In some embodiments, the dose is 1.30 mg/kg and is administered once about every 4 weeks. In some embodiments, the dose is about 0.30 mg/kg and is administered once about every 3 weeks (e.g., f 3 days). In some embodiments, the dose is about 0.30 mg/kg and is administered once every 3 weeks. In some embodiments, the dose is 0.30 mg/kg and is administered once every 3 weeks and the antibody-drug conjugate is brentuximab vedotin. In some embodiments, the dose is 0.30 mg/kg and is administered once about every 3 weeks (e.g., ±3 days). In some embodiments, the dose is 0.30 mg/kg and is administered once every 3 weeks. In some embodiments, the dose is 0.30 mg/kg and is administered once every 3 weeks and the antibody-drug conjugate is brentuximab vedotin. In some embodiments, the dose is about 0.60 mg/kg and is administered once about every 3 weeks (e.g., ±3 days). In some embodiments, the dose is about 0.60 mg/kg and is administered once every 3 weeks. In some embodiments, the dose is 0.60 mg/kg and is administered once every 3 weeks and the antibody-drug conjugate is brentuximab vedotin. In some embodiments, the dose is 0.60 mg/kg and is administered once about every 3 weeks (e.g., ±3 days). In some embodiments, the dose is 0.60 mg/kg and is administered once every 3 weeks. In some embodiments, the dose is 0.60 mg/kg and is administered once every 3 weeks and the antibody-drug conjugate is brentuximab vedotin. In some embodiments, the dose is about 0.90 mg/kg and is administered once about every 3 weeks (e.g., +3 days). In some embodiments, the dose is about 0.90 mg/kg and is administered once every 3 weeks. In some embodiments, the dose is 0.90 mg/kg and is administered once every 3 weeks and the antibody-drug conjugate is brentuximab vedotin. In some embodiments, the dose is 0.90 mg/kg and is administered once about every 3 weeks (e.g., ±3 days). In some embodiments, the dose is 0.90 mg/kg and is administered once every 3 weeks. In some embodiments, the dose is 0.90 mg/kg and is administered once every 3 weeks and the antibody-drug conjugate is brentuximab vedotin. In some embodiments, the dose is about 0.90 mg/kg and is administered once about every 2 weeks (e.g., ±2 days). In some embodiments, the dose is about 0.90 mg/kg and is administered once every 2 weeks. In some embodiments, the dose is about 0.90 mg/kg and is administered once every 2 weeks and the antibody-drug conjugate is brentuximab vedotin. In some embodiments, the dose is 0.90 mg/kg and is administered once about every 2 weeks (e.g., ±2 days). In some embodiments, the dose is 0.90 mg/kg and is administered once every 2 weeks. In some embodiments, the dose is 0.90 mg/kg and is administered once every 2 weeks and the antibody-drug conjugate is brentuximab vedotin. In some embodiments, the dose is about 1.20 mg/kg and is administered once about every 2 weeks (e.g., ±2 days). In some embodiments, the dose is about 1.20 mg/kg and is administered once every 2 weeks. In some embodiments, the dose is about 1.20 mg/kg and is administered once every 2 weeks and the antibody-drug conjugate is brentuximab vedotin. In some embodiments, the dose is 1.20 mg/kg and is administered once about every 2 weeks (e.g., ±2 days). In some embodiments, the dose is 1.20 mg/kg and is administered once every 2 weeks. In some embodiments, the dose is 1.20 mg/kg and is administered once every 2 weeks and the antibody-drug conjugate is brentuximab vedotin. The present invention encompasses embodiments wherein the subject is administered an antibody-drug conjugate or antigen-binding fragment thereof as described herein once in about a 3-week treatment cycle for at least 2, 3, 4, 5, 6, 7, 8, 9, 10, 11, 12 or more 3-week treatment cycles. In another embodiment, the subject is administered an antibody-drug conjugate or antigen-binding fragment thereof as described herein for between 2 and 48 3-week treatment cycles, such as between 2 and 36 cycles, such as between 2 and 24 cycles, such as between 2 and 15 cycles, such as between 2 and 12 cycles, such as 2 cycles, 3 cycles, 4 cycles, 5 cycles, 6 cycles, 7 cycles, 8 cycles, 9 cycles, 10 cycles, 11 cycles or 12 cycles. In some embodiments, the subject is administered an antibody-drug conjugate or antigen-binding fragment thereof as described herein for 12 cycles or more, such as 16 cycles or more, such as 24 cycles or more, such as 36 cycles or more. The present invention encompasses embodiments wherein the subject is administered an antibody-drug conjugate or antigen-binding fragment thereof as described herein once in about a 2-week treatment cycle for at least 2, 3, 4, 5, 6, 7, 8, 9, 10, 11, 12 or more 2-week treatment cycles. In another embodiment, the subject is administered an antibody-drug conjugate or antigen-binding fragment thereof as described herein for between 2 and 48 2-week treatment cycles, such as between 2 and 36 cycles, such as between 2 and 24 cycles, such as between 2 and 15 cycles, such as between 2 and 12 cycles, such as 2 cycles, 3 cycles, 4 cycles, 5 cycles, 6 cycles, 7 cycles, 8 cycles, 9 cycles, 10 cycles, 11 cycles or 12 cycles. In some embodiments, the subject is administered an antibody-drug conjugate or antigen-binding fragment thereof as described herein for 12 cycles or more, such as 16 cycles or more, such as 24 cycles or more, such as 36 cycles or more. The number of treatment cycles suitable for any specific subject or group of subjects may be determined by a person of skill in the art, typically a physician.

In one embodiment of the methods or uses or product for uses provided herein, an anti-CD30 antibody-drug conjugate or antigen-binding fragment thereof as described herein is administered to the subject at a flat dose ranging from about 50 mg to about 200 mg such as at a flat dose of about 50 mg or a flat dose of about 60 mg or a flat dose of about 70 mg or a flat dose of about 80 mg or a flat dose of about 90 mg or a flat dose of about 100 mg or a flat dose of about 110 mg or a flat dose of about 120 mg or a flat dose of about 130 mg or a flat dose of about 140 mg or a flat dose of about 150 mg or a flat dose of about 160 mg or a flat dose of about 170 mg or a flat dose of about 180 mg or a flat dose of about 190 mg or a flat dose of about 200 mg. In some embodiments, the flat dose is administered to the subject once about every 1 to 4 weeks. In certain embodiments, the flat dose is administered to the subject once about every 1 week, once about every 2 weeks, once about every 3 weeks or once about every 4 weeks. In some embodiments, the flat dose is administered to the subject once about every 3 weeks (e.g., ±3 days). In some embodiments, the flat dose is administered to the subject once every 3 weeks. In some embodiments, the flat dose is administered to the subject once every 3 weeks and the antibody-drug conjugate is brentuximab vedotin. In some embodiments, the flat does is administered to the subject once about every week (e.g., +1 day). In some embodiments, the flat does is administered to the subject once every week. In some embodiments, the flat dose is administered to the subject once about every 1 week for 3 consecutive weeks followed by about a 1 week rest period without any administration of the anti-CD30 antibody-drug conjugate or antigen-binding fragment thereof so that each cycle time is about 28 days including the resting period. In some embodiments, the flat dose is administered to the subject once every 1 week for 3 consecutive weeks followed by a 1 week rest period without any administration of the anti-CD30 antibody-drug conjugate or antigen-binding fragment thereof so that each cycle time is 28 days including the resting period. In some embodiments, the flat dose is administered to the subject on about days 1, 8, and 15 of about a 4-week cycle. In some embodiments, the flat dose is administered to the subject on days 1, 8, and 15 of a 4-week cycle. In some embodiments, the flat dose is administered to the subject on days 1, 8, and 15 of a 4-week cycle and the antibody-drug conjugate is brentuximab vedotin.

In one embodiment of the methods or uses or product for uses provided herein, an anti-CD30 antibody-drug conjugate or antigen-binding fragment thereof as described herein is administered to the subject at a flat dose ranging from 50 mg to 200 mg such as at a flat dose of 50 mg or a flat dose of 60 mg or a flat dose of 70 mg or a flat dose of 80 mg or a flat dose of 90 mg or a flat dose of 100 mg or a flat dose of 110 mg or a flat dose of 120 mg or a flat dose of 130 mg or a flat dose of 140 mg or a flat dose of 150 mg or a flat dose of 160 mg or a flat dose of 170 mg or a flat dose of 180 mg or a flat dose of 190 mg or a flat dose of 200 mg. In some embodiments, the flat dose is administered to the subject once about every 1 to 4 weeks. In certain embodiments, the flat dose is administered to the subject once about every 1 week, once about every 2 weeks, once about every 3 weeks or once about every 4 weeks. In some embodiments, the flat dose is administered to the subject once about every 3 weeks (e.g., f 3 days). In some embodiments, the flat dose is administered to the subject once every 3 weeks. In some embodiments, the flat dose is administered to the subject once every 3 weeks and the antibody-drug conjugate is brentuximab vedotin. In some embodiments, the flat does is administered to the subject once about every week (e.g., f 1 day). In some embodiments, the flat does is administered to the subject once every week. In some embodiments, the flat dose is administered to the subject once about every 1 week for 3 consecutive weeks followed by about a 1 week rest period without any administration of the anti-CD30 antibody-drug conjugate or antigen-binding fragment thereof so that each cycle time is about 28 days including the resting period. In some embodiments, the flat dose is administered to the subject once every 1 week for 3 consecutive weeks followed by a 1 week rest period without any administration of the anti-CD30 antibody-drug conjugate or antigen-binding fragment thereof so that each cycle time is 28 days including the resting period. In some embodiments, the flat dose is administered to the subject on about days 1, 8, and 15 of about a 4-week cycle. In some embodiments, the flat dose is administered to the subject on days 1, 8, and 15 of a 4-week cycle. In some embodiments, the flat dose is administered to the subject on days 1, 8, and 15 of a 4-week cycle and the antibody-drug conjugate is brentuximab vedotin.

In some embodiments, a method of treatment or use described herein further comprises the administration of one or more additional therapeutic agents. In some embodiments, the one or more additional therapeutic agents are administered simultaneously with an anti-CD30 antibody-drug conjugate or antigen-binding fragment thereof as described herein, such as brentuximab vedotin. In some embodiments, the one or more additional therapeutic agents and an anti-CD30 antibody-drug conjugate or antigen-binding fragment thereof as described herein, such as brentuximab vedotin, are administered sequentially.

D. Treatment Outcome

In one aspect, a method of treating an HIV infection with an anti-CD30 antibody-drug conjugate or antigen-binding fragment thereof as described herein results in an improvement in one or more therapeutic effects in the subject after administration of the antibody-drug conjugate relative to a baseline. In one aspect, a method of increasing CD4+ T-cell lymphocyte count in a subject infected with HIV with an anti-CD30 antibody-drug conjugate or antigen-binding fragment thereof as described herein results in an improvement in one or more therapeutic effects in the subject after administration of the antibody-drug conjugate relative to a baseline.

In one embodiment of the methods or uses or product for uses provided herein, the effectiveness of treatment with an anti-CD30 antibody-drug conjugate or antigen-binding fragment thereof described herein is assessed by measuring the HIV viral load in the subject. In some embodiments, the HIV viral load in the subject does not increase relative to the viral load prior to administration of an anti-CD30 antibody-drug conjugate or antigen-binding fragment thereof described herein. In some embodiments, the HIV viral load in the subject decreases relative to the viral load prior to administration of an anti-CD30 antibody-drug conjugate or antigen-binding fragment thereof described herein. In some embodiments, HIV viral load is assessed by measuring CD4⁺ T-cell-associated HIV DNA. In some embodiments, HIV viral load is assessed by measuring CD4⁺ T-cell-associated HIV RNA. In some embodiments, the subject exhibits a viral load of less than or equal to 50 copies of HIV virus particles per mL of blood plasma (<50 c/mL) after at least 24 weeks, at least 48 weeks, or at least 96 weeks after administration of the antibody-drug conjugate. In some embodiments, the subject exhibits a viral load of less than or equal to 50 copies of HIV virus particles per mL of blood plasma (<50 c/mL) after at least 24 weeks after administration of the antibody-drug conjugate. In some embodiments, the subject exhibits a viral load of less than or equal to 50 copies of HIV virus particles per mL of blood plasma (<50 c/mL) after at least 48 weeks after administration of the antibody-drug conjugate. In some embodiments, the subject exhibits a viral load of less than or equal to 50 copies of HIV virus particles per mL of blood plasma (<50 c/mL) after at least 96 weeks after administration of the antibody-drug conjugate. In some embodiments, administering an anti-CD30 antibody-drug conjugate or antigen-binding fragment thereof described herein results in elimination of the HIV infection in the subject.

In one embodiment of the methods or uses or product for uses provided herein, administering an anti-CD30 antibody-drug conjugate or antigen-binding fragment thereof described herein results in a decrease in the number of Treg cells relative to the number prior to the administration of the antibody-drug conjugate. In some embodiments, the Treg cells are CD4⁺. In some embodiments, the Treg cells are CD30⁺. In some embodiments, the Treg cells are CD4⁺ and CD30⁺. In one embodiment, the number of Treg cells decreases by at least about 5%, at least about 10%, at least about 15%, at least about 20%, at least about 25%, at least about 30%, at least about 40%, at least about 50%, at least about 60%, at least about 70%, at least about 80%, or at least about 90%.

In one embodiment of the methods or uses or product for uses provided herein, administering an anti-CD30 antibody-drug conjugate or antigen-binding fragment thereof described herein results in a decrease in the number of memory T cells relative to the number prior to the administration of the antibody-drug conjugate. In some embodiments, the memory T cells are CD4⁺. In some embodiments, the memory T cells are CD30⁺. In some embodiments, the memory T cells are CD4⁺ and CD30⁺. In one embodiment, the number of memory T cells decreases by at least about 5%, at least about 10%, at least about 15%, at least about 20%, at least about 25%, at least about 30%, at least about 40%, at least about 50%, at least about 60%, at least about 70%, at least about 80%, or at least about 90%.

In one embodiment of the methods or uses or product for uses provided herein, administering an anti-CD30 antibody-drug conjugate or antigen-binding fragment thereof described herein results in an increase in the number of memory T cells relative to the number prior to the administration of the antibody-drug conjugate. In some embodiments, the memory T cells are CD4⁺. In some embodiments, the memory T cells are CD30⁺. In some embodiments, the memory T cells are CD4⁺ and CD30⁺. In one embodiment, the number of memory T cells increases by at least about 5%, at least about 10%, at least about 15%, at least about 20%, at least about 25%, at least about 30%, at least about 40%, at least about 50%, at least about 60%, at least about 70%, at least about 80%, at least about 90%, at least about 100%, at least about 150%, at least about 200%, at least about 300%, or at least about 400%.

In one embodiment of the methods or uses or product for uses provided herein, administering an anti-CD30 antibody-drug conjugate or antigen-binding fragment thereof described herein results in an increase in the number of CD4⁺ T cells relative to the number prior to the administration of the antibody-drug conjugate. In one embodiment, the number of CD4⁺ T cells increases by at least about 5%, at least about 10%, at least about 15%, at least about 20%, at least about 25%, at least about 30%, at least about 40%, at least about 50%, at least about 60%, at least about 70%, at least about 80%, at least about 90%, at least about 100%, at least about 150%, at least about 20%, at least about 25%, at least about 30%, at least about 35%, or at least about 4⁰⁰%. In one embodiment, administering an anti-CD30 antibody-drug conjugate or antigen-binding fragment thereof described herein results in an increase in the number of CD4⁺ T cells relative to the number prior to the administration of the antibody-drug conjugate of at least about 10%. In one embodiment, administering an anti-CD30 antibody-drug conjugate or antigen-binding fragment thereof described herein results in an increase in the number of CD4⁺ T cells relative to the number prior to the administration of the antibody-drug conjugate of at least about 25%. In one embodiment, administering an anti-CD30 antibody-drug conjugate or antigen-binding fragment thereof described herein results in an increase in the number of CD4⁺ T cells relative to the number prior to the administration of the antibody-drug conjugate of at least about 50%. In one embodiment, administering an anti-CD30 antibody-drug conjugate or antigen-binding fragment thereof described herein results in an increase in the number of CD4⁺ T cells relative to the number prior to the administration of the antibody-drug conjugate of at least about 75%. In one embodiment, administering an anti-CD30 antibody-drug conjugate or antigen-binding fragment thereof described herein results in an increase in the number of CD4⁺ T cells relative to the number prior to the administration of the antibody-drug conjugate of at least about 100%. In one embodiment, administering an anti-CD30 antibody-drug conjugate or antigen-binding fragment thereof described herein results in an increase in the number of CD4⁺ T cells relative to the number prior to the administration of the antibody-drug conjugate of at least about 150%. In one embodiment, administering an anti-CD30 antibody-drug conjugate or antigen-binding fragment thereof described herein results in an increase in the number of CD4⁺ T cells relative to the number prior to the administration of the antibody-drug conjugate of at least about 200%. In one embodiment, administering an anti-CD30 antibody-drug conjugate or antigen-binding fragment thereof described herein results in an increase in the number of CD4⁺ T cells relative to the number prior to the administration of the antibody-drug conjugate of at least about 250%. In one embodiment, administering an anti-CD30 antibody-drug conjugate or antigen-binding fragment thereof described herein results in an increase in the number of CD4⁺ T cells relative to the number prior to the administration of the antibody-drug conjugate of at least about 300%. In one embodiment, administering an anti-CD30 antibody-drug conjugate or antigen-binding fragment thereof described herein results in an increase in the number of CD4⁺ T cells relative to the number prior to the administration of the antibody-drug conjugate of at least about 350%. In one embodiment, administering an anti-CD30 antibody-drug conjugate or antigen-binding fragment thereof described herein results in an increase in the number of CD4⁺ T cells relative to the number prior to the administration of the antibody-drug conjugate of at least about 400%.

In one embodiment of the methods or uses or product for uses provided herein, administering an anti-CD30 antibody-drug conjugate or antigen-binding fragment thereof described herein results in an increase in the number of CD4⁺ T cells relative to the number prior to the administration of the antibody-drug conjugate. In one embodiment, administering an anti-CD30 antibody-drug conjugate or antigen-binding fragment thereof described herein results in an increase in the CD4⁺ T-cell lymphocyte count in the subject of at least 25 cells/μL relative to the CD4⁺ T-cell lymphocyte count prior to administration. In one embodiment, administering an anti-CD30 antibody-drug conjugate or antigen-binding fragment thereof described herein results in an increase in the CD4⁺ T-cell lymphocyte count in the subject of at least 50 cells/μL relative to the CD4⁺ T-cell lymphocyte count prior to administration. In one embodiment, administering an anti-CD30 antibody-drug conjugate or antigen-binding fragment thereof described herein results in an increase in the CD4⁺ T-cell lymphocyte count in the subject of at least 75 cells/μL relative to the CD4⁺ T-cell lymphocyte count prior to administration. In one embodiment, administering an anti-CD30 antibody-drug conjugate or antigen-binding fragment thereof described herein results in an increase in the CD4⁺ T-cell lymphocyte count in the subject of at least 100 cells/μL relative to the CD4⁺ T-cell lymphocyte count prior to administration. In one embodiment, administering an anti-CD30 antibody-drug conjugate or antigen-binding fragment thereof described herein results in an increase in the CD4⁺ T-cell lymphocyte count in the subject of at least 150 cells/μL relative to the CD4⁺ T-cell lymphocyte count prior to administration. In one embodiment, administering an anti-CD30 antibody-drug conjugate or antigen-binding fragment thereof described herein results in an increase in the CD4⁺ T-cell lymphocyte count in the subject of at least 200 cells/μL relative to the CD4⁺ T-cell lymphocyte count prior to administration. In one embodiment, administering an anti-CD30 antibody-drug conjugate or antigen-binding fragment thereof described herein results in an increase in the CD4 T-cell lymphocyte count in the subject of at least 250 cells/μL relative to the CD4⁺ T-cell lymphocyte count prior to administration. In one embodiment, administering an anti-CD30 antibody-drug conjugate or antigen-binding fragment thereof described herein results in an increase in the CD4⁺ T-cell lymphocyte count in the subject of at least 300 cells/μL relative to the CD4⁺ T-cell lymphocyte count prior to administration. In one embodiment, administering an anti-CD30 antibody-drug conjugate or antigen-binding fragment thereof described herein results in an increase in the CD4⁺ T-cell lymphocyte count in the subject of at least 350 cells/μL relative to the CD4⁺ T-cell lymphocyte count prior to administration. In one embodiment, administering an anti-CD30 antibody-drug conjugate or antigen-binding fragment thereof described herein results in an increase in the CD4⁺ T-cell lymphocyte count in the subject of at least 400 cells/μL relative to the CD4⁺ T-cell lymphocyte count prior to administration. In one embodiment, administering an anti-CD30 antibody-drug conjugate or antigen-binding fragment thereof described herein results in an increase in the CD4⁺ T-cell lymphocyte count in the subject of at least 450 cells/μL relative to the CD4⁺ T-cell lymphocyte count prior to administration. In one embodiment, administering an anti-CD30 antibody-drug conjugate or antigen-binding fragment thereof described herein results in an increase in the CD4⁺ T-cell lymphocyte count in the subject of at least 500 cells/μL relative to the CD4⁺ T-cell lymphocyte count prior to administration. In one embodiment, administering an anti-CD30 antibody-drug conjugate or antigen-binding fragment thereof described herein results in an increase in the CD4⁺ T-cell lymphocyte count in the subject of at least 550 cells/μL relative to the CD4⁺ T-cell lymphocyte count prior to administration. In one embodiment, administering an anti-CD30 antibody-drug conjugate or antigen-binding fragment thereof described herein results in an increase in the CD4⁺ T-cell lymphocyte count in the subject of at least 600 cells/μL relative to the CD4⁺ T-cell lymphocyte count prior to administration. In one embodiment, administering an anti-CD30 antibody-drug conjugate or antigen-binding fragment thereof described herein results in an increase in the CD4 T-cell lymphocyte count in the subject of at least 650 cells/p. L relative to the CD4C T-cell lymphocyte count prior to administration. In one embodiment, administering an anti-CD30 antibody-drug conjugate or antigen-binding fragment thereof described herein results in an increase in the CD4⁺ T-cell lymphocyte count in the subject of at least 700 cells/μL relative to the CD4⁺ T-cell lymphocyte count prior to administration. In one embodiment, administering an anti-CD30 antibody-drug conjugate or antigen-binding fragment thereof described herein results in an increase in the CD4⁺ T-cell lymphocyte count in the subject of at least 750 cells/μL relative to the CD4⁺ T-cell lymphocyte count prior to administration. In one embodiment, administering an anti-CD30 antibody-drug conjugate or antigen-binding fragment thereof described herein results in an increase in the CD4⁺ T-cell lymphocyte count in the subject of at least 800 cells/μL relative to the CD4⁺ T-cell lymphocyte count prior to administration. In one embodiment, administering an anti-CD30 antibody-drug conjugate or antigen-binding fragment thereof described herein results in an increase in the CD4⁺ T-cell lymphocyte count in the subject to above 200 cells/μL. In one embodiment, administering an anti-CD30 antibody-drug conjugate or antigen-binding fragment thereof described herein results in an increase in the CD4⁺ T-cell lymphocyte count in the subject to above 250 cells/μL. In one embodiment, administering an anti-CD30 antibody-drug conjugate or antigen-binding fragment thereof described herein results in an increase in the CD4⁺ T-cell lymphocyte count in the subject to above 300 cells/μL. In one embodiment, administering an anti-CD30 antibody-drug conjugate or antigen-binding fragment thereof described herein results in an increase in the CD4⁺ T-cell lymphocyte count in the subject to above 350 cells/μL. In one embodiment, administering an anti-CD30 antibody-drug conjugate or antigen-binding fragment thereof described herein results in an increase in the CD4⁺ T-cell lymphocyte count in the subject to above 400 cells/μL. In one embodiment, administering an anti-CD30 antibody-drug conjugate or antigen-binding fragment thereof described herein results in an increase in the CD4⁺ T-cell lymphocyte count in the subject to above 450 cells/μL. In one embodiment, administering an anti-CD30 antibody-drug conjugate or antigen-binding fragment thereof described herein results in an increase in the CD4⁺ T-cell lymphocyte count in the subject to above 500 cells/μL. In one embodiment, administering an anti-CD30 antibody-drug conjugate or antigen-binding fragment thereof described herein results in an increase in the CD4⁺ T-cell lymphocyte count in the subject to above 550 cells/μL. In one embodiment, administering an anti-CD30 antibody-drug conjugate or antigen-binding fragment thereof described herein results in an increase in the CD4⁺ T-cell lymphocyte count in the subject to above 600 cells/μL. In one embodiment, administering an anti-CD30 antibody-drug conjugate or antigen-binding fragment thereof described herein results in an increase in the CD4 T-cell lymphocyte count in the subject to above 650 cells/μL. In one embodiment, administering an anti-CD30 antibody-drug conjugate or antigen-binding fragment thereof described herein results in an increase in the CD4 T-cell lymphocyte count in the subject to above 700 cells/μL. In one embodiment, administering an anti-CD30 antibody-drug conjugate or antigen-binding fragment thereof described herein results in an increase in the CD4⁺ T-cell lymphocyte count in the subject to above 750 cells/μL. In one embodiment, administering an anti-CD30 antibody-drug conjugate or antigen-binding fragment thereof described herein results in an increase in the CD4⁺ T-cell lymphocyte count in the subject to above 800 cells/μL. In one embodiment, administering an anti-CD30 antibody-drug conjugate or antigen-binding fragment thereof described herein results in an increase in the CD4⁺ T-cell lymphocyte count in the subject to above 850 cells/μL. In one embodiment, administering an anti-CD30 antibody-drug conjugate or antigen-binding fragment thereof described herein results in an increase in the CD4⁺ T-cell lymphocyte count in the subject to above 900 cells/μL. In one embodiment, administering an anti-CD30 antibody-drug conjugate or antigen-binding fragment thereof described herein results in an increase in the CD4 T-cell lymphocyte count in the subject to above 950 cells/μL. In one embodiment, administering an anti-CD30 antibody-drug conjugate or antigen-binding fragment thereof described herein results in an increase in the CD4⁺ T-cell lymphocyte count in the subject to above 1000 cells/μL.

In one embodiment of the methods or uses or product for uses provided herein, administering an anti-CD30 antibody-drug conjugate or antigen-binding fragment thereof described herein results in an increase in the number of CD8⁺ T cells relative to the number prior to the administration of the antibody-drug conjugate. In one embodiment, the number of CD8⁺ T cells increases by at least about 5%, at least about 10%, at least about 15%, at least about 20%, at least about 25%, at least about 30%, at least about 40%, at least about 50%, at least about 60%, at least about 70%, at least about 80%, at least about 90%, at least about 100%, at least about 150%, at least about 200%, at least about 250%, at least about 300%, at least about 350%, or at least about 400%. In one embodiment, administering an anti-CD30 antibody-drug conjugate or antigen-binding fragment thereof described herein results in an increase in the number of CD8⁺ T cells relative to the number prior to the administration of the antibody-drug conjugate of at least about 10%. In one embodiment, administering an anti-CD30 antibody-drug conjugate or antigen-binding fragment thereof described herein results in an increase in the number of CD8⁺ T cells relative to the number prior to the administration of the antibody-drug conjugate of at least about 25%. In one embodiment, administering an anti-CD30 antibody-drug conjugate or antigen-binding fragment thereof described herein results in an increase in the number of CD8⁺ T cells relative to the number prior to the administration of the antibody-drug conjugate of at least about 50%. In one embodiment, administering an anti-CD30 antibody-drug conjugate or antigen-binding fragment thereof described herein results in an increase in the number of CD8⁺ T cells relative to the number prior to the administration of the antibody-drug conjugate of at least about 75%. In one embodiment, administering an anti-CD30 antibody-drug conjugate or antigen-binding fragment thereof described herein results in an increase in the number of CD8⁺ T cells relative to the number prior to the administration of the antibody-drug conjugate of at least about 100%. In one embodiment, administering an anti-CD30 antibody-drug conjugate or antigen-binding fragment thereof described herein results in an increase in the number of CD8⁺ T cells relative to the number prior to the administration of the antibody-drug conjugate of at least about 150%. In one embodiment, administering an anti-CD30 antibody-drug conjugate or antigen-binding fragment thereof described herein results in an increase in the number of CD8⁺ T cells relative to the number prior to the administration of the antibody-drug conjugate of at least about 200%. In one embodiment, administering an anti-CD30 antibody-drug conjugate or antigen-binding fragment thereof described herein results in an increase in the number of CD8⁺ T cells relative to the number prior to the administration of the antibody-drug conjugate of at least about 250%. In one embodiment, administering an anti-CD30 antibody-drug conjugate or antigen-binding fragment thereof described herein results in an increase in the number of CD8⁺ T cells relative to the number prior to the administration of the antibody-drug conjugate of at least about 300%. In one embodiment, administering an anti-CD30 antibody-drug conjugate or antigen-binding fragment thereof described herein results in an increase in the number of CD8⁺ T cells relative to the number prior to the administration of the antibody-drug conjugate of at least about 350%. In one embodiment, administering an anti-CD30 antibody-drug conjugate or antigen-binding fragment thereof described herein results in an increase in the number of CD8⁺ T cells relative to the number prior to the administration of the antibody-drug conjugate of at least about 400%.

In one embodiment of the methods or uses or product for uses provided herein, administering an anti-CD30 antibody-drug conjugate or antigen-binding fragment thereof described herein results in a decrease in the number of CD8⁺ T-cells relative to the number prior to the administration of the antibody-drug conjugate. In one embodiment, the number of CD8⁺ T-cells decreases by at least about 5%, at least about 10%, at least about 15%, at least about 20%, at least about 25%, at least about 30%, at least about 40%, at least about 50%, at least about 60%, at least about 70%, at least about 80%, or at least about 90%.

In one embodiment of the methods or uses or product for uses provided herein, administering an anti-CD30 antibody-drug conjugate or antigen-binding fragment thereof described herein results in an increase in the CD4⁺:CD8⁺ T-cell lymphocyte ratio relative to the ratio prior to the administration of the antibody-drug conjugate. In some embodiments, the ratio is increased by at least 1.25:1, at least about 1.5:1, at least about 1.75:1, at least about 2:1, at least about 2.25:1, at least about 2.5:1, at least about 3:1, at least about 3.5:1, at least about 4:1, at least about 4.5:1, or at least about 5:1.

In one embodiment of the methods or uses or product for uses provided herein, administering an anti-CD30 antibody-drug conjugate or antigen-binding fragment thereof described herein results in an increase in the CD8⁺:CD4⁺ T-cell lymphocyte ratio relative to the ratio prior to the administration of the antibody-drug conjugate. In some embodiments, the ratio is increased by at least 1.25:1, at least about 1.5:1, at least about 1.75:1, at least about 2:1, at least about 2.25:1, at least about 2.5:1, at least about 3:1, at least about 3.5:1, at least about 4:1, at least about 4.5:1, or at least about 5:1.

### V. Compositions

In some aspects, provided herein are compositions (e.g., pharmaceutical compositions) comprising any of the anti-CD30 antibody-drug conjugates described herein (e.g., an anti-CD30 antibody-drug conjugate that binds to human CD30). The anti-CD30 antibody-drug conjugates of the present disclosure can be constituted in a composition, e.g., a pharmaceutical composition containing an antibody-drug conjugate and a pharmaceutically acceptable carrier. As used herein, a “pharmaceutically acceptable carrier” includes any and all solvents, dispersion media, coatings, antibacterial and antifungal agents, isotonic and absorption delaying agents, and the like that are physiologically compatible. In some embodiments, the carrier for a composition containing an antibody-drug conjugate is suitable for intravenous, intramuscular, subcutaneous, parenteral, spinal, or epidermal administration (e.g., by injection or infusion). A pharmaceutical composition of the disclosure can include one or more pharmaceutically acceptable salts, anti-oxidants, aqueous and non-aqueous carriers, and/or adjuvants such as preservatives, wetting agents, emulsifying agents, and dispersing agents.

Therapeutic formulations are prepared for storage by mixing the active ingredient having the desired degree of purity with optional pharmaceutically acceptable carriers, excipients or stabilizers (Remington: The Science and Practice of Pharmacy, 20th Ed., Lippincott Williams & Wiklins, Pub., Gennaro Ed., Philadelphia, Pa. 2000). Acceptable carriers, excipients, or stabilizers are nontoxic to recipients at the dosages and concentrations employed, and include buffers, antioxidants including ascorbic acid, methionine, Vitamin E, sodium metabisulfite; preservatives, isotonicifiers, stabilizers, metal complexes (e.g., Zn-protein complexes); chelating agents such as EDTA and/or non-ionic surfactants.

Buffers can be used to control the pH in a range which optimizes the therapeutic effectiveness, especially if stability is pH dependent. Buffers can be present at concentrations ranging from about 50 mM to about 250 mM. Suitable buffering agents for use with the present invention include both organic and inorganic acids and salts thereof. For example, citrate, phosphate, succinate, tartrate, fumarate, gluconate, oxalate, lactate, acetate. Additionally, buffers may be comprised of histidine and trimethylamine salts such as Tris.

Preservatives can be added to prevent microbial growth, and are typically present in a range from about 0.2%-1.0% (w/v). Suitable preservatives for use with the present invention include octadecyldimethylbenzyl ammonium chloride; hexamethonium chloride; benzalkonium halides (e.g., chloride, bromide, iodide), benzethonium chloride; thimerosal, phenol, butyl or benzyl alcohol; alkyl parabens such as methyl or propyl paraben; catechol; resorcinol; cyclohexanol, 3-pentanol, and m-cresol.

Tonicity agents, sometimes known as “stabilizers” can be present to adjust or maintain the tonicity of liquid in a composition. When used with large, charged biomolecules such as proteins and antibodies, they are often termed “stabilizers” because they can interact with the charged groups of the amino acid side chains, thereby lessening the potential for inter and intra-molecular interactions. Tonicity agents can be present in any amount between about 0.1% to about 25% by weight or between about 1 to about 5% by weight, taking into account the relative amounts of the other ingredients. In some embodiments, tonicity agents include polyhydric sugar alcohols, trihydric or higher sugar alcohols, such as glycerin, erythritol, arabitol, xylitol, sorbitol and mannitol.

Additional excipients include agents which can serve as one or more of the following: (1) bulking agents, (2) solubility enhancers, (3) stabilizers and (4) and agents preventing denaturation or adherence to the container wall. Such excipients include: polyhydric sugar alcohols (enumerated above); amino acids such as alanine, glycine, glutamine, asparagine, histidine, arginine, lysine, ornithine, leucine, 2-phenylalanine, glutamic acid, threonine, etc.; organic sugars or sugar alcohols such as sucrose, lactose, lactitol, trehalose, stachyose, mannose, sorbose, xylose, ribose, ribitol, myoinisitose, myoinisitol, galactose, galactitol, glycerol, cyclitols (e.g., inositol), polyethylene glycol; sulfur containing reducing agents, such as urea, glutathione, thioctic acid, sodium thioglycolate, thioglycerol, a-monothioglycerol and sodium thio sulfate; low molecular weight proteins such as human serum albumin, bovine serum albumin, gelatin or other immunoglobulins; hydrophilic polymers such as polyvinylpyrrolidone; monosaccharides (e.g., xylose, mannose, fructose, glucose; disaccharides (e.g., lactose, maltose, sucrose); trisaccharides such as raffinose; and polysaccharides such as dextrin or dextran.

Non-ionic surfactants or detergents (also known as “wetting agents”) can be present to help solubilize the therapeutic agent (e.g., anti-CD30 antibody-drug conjugate) as well as to protect the therapeutic protein (e.g., anti-CD30 antibody) against agitation-induced aggregation, which also permits the formulation to be exposed to shear surface stress without causing denaturation of the active therapeutic protein. Non-ionic surfactants are present in a range of about 0.05 mg/ml to about 1.0 mg/ml or about 0.07 mg/ml to about 0.2 mg/ml. In some embodiments, non-ionic surfactants are present in a range of about 0.001% to about 0.1% w/v or about 0.01% to about 0.1% w/v or about 0.01% to about 0.025% w/v.

Suitable non-ionic surfactants include polysorbates (20, 40, 60, 65, 80, etc.), polyoxamers (184, 188, etc.), PLURONIC® polyols, TRITON®, polyoxyethylene sorbitan monoethers (TWEEN®-20, TWEEN®-80, etc.), lauromacrogol 400, polyoxyl 40 stearate, polyoxyethylene hydrogenated castor oil 10, 50 and 60, glycerol monostearate, sucrose fatty acid ester, methyl celluose and carboxymethyl cellulose. Anionic detergents that can be used include sodium lauryl sulfate, dioctyle sodium sulfosuccinate and dioctyl sodium sulfonate. Cationic detergents include benzalkonium chloride or benzethonium chloride.

In order for the formulations to be used for in vivo administration, they must be sterile. The formulation may be rendered sterile by filtration through sterile filtration membranes. The therapeutic compositions herein generally are placed into a container having a sterile access port, for example, an intravenous solution bag or vial having a stopper pierceable by a hypodermic injection needle.

The route of administration is in accordance with known and accepted methods, such as by single or multiple bolus or infusion over a long period of time in a suitable manner, e.g., injection or infusion by subcutaneous, intravenous, intraperitoneal, intramuscular, intraarterial, intralesional or intraarticular routes, topical administration, inhalation or by sustained release or extended-release means.

The formulation herein may also contain more than one active compound as necessary for the particular indication being treated, preferably those with complementary activities that do not adversely affect each other. Alternatively, or in addition, the composition may comprise a cytotoxic agent, cytokine or growth inhibitory agent. Such molecules are suitably present in combination in amounts that are effective for the purpose intended.

The invention provides compositions comprising a population of ant-CD30 antibody-drug conjugates or antigen-binding fragments thereof as described herein for use in a method of treating an HIV infection as described herein. In some aspects, provided herein are compositions comprising a population of antibody-drug conjugates, wherein the antibody-drug conjugates comprise a linker attached to MMAE, wherein the antibody-drug conjugate has the following structure:

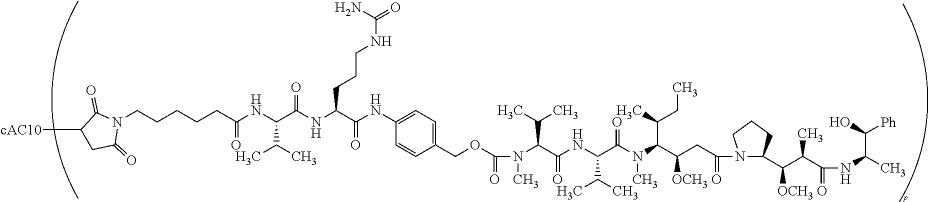

wherein p denotes a number from 1 to 8, e.g., 1, 2, 3, 4, 5, 6, 7 or 8, S represents a sulphydryl residue of the anti-CD30 antibody or antigen-binding fragment thereof, and cAC10 designates the anti-CD30 antibody or antigen-binding fragment thereof as described herein, such as brentuximab. In some embodiments, p denotes a number from 3 to 5. In some embodiments, the average value of p in the composition is about 4. In some embodiments, the population is a mixed population of antibody-drug conjugates in which p varies from 1 to 8 for each antibody-drug conjugate. In some embodiments, the population is a homogenous population of antibody-drug conjugates with each antibody-drug conjugate having the same value for p.

Dosage regimens are adjusted to provide the optimum desired response, e.g., a maximal therapeutic response and/or minimal adverse effects. In some embodiments, the anti-CD30 antibody-drug conjugate (e.g., brentuximab vedotin) is administered at a weight-based dose. For administration of an anti-CD30 antibody-drug conjugate (e.g., brentuximab vedotin), the dosage can range from about 0.01 mg/kg to about 20 mg/kg, about 0.05 mg/kg to about 20 mg/kg, about 0.1 mg/kg to about 20 mg/kg, about 0.1 mg/kg to about 15 mg/kg, about 0.1 mg/kg to about 10 mg/kg, about 0.1 mg/kg to about 5 mg/kg, about 0.1 mg/kg to about 4 mg/kg, about 0.1 mg/kg to about 3 mg/kg, about 0.1 mg/kg to about 2 mg/kg, about 0.1 mg/kg to about 1.5 mg/kg, about 0.1 mg/kg to about 1.3 mg/kg, about 1 mg/kg to about 10 mg/kg, about 1 mg/kg to about 10 mg/kg, about 1 mg/kg to about 8 mg/kg, about 1 mg/kg to about 5 mg/kg, about 1 mg/kg to about 3 mg/kg, about 1 mg/kg to about 2 mg/kg of the subject's body weight. For example, dosages can be about 0.05 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1.0 mg/kg, about 1.1 mg/kg, about 1.2 mg/kg, about 1.3 mg/kg, about 1.4 mg/kg, about 1.5 mg/kg, about 1.6 mg/kg, about 1.7 mg/kg, about 1.8 mg/kg, about 1.9 mg/kg, about 2.0 mg/kg, about 2.1 mg/kg, about 2.2 mg/kg, about 2.3 mg/kg, about 2.4 mg/kg, about 2.5 mg/kg, about 2.6 mg/kg, about 2.7 mg/kg, about 2.8 mg/kg, about 2.9 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, or about 20 mg/kg of the subject's body weight.

In some embodiments, the dosage of the anti-CD30 antibody-drug conjugate (e.g., brentuximab vedotin) is 0.1 mg/kg body weight. In other embodiments, the dosage of the anti-CD30 antibody-drug conjugate (e.g., brentuximab vedotin) is 0.2 mg/kg body weight. In other embodiments, the dosage of the anti-CD30 antibody-drug conjugate (e.g., brentuximab vedotin) is 0.3 mg/kg body weight. In other embodiments, the dosage of the anti-CD30 antibody-drug conjugate (e.g., brentuximab vedotin) is 0.4 mg/kg body weight. In other embodiments, the dosage of the anti-CD30 antibody-drug conjugate (e.g., brentuximab vedotin) is 0.5 mg/kg body weight. In other embodiments, the dosage of the anti-CD30 antibody-drug conjugate (e.g., brentuximab vedotin) is 0.6 mg/kg body weight. In other embodiments, the dosage of the anti-CD30 antibody-drug conjugate (e.g., brentuximab vedotin) is 0.7 mg/kg body weight. In other embodiments, the dosage of the anti-CD30 antibody-drug conjugate (e.g., brentuximab vedotin) is 0.8 mg/kg body weight. In other embodiments, the dosage of the anti-CD30 antibody-drug conjugate (e.g., brentuximab vedotin) is 0.9 mg/kg body weight. In other embodiments, the dosage of the anti-CD30 antibody-drug conjugate (e.g., brentuximab vedotin) is 1.0 mg/kg body weight. In other embodiments, the dosage of the anti-CD30 antibody-drug conjugate (e.g., brentuximab vedotin) is 1.1 mg/kg body weight. In other embodiments, the dosage of the anti-CD30 antibody-drug conjugate (e.g., brentuximab vedotin) is 1.2 mg/kg body weight. In other embodiments, the dosage of the anti-CD30 antibody-drug conjugate (e.g., brentuximab vedotin) is 1.3 mg/kg body weight. In other embodiments, the dosage of the anti-CD30 antibody-drug conjugate (e.g., brentuximab vedotin) is 1.4 mg/kg body weight. In other embodiments, the dosage of the anti-CD30 antibody-drug conjugate (e.g., brentuximab vedotin) is 1.5 mg/kg body weight. In other embodiments, the dosage of the anti-CD30 antibody-drug conjugate (e.g., brentuximab vedotin) is 1.6 mg/kg body weight. In other embodiments, the dosage of the anti-CD30 antibody-drug conjugate (e.g., brentuximab vedotin) is 1.7 mg/kg body weight. In other embodiments, the dosage of the anti-CD30 antibody-drug conjugate (e.g., brentuximab vedotin) is 1.8 mg/kg body weight. In other embodiments, the dosage of the anti-CD30 antibody-drug conjugate (e.g., brentuximab vedotin) is 1.9 mg/kg body weight. In other embodiments, the dosage of the anti-CD30 antibody-drug conjugate (e.g., brentuximab vedotin) is 2.0 mg/kg body weight. In other embodiments, the dosage of the anti-CD30 antibody-drug conjugate (e.g., brentuximab vedotin) is 2.1 mg/kg body weight. In other embodiments, the dosage of the anti-CD30 antibody-drug conjugate (e.g., brentuximab vedotin) is 2.2 mg/kg body weight. In other embodiments, the dosage of the anti-CD30 antibody-drug conjugate (e.g., brentuximab vedotin) is 2.3 mg/kg body weight. In other embodiments, the dosage of the anti-CD30 antibody-drug conjugate (e.g., brentuximab vedotin) is 2.4 mg/kg body weight. In other embodiments, the dosage of the anti-CD30 antibody-drug conjugate (e.g., brentuximab vedotin) is 2.5 mg/kg body weight. In other embodiments, the dosage of the anti-CD30 antibody-drug conjugate (e.g., brentuximab vedotin) is about 5 mg/kg body weight. In other embodiments, the dosage of the anti-CD30 antibody-drug conjugate (e.g., brentuximab vedotin) is about 10 mg/kg body weight.

In certain embodiments, an anti-CD30 antibody-drug conjugate (e.g., brentuximab vedotin) is administered at a flat dose. In some embodiments, the flat dose of the anti-CD30 antibody is a dose (e.g., flat dose) of at least about 1 mg to about 1500 mg, at least about 10 mg to about 1000 mg, such as, at least about 50 mg to about 800 mg, at least about 100 mg to about 600 mg, at least about 100 mg to about 400 mg or at least about 100 mg to about 200 mg, such as at least about 1 mg, at least about 3 mg, at least about 5 mg, at least about 8 mg, at least about 10 mg, at least about 20 mg, at least about 30 mg, at least about 40 mg, at least about 50 mg, at least about 60 mg, at least about 70 mg, at least about 80 mg, at least about 90 mg, at least about 100 mg, at least about 110 mg, at least about 120 mg, at least about 130 mg, at least about 140 mg, at least about 150 mg, at least about 160 mg, at least about 170 mg, at least about 180 mg, at least about 190 mg, at least about 200 mg, at least about 220 mg, at least about 240 mg, at least about 260 mg, at least about 280 mg, at least about 300 mg, at least about 320 mg, at least about 340 mg, at least about 360 mg, at least about 380 mg, at least about 400 mg, at least about 420 mg, at least about 440 mg, at least about 460 mg, at least about 480 mg, at least about 500 mg, at least about 600 mg, at least about 700 mg, at least about 800 mg, at least about 900 mg, at least about 1000 mg, at least about 1100 mg, at least about 1200 mg, at least about 1300 mg, at least about 1400 mg, or at least about 1500 mg.

In certain embodiments, an anti-CD30 antibody-drug conjugate described herein (e.g., brentuximab vedotin) is administered at a flat dose. In some embodiments, the flat dose of the anti-CD30 antibody-drug conjugate is a dose (e.g., flat dose) of about 1 mg to about 1500 mg, about 10 mg to about 1000 mg, such as, about 50 mg to about 800 mg, about 100 mg to about 600 mg, about 100 mg to about 400 mg or about 100 to about 200 mg, such as about 1 mg, about 3 mg, about 5 mg, about 8 mg, about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 220 mg, about 240 mg, about 260 mg, about 280 mg, about 300 mg, about 320 mg, about 340 mg, about 360 mg, about 380 mg, about 400 mg, about 420 mg, about 440 mg, about 460 mg, about 480 mg, about 500 mg, about 600 mg, about 700 mg, about 800 mg, about 900 mg, about 1000 mg, about 1100 mg, about 1200 mg, about 1300 mg, about 1400 mg, or about 1500 mg.

An exemplary dosage regimen entails administration once per week, once about every 2 weeks, once about every 3 weeks, once about every 4 weeks, once about a month, once about every 3-6 months or longer. In certain embodiments, the anti-CD30 antibody-drug conjugate (e.g., brentuximab vedotin) is administered once about every 3 weeks. In certain embodiments, the anti-CD30 antibody-drug conjugate (e.g., brentuximab vedotin) is administered once about every 2 weeks.

In some embodiments, a subtherapeutic dose of an anti-CD30 antibody-drug conjugate (e.g., brentuximab vedotin) is used in the methods herein. The subtherapeutic dosages of an anti-CD30 antibody-drug conjugate (e.g., brentuximab vedotin) used in the methods herein are higher than 0.001 mg/kg and lower than 10 mg/kg. In some embodiments, the subtherapeutic dose is about 0.001 mg/kg-about 10 mg/kg, about 0.01 mg/kg-about 10 mg/kg, about 0.01 mg/kg-about 1 mg/kg, about 0.1 mg/kg-about 1 mg/kg, or about 0.001 mg/kg-about 0.1 mg/kg body weight. In some embodiments, the subtherapeutic dose is at least about 0.001 mg/kg, at least about 0.005 mg/kg, at least about 0.01 mg/kg, at least about 0.05 mg/kg, at least about 0.1 mg/kg, at least about 0.2 mg/kg, at least about 0.3 mg/kg, at least about 0.4 mg/kg, at least about 0.5 mg/kg, at least about 0.6 mg/kg, at least about 0.7 mg/kg, at least about 0.8 mg/kg, at least about 0.9 mg/kg, at least about 1 mg/kg, at least about 1.1 mg/kg, at least about 1.2 mg/kg, at least about 1.3 mg/kg, at least about 1.4 mg/kg, at least about 1.5 mg/kg, at least about 1.6 mg/kg, or at least about 1.7 mg/kg body weight.

In some embodiments, treatment is continued as long as clinical benefit is observed or until unacceptable toxicity or disease progression occurs.

Dosage and frequency vary depending on the half-life of the therapeutic agent (e.g., anti-CD30 antibody-drug conjugate) in the subject. In general, human antibodies show the longest half-life, followed by humanized antibodies, chimeric antibodies, and nonhuman antibodies. The dosage and frequency of administration can vary depending on whether the treatment is prophylactic or therapeutic. In prophylactic applications, a relatively low dosage is typically administered at relatively infrequent intervals over a long period of time. Some patients continue to receive treatment for the rest of their lives. In therapeutic applications, a relatively high dosage at relatively short intervals is sometimes required until progression of the disease is reduced or terminated, and until the patient shows partial or complete amelioration of symptoms of disease. Thereafter, the patient can be administered a prophylactic regime.

Actual dosage levels of the active ingredients in the pharmaceutical compositions of the present disclosure can be varied so as to obtain an amount of the active ingredient which is effective to achieve the desired therapeutic response for a particular patient, composition, and mode of administration, without being unduly toxic to the patient. The selected dosage level will depend upon a variety of pharmacokinetic factors including the activity of the particular compositions of the present disclosure employed, the route of administration, the time of administration, the rate of excretion of the particular compound being employed, the duration of the treatment, other drugs, compounds and/or materials used in combination with the particular compositions employed, the age, sex, weight, condition, general health, and prior medical history of the patient being treated, and like factors well known in the medical arts. A composition of the present disclosure can be administered via one or more routes of administration using one or more of a variety of methods well known in the art. As will be appreciated by the skilled artisan, the route and/or mode of administration will vary depending upon the desired results.

In some embodiments, a composition comprising an anti-CD30 antibody-drug conjugate as described herein is coadministered with one or additional therapeutic agents. In some embodiments the coadministration is simultaneous or sequential. In some embodiments, the anti-CD30 antibody-drug conjugate as described herein is administered simultaneously with the one or more additional therapeutic agents. In some embodiments, simultaneous means that the anti-CD30 antibody-drug conjugate described herein and the one or more therapeutic agents are administered to the subject less than about one hour apart, such as less than about 30 minutes apart, less than about 15 minutes apart, less than about 10 minutes apart or less than about 5 minutes apart. In some embodiments, simultaneous means that the anti-CD30 antibody-drug conjugate described herein and the one or more therapeutic agents are administered to the subject less than one hour apart, such as less than 30 minutes apart, less than 15 minutes apart, less than 10 minutes apart or less than 5 minutes apart. In some embodiments, the anti-CD30 antibody-drug conjugate described herein is administered sequentially with the one or more additional therapeutic agents. In some embodiments, sequential administration means that the anti-CD30 antibody-drug conjugate described herein and the one or more additional therapeutic agents are administered a least 1 hour apart, at least 2 hours apart, at least 3 hours apart, at least 4 hours apart, at least 5 hours apart, at least 6 hours apart, at least 7 hours apart, at least 8 hours apart, at least 9 hours apart, at least 10 hours apart, at least 11 hours apart, at least 12 hours apart, at least 13 hours apart, at least 14 hours apart, at least 15 hours apart, at least 16 hours apart, at least 17 hours apart, at least 18 hours apart, at least 19 hours apart, at least 20 hours apart, at least 21 hours apart, at least 22 hours apart, at least 23 hours apart, at least 24 hours apart, at least 2 days apart, at least 3 days apart, at least 4 days apart, at least 5 days apart, at least 5 days apart, at least 7 days apart, at least 2 weeks apart, at least 3 weeks apart or at least 4 weeks apart.

In some embodiments, a composition comprising an anti-CD30 antibody-drug conjugate as described herein is coadministered with one or more therapeutic agents to eliminate or reduce the severity of one or more adverse events. In some embodiments the coadministration is simultaneous or sequential. In some embodiments, the anti-CD30 antibody-drug conjugate described herein is administered simultaneously with the one or more therapeutic agents to eliminate or reduce the severity of one or more adverse events. In some embodiments, simultaneous means that the anti-CD30 antibody-drug conjugate described herein and the one or more therapeutic agents to eliminate or reduce the severity of one or more adverse events are administered to the subject less than about one hour apart, such as less than about 30 minutes apart, less than about 15 minutes apart, less than about 10 minutes apart or less than about 5 minutes apart. In some embodiments, simultaneous means that the anti-CD30 antibody-drug conjugate described herein and the one or more therapeutic agents to eliminate or reduce the severity of one or more adverse events are administered to the subject less than one hour apart, such as less than 30 minutes apart, less than 15 minutes apart, less than 10 minutes apart or less than 5 minutes apart. In some embodiments, the anti-CD30 antibody-drug conjugate described herein is administered sequentially with the one or more therapeutic agents to eliminate or reduce the severity of one or more adverse events. In some embodiments, sequential administration means that the anti-CD30 antibody-drug conjugate described herein and the one or more additional therapeutic agents are administered a least 1 hour apart, at least 2 hours apart, at least 3 hours apart, at least 4 hours apart, at least 5 hours apart, at least 6 hours apart, at least 7 hours apart, at least 8 hours apart, at least 9 hours apart, at least 10 hours apart, at least 11 hours apart, at least 12 hours apart, at least 13 hours apart, at least 14 hours apart, at least 15 hours apart, at least 16 hours apart, at least 17 hours apart, at least 18 hours apart, at least 19 hours apart, at least 20 hours apart, at least 21 hours apart, at least 22 hours apart, at least 23 hours apart, at least 24 hours apart, at least 2 days apart, at least 3 days apart, at least 4 days apart, at least 5 days apart, at least 5 days apart, at least 7 days apart, at least 2 weeks apart, at least 3 weeks apart or at least 4 weeks apart. In some embodiments, the anti-CD30 antibody-drug conjugate described herein is administered prior to the one or more therapeutic agents to eliminate or reduce the severity of one or more adverse events. In some embodiments, the one or more therapeutic agents to eliminate or reduce the severity of one or more adverse events is administered prior to the anti-CD30 antibody-drug conjugate described herein.

### VI. Articles of Manufacture or Kits

Also within the scope of the present disclosure provides an article of manufacture or kit which comprises a therapeutic agent described herein (e.g., an anti-CD30 antibody-drug conjugate). The article of manufacture or kit may further comprise instructions for use of the therapeutic agent (e.g., an anti-CD30 antibody-drug conjugate) in the methods of the invention. An article of manufacture or kit typically includes a label indicating the intended use of the contents of the article of manufacture or kit and instructions for use. The term label includes any writing, or recorded material supplied on or with the article of manufacture or kit. Thus, in certain embodiments, the article of manufacture or kit comprises instructions for the use of an anti-CD30 antibody-drug conjugate (e.g., brentuximab vedotin) in any of the methods disclosed herein.

In some embodiments, provided herein is an article of manufacture or kit for treating a subject afflicted with a HIV (e.g., having an HIV infection), the kit comprising: (a) a dosage ranging from about 0.1 mg to about 500 mg of an anti-CD30 antibody-drug conjugate; and (b) instructions for using the anti-CD30 antibody-drug conjugate in any of the methods disclosed herein. In certain embodiments for treating human patients, the article of manufacture or kit comprises an anti-human CD30 antibody-drug conjugate disclosed herein, e.g., brentuximab vedotin.

The article of manufacture or kit may further comprise a container. Suitable containers include, for example, bottles, vials (e.g., dual chamber vials), syringes (such as single or dual chamber syringes) and test tubes. The container may be formed from a variety of materials such as glass or plastic. The container holds the formulation.

The article of manufacture or kit may further comprise a label or a package insert, which is on or associated with the container, may indicate directions for reconstitution and/or use of the formulation. The label or package insert may further indicate that the formulation is useful or intended for subcutaneous, intravenous, or other modes of administration in an individual. The container holding the formulation may be a single-use vial or a multi-use vial, which allows for repeat administrations of the reconstituted formulation. The article of manufacture or kit may further comprise a second container comprising a suitable diluent. The article of manufacture or kit may further include other materials desirable from a commercial, therapeutic, and user standpoint, including other buffers, diluents, filters, needles, syringes, and package inserts with instructions for use.

In a specific embodiment, the present invention provides kits for a single dose-administration unit. Such kits comprise a container of an aqueous formulation of therapeutic antibody, including both single or multi-chambered pre-filled syringes. Exemplary pre-filled syringes are available from Vetter GmbH, Ravensburg, Germany.

In some embodiments, the anti-CD30 antibody-drug conjugate described herein is present in the container as a lyophilized powder. In some embodiments, the lyophilized powder is in a hermetically sealed container, such as a vial, an ampoule or sachette, indicating the quantity of the active agent. Where the pharmaceutical is administered by injection, an ampoule of sterile water for injection or saline can be, for example, provided, optionally as part of the kit, so that the ingredients can be mixed prior to administration. Such kits can further include, if desired, one or more of various conventional pharmaceutical components, such as, for example, containers with one or more pharmaceutically acceptable carriers, additional containers, etc., as will be readily apparent to those skilled in the art. Printed instructions, either as inserts or as labels, indicating quantities of the components to be administered, guidelines for administration, and/or guidelines for mixing the components can also be included in the kit.

The present invention also provides an anti-CD30 antibody-drug conjugate described herein that binds to CD30 (e.g., human CD30) in combination with one or more therapeutic agent (e.g., a second therapeutic agent) for use in any of the methods disclosed herein. In some embodiments, the article of manufacture or kit herein optionally further comprises a container comprising a second therapeutic medicament (e.g., a second therapeutic agent), wherein the anti-CD30 antibody-drug conjugate is a first medicament (e.g., a first therapeutic agent), and which article or kit further comprises instructions on the label or package insert for treating the individual with the second medicament, in an effective amount.

In another embodiment, provided herein is an article of manufacture or kit comprising the formulations described herein for administration in an auto-injector device. An auto-injector can be described as an injection device that upon activation, will deliver its contents without additional necessary action from the patient or administrator. They are particularly suited for self-medication of therapeutic formulations when the delivery rate must be constant and the time of delivery is greater than a few moments.

### VII. Exemplary Embodiments

Among the embodiments provided herein are:

1. A method of treating an HIV infection in a subject comprising administering to the subject an antibody-drug conjugate, wherein the antibody-drug conjugate comprises an anti-CD30 antibody or an antigen-binding portion thereof conjugated to a monomethyl auristatin.
2. The method of embodiment 1, wherein the HIV infection is an HIV-1 infection.
3. The method of embodiment 1 or 2, wherein the subject does not have a hematologic cancer at the time of administration of the antibody-drug conjugate.
4. The method of embodiment 1 or 2, wherein the subject has not had a hematologic cancer for at least 12 months prior to the administration of the antibody-drug conjugate.
5. The method of embodiment 1 or 2, wherein the subject has not had a hematologic cancer for at least 24 months prior to the administration of the antibody-drug conjugate.
6. The method of any one of embodiments 3-5, wherein the hematologic cancer is selected from the group consisting of classical Hodgkin Lymphoma, non-Hodgkin Lymphoma, cutaneous T-cell lymphoma (CTCL), and anaplastic large cell lymphoma (ALCL).
7. The method of embodiment 6, wherein the hematologic cancer is classical Hodgkin Lymphoma.
8. The method of embodiment 7, wherein the classical Hodgkin Lymphoma is a stage IIA with bulky disease, stage IIB, stage III or stage IV classical Hodgkin Lymphoma.
9. The method of embodiment 6, wherein the anaplastic large cell lymphoma (ALCL) is a systemic anaplastic large cell lymphoma (sALCL).
10. The method of embodiment 6, wherein the anaplastic large cell lymphoma (ALCL) is a primary cutaneous anaplastic large cell lymphoma (pcALCL).
11. The method of embodiment 6, wherein the cutaneous T-cell lymphoma (CTCL) is a mycosis fungoides (MF).
12. The method of embodiment 11, wherein the mycosis fungoides (MF) is a CD30-positive mycosis fungoides (MF).
13. The method of any one of embodiments 1-12, wherein the anti-CD30 antibody of the antibody-drug conjugate comprises a heavy chain variable region and a light chain variable region, wherein the heavy chain variable region comprises:

(i) a CDR-H1 comprising the amino acid sequence of SEQ ID NO: 1;

(ii) a CDR-H2 comprising the amino acid sequence of SEQ ID NO: 2; and

(iii) a CDR-H3 comprising the amino acid sequence of SEQ ID NO: 3; and wherein the light chain variable region comprises:

(i) a CDR-L1 comprising the amino acid sequence of SEQ ID NO: 4;

(ii) a CDR-L2 comprising the amino acid sequence of SEQ ID NO: 5; and

(iii) a CDR-L3 comprising the amino acid sequence of SEQ ID NO: 6.

14. The method of any one of embodiments 1-13, wherein the anti-CD30 antibody of the antibody-drug conjugate comprises a heavy chain variable region comprising an amino acid sequence at least 85% identical to the amino acid sequence of SEQ ID NO: 7 and a light chain variable region comprising an amino acid sequence at least 85% identical to the amino acid sequence of SEQ ID NO: 8.
15. The method of any one of embodiments 1-13, wherein the anti-CD30 antibody of the antibody-drug conjugate comprises a heavy chain variable region comprising an amino acid sequence at least 90% identical to the amino acid sequence of SEQ ID NO: 7 and a light chain variable region comprising an amino acid sequence at least 90% identical to the amino acid sequence of SEQ ID NO: 8.
16. The method of any one of embodiments 1-13, wherein the anti-CD30 antibody of the antibody-drug conjugate comprises a heavy chain variable region comprising an amino acid sequence at least 95% identical to the amino acid sequence of SEQ ID NO: 7 and a light chain variable region comprising an amino acid sequence at least 95% identical to the amino acid sequence of SEQ ID NO: 8.
17. The method of any one of embodiments 1-12, wherein the anti-CD30 antibody of the antibody-drug conjugate comprises a heavy chain variable region comprising the amino acid sequence of SEQ ID NO: 7 and a light chain variable region comprising the amino acid sequence of SEQ ID NO: 8.
18. The method of any one of embodiments 1-12, wherein the anti-CD30 antibody is AC10.
19. The method of any one of embodiments 1-12, wherein the anti-CD30 antibody is cAC10.
20. The method of any one of embodiments 1-19, wherein the antibody-drug conjugate further comprises a linker between the anti-CD30 antibody or antigen-binding portion thereof and the monomethyl auristatin.
21. The method of embodiment 20, wherein the linker is a cleavable peptide linker.
22. The method of embodiment 21, wherein the cleavable peptide linker has a formula: -MC-vc-PAB-.
23. The method of any one of embodiments 1-22, wherein the monomethyl auristatin is monomethyl auristatin E (MMAE).
24. The method of any one of embodiments 1-22, wherein the monomethyl auristatin is monomethyl auristatin F (MMAF).
25. The method of any one of embodiments 1-12, wherein the antibody-drug conjugate is brentuximab vedotin.
26. The method of any one of embodiments 1-25, wherein the antibody-drug conjugate is administered at a dose ranging from of about 0.1 mg/kg to about 1.3 mg/kg of the subject's body weight.
27. The method of embodiment 26, wherein the antibody-drug conjugate is administered at a dose ranging from about 0.3 mg/kg to about 0.9 mg/kg of the subject's body weight.
28. The method of embodiment 26, wherein the antibody-drug conjugate is administered at a dose of about 0.3 mg/kg of the subject's body weight.
29. The method of embodiment 26, wherein the antibody-drug conjugate is administered at a dose of about 0.6 mg/kg of the subject's body weight.
30. The method of embodiment 26, wherein the antibody-drug conjugate is administered at a dose of about 0.9 mg/kg of the subject's body weight.
31. The method of any one of embodiments 1-30, wherein the antibody-drug conjugate is administered once about every 3 weeks.
32. The method of any one of embodiments 1-30, wherein the antibody-drug conjugate is administered once every 3 weeks.
33. The method of embodiment 31 or 32, wherein the antibody-drug conjugate is administered for 6 3-week treatment cycles.
34. The method of any one of embodiments 1-33, wherein the antibody-drug conjugate is administered to the subject by intravenous infusion.
35. The method of embodiment 34, wherein the intravenous infusion is about a 30 minute infusion.
36. The method of any one of embodiments 1-35, wherein the subject has a CD4 lymphocyte count of <200 cells/mm³ prior to administration of the antibody-drug conjugate.
37. The method of any one of embodiments 1-36, wherein the subject has plasma HIV RNA ≥1000 copies/mL prior to administration of the antibody-drug conjugate.
38. The method of any one of embodiments 1-37, wherein the subject has had plasma HIV RNA ≥200 copies/mL in a 3-month period prior to administration of the antibody-drug conjugate.
39. The method of any one of embodiments 1-38, wherein the subject has a life expectancy of greater than 9 months prior to administration of the antibody-drug conjugate.
40. The method of any one of embodiments 1-39, wherein the subject has an absolute neutrophil count ≥750/mm³.
41. The method of any one of embodiments 1-40, wherein the subject is male and has hemoglobin ≥10.5 gm/dL.
42. The method of any one of embodiments 1-40, wherein the subject is female and has hemoglobin ≥9.5 gm/dL.
43. The method of any one of embodiments 1-42, wherein the subject has serum alanine transaminase (SGPT/ALT)<2.5×upper limit of normal (ULN).
44. The method of any one of embodiments 1-43, wherein the subject has serum aspartate transaminase (SGOT/AST)<2.5×ULN.
45. The method of any one of embodiments 1-44, wherein the subject has bilirubin (total)<2.5×ULN.
46. The method of any one of embodiments 1-44, wherein the subject has creatinine <1.5×ULN.
47. The method of any one of embodiments 1-46, wherein the subject has received antiretroviral therapy (ART) for at least 24 weeks prior to administration of the antibody-drug conjugate.
48. The method of embodiment 47, wherein the subject has received ART for at least 12 months prior to administration of the antibody-drug conjugate.
49. The method of embodiment 48, wherein the subject has received ART for at least 24 months prior to administration of the antibody-drug conjugate.
50. The method of any one of embodiments 1-49, wherein the antibody-drug conjugate is administered in combination with ART.
51. The method of any one of embodiments 47-50, wherein the ART is a nucleoside reverse transcriptase inhibitor, non-nucleoside reverse transcriptase inhibitor, protease inhibitor, fusion inhibitor, CCR5 antagonist, integrase inhibitor, post-attachment inhibitor, or pharmacokinetic enhancer.
52. The method of embodiment 51, wherein the ART comprises two or more of a nucleoside reverse transcriptase inhibitor, non-nucleoside reverse transcriptase inhibitor, protease inhibitor, fusion inhibitor, CCR5 antagonist, integrase inhibitor, post-attachment inhibitor, and pharmacokinetic enhancer.
53. The method of embodiment 51, wherein the ART comprises three or more of a nucleoside reverse transcriptase inhibitor, non-nucleoside reverse transcriptase inhibitor, protease inhibitor, fusion inhibitor, CCR5 antagonist, integrase inhibitor, post-attachment inhibitor, and pharmacokinetic enhancer.
54. The method of embodiment 51, wherein the ART comprises four or more of a nucleoside reverse transcriptase inhibitor, non-nucleoside reverse transcriptase inhibitor, protease inhibitor, fusion inhibitor, CCR5 antagonist, integrase inhibitor, post-attachment inhibitor, and pharmacokinetic enhancer.
55. The method of any one of embodiments 47-54, wherein the ART comprises one or more of abacavir, emtricitabine, lamivudine, tenofovir disoproxil fumarate, zidovudine, doravirine, efavirenz, etravirine, nevirapine, rilpivirine, atazanavir, darunavir, fosamprenavir, ritonavir, saquinavir, tipranavir, enfuvirtide, maraviroc, dolutegravir, raltegravir, ibalizumab, and cobicistat.
56. The method of any one of embodiments 1-55, wherein administering the antibody-drug conjugate results in a decrease in HIV viral load in the subject relative to the viral load prior to administration of the antibody-drug conjugate.
57. The method of embodiment 56, wherein HIV viral load is assessed by measuring CD4+ T-cell-associated HIV DNA.
58. The method of embodiment 56, wherein HIV viral load is assessed by measuring CD4+ T-cell-associated HIV RNA.
59. The method of any one of embodiments 1-58, wherein the subject exhibits a viral load of less than or equal to 50 copies of HIV virus particles per mL of blood plasma (<50 c/mL) after at least 24 weeks, at least 48 weeks, or at least 96 weeks after administration of the antibody-drug conjugate.
60. The method of any one of embodiments 1-59, wherein administering the antibody-drug conjugate results in elimination of the HIV infection in the subject.
61. The method of any one of embodiments 1-60, wherein administering the antibody-drug conjugate results in a decrease in the number of Treg cells relative to the number prior to the administration of the antibody-drug conjugate.
62. The method of embodiment 61, wherein the Treg cells are CD4+.
63. The method of embodiment 61 or 62, wherein the Treg cells are CD30+.
64. The method of any one of embodiments 1-63, wherein administering the antibody-drug conjugate results in a decrease in the number of memory T cells relative to the number prior to the administration of the antibody-drug conjugate.
65. The method of embodiment 61, wherein the memory T cells are CD4+.
66. The method of embodiment 61 or 62, wherein the memory T cells are CD30+.
67. The method of any one of embodiments 1-66, wherein administering the antibody-drug conjugate results in an increase in the number of CD4+ T cells relative to the number prior to the administration of the antibody-drug conjugate.
68. The method of any one of embodiments 1-67, wherein the subject is a human.
69. A kit comprising:

(a) a dosage ranging from about 0.1 mg to about 500 mg of an of an antibody-drug conjugate that binds to CD30, wherein the antibody-drug conjugate comprises an anti-CD30 antibody or an antigen-binding fragment thereof conjugated to a monomethyl auristatin or a functional analog thereof or a functional derivative thereof; and

(b) instructions for using the antibody drug conjugate according to the method of any one of embodiments 1-68.

70. Use of an antibody-drug conjugate that binds to CD30 for the manufacture of a medicament for use in the method of any one of embodiments 1-68.
71. An antibody-drug conjugate that binds to CD30 for use in the method of any one of embodiments 1-68.
72. A method of increasing CD4⁺ T-cell lymphocyte count in a subject infected with human immunodeficiency virus (HIV) comprising administering to the subject an antibody-drug conjugate, wherein the antibody-drug conjugate comprises an anti-CD30 antibody or an antigen-binding portion thereof conjugated to a monomethyl auristatin.
73. The method of embodiment 72, wherein the HIV infection is an HIV-1 infection.
74. The method of embodiment 72 or embodiment 73, wherein the subject has a CD4⁺ T-cell lymphocyte count of <200 cells/μL prior to administration of the antibody-drug conjugate.
75. The method of any one of embodiments 72-74, wherein the subject has a CD4⁺ T-cell lymphocyte count of ≥50 cells/μL prior to administration of the antibody-drug conjugate.
76. The method of any one of embodiments 72-75, wherein the subject has had a plasma HIV viral load ≤50 copies/mL for at least 6 months prior to administration of the antibody-drug conjugate.
77. The method of any one of embodiments 72-75, wherein the subject has had a plasma HIV viral load ≤50 copies/mL for at least 12 months prior to administration of the antibody-drug conjugate.
78. The method of any one of embodiments 72-75, wherein the subject has had a plasma HIV viral load <50 copies/mL for at least 24 months prior to administration of the antibody-drug conjugate.
79. The method of any one of embodiments 72-78, wherein the subject does not have a hematologic cancer at the time of administration of the antibody-drug conjugate.
80. The method of any one of embodiments 72-78, wherein the subject has not had a hematologic cancer for at least 12 months prior to the administration of the antibody-drug conjugate.
81. The method of any one of embodiments 72-78, wherein the subject has not had a hematologic cancer for at least 24 months prior to the administration of the antibody-drug conjugate.
82. The method of any one of embodiments 79-81, wherein the hematologic cancer is selected from the group consisting of classical Hodgkin Lymphoma, non-Hodgkin Lymphoma, cutaneous T-cell lymphoma (CTCL), and anaplastic large cell lymphoma (ALCL).
83. The method of embodiment 82, wherein the hematologic cancer is classical Hodgkin Lymphoma.
84. The method of embodiment 83, wherein the classical Hodgkin Lymphoma is a stage IIA with bulky disease, stage IIB, stage III or stage IV classical Hodgkin Lymphoma.
85. The method of embodiment 82, wherein the anaplastic large cell lymphoma (ALCL) is a systemic anaplastic large cell lymphoma (sALCL).
86. The method of embodiment 82, wherein the anaplastic large cell lymphoma (ALCL) is a primary cutaneous anaplastic large cell lymphoma (pcALCL).
87. The method of embodiment 82, wherein the cutaneous T-cell lymphoma (CTCL) is a mycosis fungoides (MF).
88. The method of embodiment 87, wherein the mycosis fungoides (MF) is a CD30-positive mycosis fungoides (MF).
89. The method of any one of embodiments 72-88, wherein the anti-CD30 antibody of the antibody-drug conjugate comprises a heavy chain variable region and a light chain variable region, wherein the heavy chain variable region comprises:

(i) a CDR-H1 comprising the amino acid sequence of SEQ ID NO: 1;

(ii) a CDR-H2 comprising the amino acid sequence of SEQ ID NO: 2; and

(iii) a CDR-H3 comprising the amino acid sequence of SEQ ID NO: 3; and

wherein the light chain variable region comprises:

(i) a CDR-L1 comprising the amino acid sequence of SEQ ID NO: 4:

(ii) a CDR-L2 comprising the amino acid sequence of SEQ ID NO: 5; and

(iii) a CDR-L3 comprising the amino acid sequence of SEQ ID NO: 6.

90. The method of any one of embodiments 71-89, wherein the anti-CD30 antibody of the antibody-drug conjugate comprises a heavy chain variable region comprising an amino acid sequence at least 85% identical to the amino acid sequence of SEQ ID NO: 7 and a light chain variable region comprising an amino acid sequence at least 85% identical to the amino acid sequence of SEQ ID NO: 8.
91. The method of any one of embodiments 72-89, wherein the anti-CD30 antibody of the antibody-drug conjugate comprises a heavy chain variable region comprising an amino acid sequence at least 90% identical to the amino acid sequence of SEQ ID NO: 7 and a light chain variable region comprising an amino acid sequence at least 90% identical to the amino acid sequence of SEQ ID NO: 8.
92. The method of any one of embodiments 72-89, wherein the anti-CD30 antibody of the antibody-drug conjugate comprises a heavy chain variable region comprising an amino acid sequence at least 95% identical to the amino acid sequence of SEQ ID NO: 7 and a light chain variable region comprising an amino acid sequence at least 95% identical to the amino acid sequence of SEQ ID NO: 8.
93. The method of any one of embodiments 72-89, wherein the anti-CD30 antibody of the antibody-drug conjugate comprises a heavy chain variable region comprising the amino acid sequence of SEQ ID NO: 7 and a light chain variable region comprising the amino acid sequence of SEQ ID NO: 8.
94. The method of any one of embodiments 72-89, wherein the anti-CD30 antibody is AC10.
95. The method of any one of embodiments 72-89, wherein the anti-CD30 antibody is cAC10.
96. The method of any one of embodiments 72-95, wherein the antibody-drug conjugate further comprises a linker between the anti-CD30 antibody or antigen-binding portion thereof and the monomethyl auristatin.
97. The method of embodiment 96, wherein the linker is a cleavable peptide linker.
98. The method of embodiment 97, wherein the cleavable peptide linker has a formula: -MC-vc-PAB-.
99. The method of any one of embodiments 72-98, wherein the monomethyl auristatin is monomethyl auristatin E (MMAE).
100. The method of any one of embodiments 72-98, wherein the monomethyl auristatin is monomethyl auristatin F (MMAF).
101. The method of any one of embodiments 72-89, wherein the antibody-drug conjugate is brentuximab vedotin.
102. The method of any one of embodiments 72-101, wherein the antibody-drug conjugate is administered at a dose ranging from of about 1.2 mg/kg of the subject's body weight.
103. The method of any one of embodiments 72-101, wherein the antibody-drug conjugate is administered at a dose ranging from of 1.2 mg/kg of the subject's body weight.
104. The method of any one of embodiments 72-101, wherein the antibody-drug conjugate is administered at a dose of about 0.9 mg/kg of the subject's body weight.
105. The method of any one of embodiments 72-101, wherein the antibody-drug conjugate is administered at a dose of 0.9 mg/kg of the subject's body weight.
106. The method of any one of embodiments 72-105, wherein the antibody-drug conjugate is administered once about every 2 weeks.
107. The method of any one of embodiments 72-105, wherein the antibody-drug conjugate is administered once every 2 weeks.
108. The method of embodiment 106 or 107, wherein the antibody-drug conjugate is administered for four 2-week treatment cycles.
109. The method of any one of embodiments 72-108, wherein the antibody-drug conjugate is administered to the subject by intravenous infusion.
110. The method of embodiment 109, wherein the intravenous infusion is about a 30 minute infusion.
111. The method of any one of embodiments 72-110, wherein the subject has a life expectancy of greater than 9 months prior to administration of the antibody-drug conjugate.
112. The method of any one of embodiments 72-111, wherein the subject has received antiretroviral therapy (ART) for at least 24 weeks prior to administration of the antibody-drug conjugate.
113. The method of embodiment 112, wherein the subject has received ART for at least 12 months prior to administration of the antibody-drug conjugate.
114. The method of embodiment 112, wherein the subject has received ART for at least 24 months prior to administration of the antibody-drug conjugate.
115. The method of any one of embodiments 72-115, wherein the antibody-drug conjugate is administered in combination with ART.
116. The method of any one of embodiments 112-115, wherein the ART is a nucleoside reverse transcriptase inhibitor, non-nucleoside reverse transcriptase inhibitor, protease inhibitor, fusion inhibitor, CCR5 antagonist, integrase inhibitor, post-attachment inhibitor, or pharmacokinetic enhancer.
117. The method of embodiment 116, wherein the ART comprises two or more of a nucleoside reverse transcriptase inhibitor, non-nucleoside reverse transcriptase inhibitor, protease inhibitor, fusion inhibitor, CCR5 antagonist, integrase inhibitor, post-attachment inhibitor, and pharmacokinetic enhancer.
118. The method of embodiment 116, wherein the ART comprises three or more of a nucleoside reverse transcriptase inhibitor, non-nucleoside reverse transcriptase inhibitor, protease inhibitor, fusion inhibitor, CCR5 antagonist, integrase inhibitor, post-attachment inhibitor, and pharmacokinetic enhancer.
119. The method of embodiment 116, wherein the ART comprises four or more of a nucleoside reverse transcriptase inhibitor, non-nucleoside reverse transcriptase inhibitor, protease inhibitor, fusion inhibitor, CCR5 antagonist, integrase inhibitor, post-attachment inhibitor, and pharmacokinetic enhancer.
120. The method of any one of embodiments 112-119, wherein the ART comprises one or more of abacavir, emtricitabine, lamivudine, tenofovir disoproxil fumarate, zidovudine, doravirine, efavirenz, etravirine, nevirapine, rilpivirine, atazanavir, darunavir, fosamprenavir, ritonavir, saquinavir, tipranavir, enfuvirtide, maraviroc, dolutegravir, raltegravir, ibalizumab, and cobicistat.
121. The method of any one of embodiments 112-120, wherein the ART does not comprise a strong CYP3A4 inhibitor.
122. The method of any one of embodiments 112-120, wherein the ART does not comprise a strong P-gp inhibitor.
123. The method of any one of embodiments 72-122, wherein administering the antibody-drug conjugate results an increase in the CD4⁺ T-cell lymphocyte count in the subject to above 200 cells/μL.
124. The method of any one of embodiments 72-123, wherein administering the antibody-drug conjugate results in an increase in the CD4⁺ T-cell lymphocyte count by at least 50 cells/μL relative to the CD4⁺ T-cell lymphocyte count prior to administration.
125. The method of any one of embodiments 72-124, wherein administering the antibody-drug conjugate results an increase in the CD8⁺ T-cell lymphocyte count in the subject relative to the CD8⁺ T-cell lymphocyte count prior to administration.
126. The method of any one of embodiments 72-125, wherein administering the antibody-drug conjugate results in a decrease in the number of Treg cells relative to the number prior to the administration of the antibody-drug conjugate.
127. The method of embodiment 126, wherein the Treg cells are CD4⁺.
128. The method of embodiment 126 or claim 127, wherein the Treg cells are CD30⁺.
129. The method of any one of embodiments 72-128, wherein administering the antibody-drug conjugate results in a decrease in the number of memory T cells relative to the number prior to the administration of the antibody-drug conjugate.
130. The method of embodiment 129, wherein the memory T cells are CD4⁺.
131. The method of embodiment 129 or claim 130, wherein the memory T cells are CD30⁺.
132. The method of any one of embodiments 72-131, wherein the subject has not been administered the antibody-drug conjugate prior to the administration to increase CD4⁺ T-cell lymphocyte count in the subject.
133. The method of any one of embodiments 72-132, wherein the subject is a human.
134. A kit comprising:

(a) a dosage ranging from about 0.1 mg to about 500 mg of an of an antibody-drug conjugate that binds to CD30, wherein the antibody-drug conjugate comprises an anti-CD30 antibody or an antigen-binding fragment thereof conjugated to a monomethyl auristatin or a functional analog thereof or a functional derivative thereof; and

(b) instructions for using the antibody drug conjugate according to the method of any one of embodiments 72-133.

135. Use of an antibody-drug conjugate that binds to CD30 for the manufacture of a medicament for use in the method of any one of embodiments 72-133.
136. An antibody-drug conjugate that binds to CD30 for use in the method of any one of embodiments 72-133.

The invention will be more fully understood by reference to the following examples. They should not, however, be construed as limiting the scope of the invention. It is understood that the examples and embodiments described herein are for illustrative purposes only and that various modifications or changes in light thereof will be suggested to persons skilled in the art and are to be included within the spirit and purview of this application and scope of the appended claims.

### EXAMPLES

### Example 1: Effect of Brentuximab Vedotin (BV) on T Cell Viability

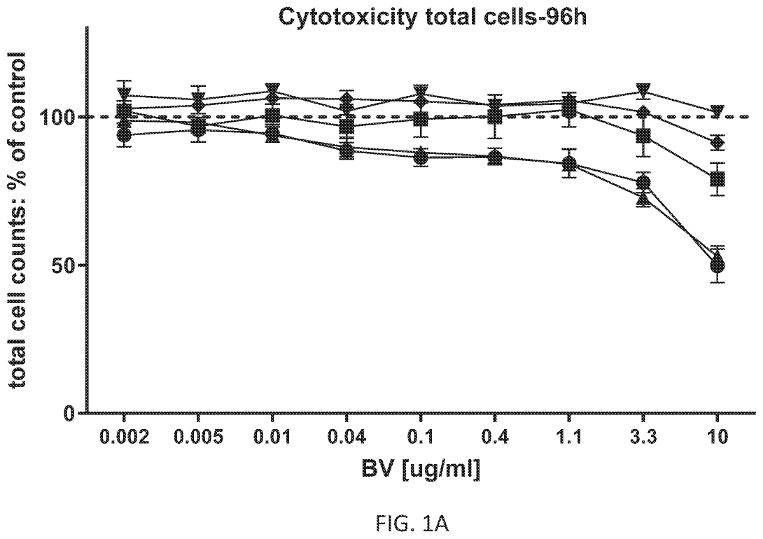

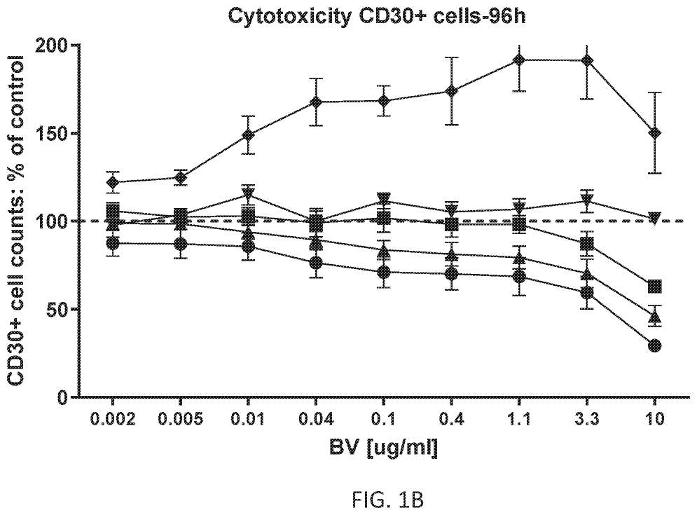

To evaluate the effect of BV on activated T cell viability, naïve, memory, and Treg subsets were driven to proliferate in vitro in the presence of BV or control antibody-drug conjugate (ADC). Briefly, T cell subsets were mixed with CD3/CD28 beads (4:1)+IL-2 10 ng/ml in RPMI 10% FCS and distributed −2.0×10⁴ cells/well into a 96-well round-bottom plate. A titration of BV or control IgG-MMAE was added to replicate wells, for a final volume of 200 μl, and plates were incubated at 37° C. for 4 days. On the final day of the assay, cells were stained with Zombie® Aqua Viability Dye and a non-competing monoclonal μCD30-PE (Biolegend) for FACS analysis. As shown in FIG. 1A, BV drove a dose-dependent reduction in total viable Treg and memory CD4 T cell numbers (n=4). As populations of T cell subsets displayed heterogeneous CD30 expression during activation, and BV selectively targets CD30 expressing cells, numbers of viable CD30⁺ cells were determined at the end of the assay. Consistent with the observed effects on total Treg and memory CD4 T cells from culture, BV showed enhanced depletion of CD30⁺ cells of these subtypes (FIG. 1B). Cell counts are shown as the percent of untreated control.

### Example 2: CD30 and CD30L Expression in T Cell Populations Over Time

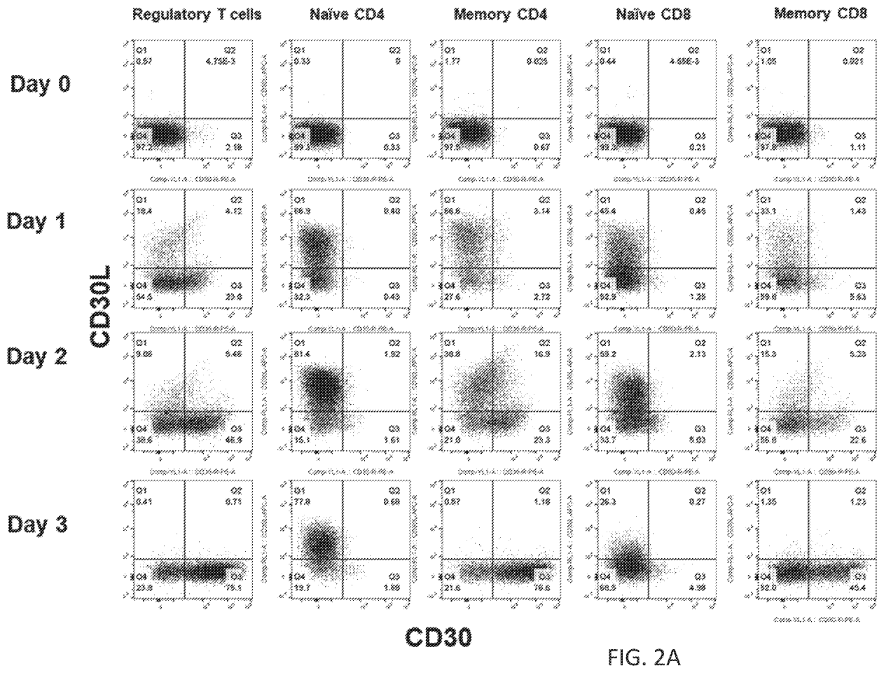

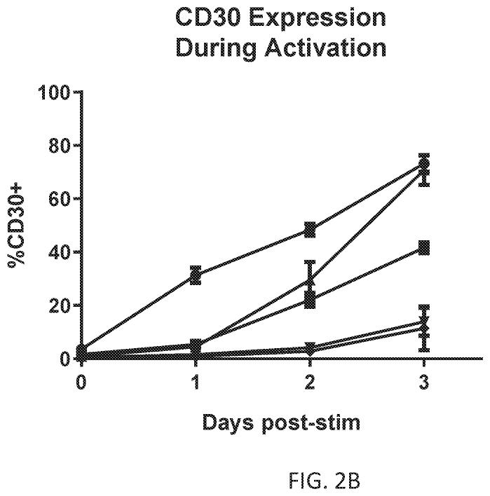

CD30 has been described to be upregulated on T cells, and specifically memory T cells, around 48 hours after activation, while less is known about receptor-engaging CD30L expression. To evaluate the relative expression kinetics of CD30 and CD30L on T cell subsets following activation, enriched populations of Naïve, Memory, and Treg cells were synchronously activated with CD3/CD28 beads (4:1)+IL-2 10 ng/ml in RPMI 10% FCS. Each day, CD30 and CD30L expression was monitored by flow cytometry. FIG. 2A shows a representative flow plot of T cell subsets CD30 and CD30L expression over a 3-day activation time-course. T regulatory cells showed rapid expression of CD30 following activation with CD3/CD28 with minimal expression of CD30L, while other T cell subsets tended to have robust surface CD30L expression prior to CD30 (n=4) (FIG. 2B). Loss of CD30L coincided with increased expression of CD30 on all T cell subsets and may point to a gradual self-extinguishing transition from dominant-ligand to dominant-receptor surface expression. These data suggest that heightened CD30 expression observed on Tregs may be related to an intrinsic ability to more rapidly produce surface CD30, or to a lack of co-expressed ligand allowing for greater CD30 surface expression. Increased CD30 expressed by Tregs and memory CD4 T cells following activation may enhance target-mediated drug delivery by BV.

### Example 3: Rhodamine 123 Efflux Over Time in T Cell Populations

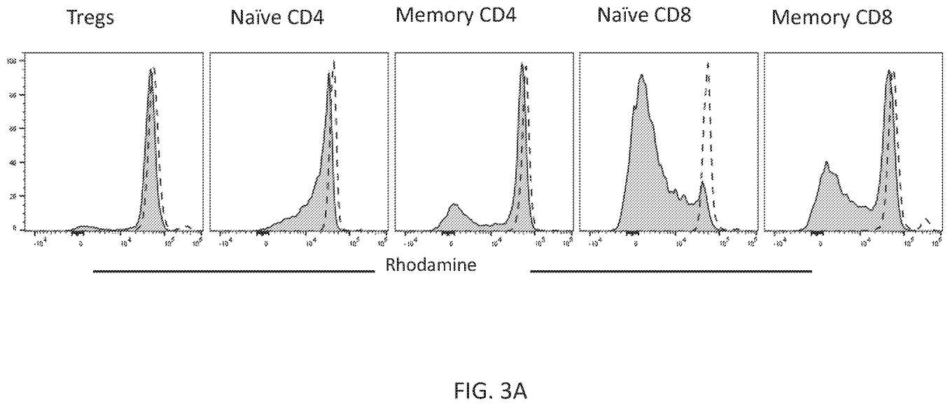

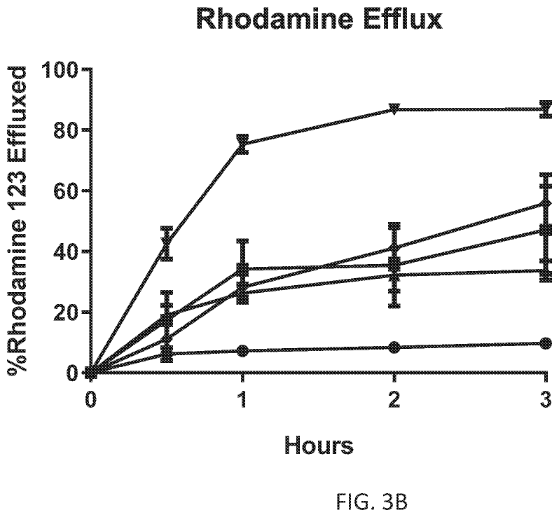

Sensitivity of cells to many chemotherapies, including MMAE, is influenced by cell-intrinsic drug efflux activity. T cell subsets were evaluated for relative efflux pump activity using a rhodamine 123 efflux assay, following manufacturer's protocol (Chemicon International, Multidrug Resistance Direct Dye Efflux Assay). Enriched T cell populations were loaded with rhodamine 123, incubated in a 37° C. water bath, and measured for loss of fluorescence over a 5-hour time-course by flow cytometry. T regulatory cells failed to efflux rhodamine-123 over a 3-hour time-course while other T cell subsets showed modest to high permeability glycoprotein (Pgp) driven efflux capacity (FIG. 3A and FIG. 3B). MMAE is a known Pgp substrate. The inability of Tregs to efficiently efflux rhodamine suggests a propensity for higher MMAE accumulation following treatment with BV, and may contribute to increased potency on this T cell subset.

### Example 4: Effects of Treating T Cell Populations with Free Monomethyl Auristatin E (MMAE)

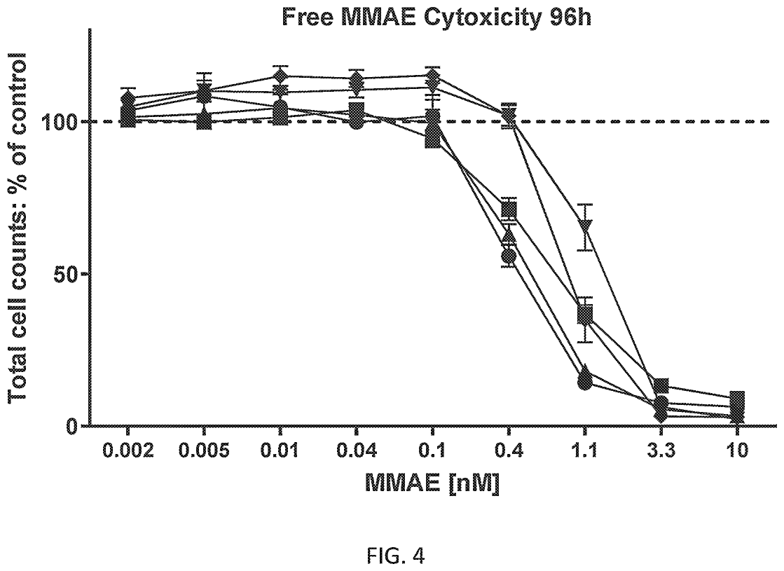

To evaluate the effect of free MMAE on the various T cell populations, naïve (CD45RA+, CD45RO−) and memory (CD45RA−, CD45RO+) CD4 and CD8 T cells and CD25^{hi} CD127^{lo} T regulatory cells were enriched from healthy donor leukopaks and activated with CD3/CD28 beads and IL-2 (10 ng/ml) in round-bottom 96-well tissue culture plates for 4 days with a titration of free monomethyl auristatin E (MMAE). As shown in Table 1 and in FIG. 4, memory T cells and Tregs showed similar sensitivity to free MMAE in vitro. Data are represented as cell counts relative to untreated control.

TABLE 1

| T cell population | IC₅₀ (nM) |
|---|---|
| Treg | 0.43 |
| Naïve CD4 | 1.13 |
| Memory CD4 | 0.55 |
| Naïve CDS | 1.96 |
| Memory CDS | 0.77 |

### Example 5: A Phase I/I Clinical Study Evaluating the Addition of Brentuximab Vedotin to Combined Antiretroviral (cART) in Subjects with Human Immunodeficiency Virus (HIV)

This is an open label, multi-center trial of brentuximab vedotin in combination with cART in subjects with HIV with inadequate CD4 lymphocyte count despite cART. The efficacy, safety and tolerability of brentuximab vedotin in combination with cART in subjects with HIV is evaluated herein.

Despite highly active cART, viral reservoirs persist in infected cells in individuals on cART. There are few therapeutic strategies to decrease the number of these persistently infected cells, and novel approaches to eliminate or reduce HIV reservoir burden are urgently needed. T regulatory cells (Tregs) have been implicated as a possible reservoir for HIV. Tregs have been demonstrated to express CD30. In subjects with CD30+ lymphomas, treatment with brentuximab vedotin resulted in a reduction in Tregs. This study will evaluate the impact of brentuximab vedotin on the HIV viral reservoir as measured by changes in lymphocyte counts and HIV viral load.

### Methods

This is an open label, multi-center trial of brentuximab vedotin in combination with cART in subjects with HIV with inadequate CD4 lymphocyte counts despite optimal cART. A total of 30 subjects will be enrolled in the study over a period of 24 months. All enrolled subjects will have had a confirmed diagnosis of HIV and inadequate CD4 lymphocyte counts despite optimal cART. Ten subjects each will be treated with brentuximab vedotin at doses of 0.3 mg/kg, 0.6 mg/kg or 0.9 mg/kg, by intravenous (IV) infusion over approximately 30 minutes on Day 1 of each 21-day cycle for a total of 6 cycles. Subjects will also continue on their prior cART regimen. Efficacy assessments will be conducted during the study, at the end of Cycle 1, Cycle 2 and end of treatment (EOT), and every 3 months in follow up for 1 year. Safety will be evaluated by collecting information on adverse events and using local labs. Subjects enrolled in the trial are 18 years Inclusion criteria and exclusion criteria for subjects enrolled in the trial are shown in Table 2.

TABLE 2
List of inclusion and exclusion criteria

| | | |
|---|---|---|
| Inclusion | 1. | Males and females, age ≥18 years; |
| Criteria | 2. | HIV-1 seropositive, with documented HIV-1 infection by official, |
| | | signed, written history (e.g., Laboratory report); |
| | 3. | Receiving combination antiretroviral therapy (cART) for at least 24 |
| | | weeks |
| | 4. | CD4 lymphocyte count <200 cells/mm³ |
| | 5. | Plasma HIV-1 RNA ≥ 1000 copies/mL at the Screening Visit and |
| | | documented detectable viral load (HIV-1 RNA >2,00 copies/ml) within |
| | | the last 3 months prior to the Screening Visit; |
| | 6. | Have a life expectancy that is >9 months; |
| | 7 | Laboratory values at Screening of: |
| | | Absolute neutrophil count (ANC) ≥750/mm3; |
| | | Hemoglobin (Hb) ≥10.5 gm/dL (male) or ≥9.5 gm/dL (female); |
| | | Platelets ≥75,000/mm3; |
| | | Serum alanine transaminase (SGPT/ALT) <2.5× upper limit of |
| | | normal (ULN); |
| | | Seram aspartate transaminase (SGOT/AST) <2.5× ULN; |
| | | Bilirubin (total) <2.5× ULN unless Gilbert's disease is present |
| | | or subject is receiving atazanavir in the absence of other |
| | | evidence of significant liver disease; and |
| | 8. | Creatinine <1.5× ULN |
| | | Clinically normal resting 12-lead electrocardiogram (ECG) at the |
| | | Screening Visit or, if abnormal, considered not clinically significant by |
| | | the Principal Investigator. |
| | 9. | Both male and female patients and their partners of childbearing |
| | | potential must agree to use 2 medically accepted methods of |
| | | contraception (e.g., barrier contraceptives [male condom, female |
| | | condom, or diaphragm with a spermicidal gel], hormonal contraceptives |
| | | [implants, injectables, combination oral contraceptives, transdermal |
| | | patches, or contraceptive rings], and intrauterine devices) during the |
| | | course of the study (excluding women who are not of childbearing |
| | | potential and men who have been sterilized). Females of childbearing |
| | | potential must have a negative serum pregnancy test at the Screening |
| | | Visit and negative urine pregnancy test prior to receiving the first dose |
| | | of study drug; and |
| | 10. | Willing and able to participate in all aspects of the study, including use |
| | | of IV medication, completion of subjective evaluations, attendance at |
| | | scheduled clinic visits, and compliance with all protocol requirements as |
| | | evidenced by providing written informed consent. |
| Exclusion | 1. | Active malignancy or history of active malignancy within 24 months |
| Criteria | 2. | Any currently active AIDS-defining illness per Category C conditions |
| | | according to the Center for Disease Control (CDC) Classification |
| | | System for HIV Infection, with the following exceptions: local |
| | | cutaneous Kaposi's sarcoma, wasting syndrome due to HIV or any other |
| | | AIDS-defining illness for which no therapeutic treatment is required OR |
| | | the required treatment is not included in the list of prohibited |
| | | medications; |
| | 3. | Subjects with baseline liver disease including active Hepatitis B or C |
| | | infection or any other active infection secondary to HIV requiring acute |
| | | therapy; |
| | 4. | Any ≥Grade 3 laboratory abnormality according to the division of AIDS |
| | | grading scale; |
| | 5. | Unexplained fever or clinically significant illness within 2 weeks prior |
| | | to the first dose of study drug; |
| | 6. | Any vaccination within 2 weeks prior to the first dose of study drug; |
| | 7. | Any immunomodulating therapy (excluding pre-medication steroid) or |
| | | systemic chemotherapy within 4 weeks prior to the Screening Visit; |
| | 8. | Any radiation therapy within 4 weeks prior to the Screening Visit; |
| | 9. | Participation in an experimental drug trial(s) within 4 weeks prior to the |
| | | Screening Visit; |
| | 10. | Any prior exposure to brentuximab vedotin; |
| | 11. | Females who are pregnant, lactating, or breastfeeding, or who plan to |
| | | become pregnant during the study; and |
| | 12. | Any significant diseases (other than HIV-1 infection) or clinically |
| | | significant findings that, in the Investigator's judgment, would |
| | | potentially compromise study compliance or the ability to evaluate |
| | | safety/efficacy. |

The primary objectives of the study are 1) to assess the safety and tolerability of brentuximab vedotin plus cART and 2) to assess the impact of brentuximab vedotin plus cART on CD4, CD8, and Treg cell subsets. The secondary objective is to assess the impact of brentuximab vedotin plus cART on HIV viral load. Subjects will be evaluated for changes in CD4, CD8, Treg cells and other lymphocyte subsets as well as for changes in HIV viral load. Safety assessments will include the surveillance and recording of adverse events, physical examination findings, and laboratory tests.

### Example 6: A Phase II Clinical Study Evaluating the Addition of Brentuximab Vedotin to Combined Antiretroviral (cART) in Subjects with Human Immunodeficiency Virus (HIV)

This is a phase 2, open-label, multicenter, randomized trial to assess the effect of brentuximab vedotin (ADCETRIS®) in subjects with human immunodeficiency virus (HIV) who have inadequate immune reconstitution despite viral suppression with combined antiretroviral therapy (ART).

One of the most significant risk factors for all-cause mortality in persons living with human immunodeficiency virus (HIV)/acquired immunodeficiency syndrome (AIDS) (PLWHA) is an incomplete CD4⁺ T-cell recovery, specifically levels below 200 cells/μL. Poor CD4⁺ T-cell count recovery in PLWHA not only increases rates of all-cause mortality, but also affects the rate of AIDS-defining malignancies (NHL, Kaposi sarcoma, and cervical cancer), non-AIDS defining malignancies, infectious complications, and has been attributed to an increased risk of cardiovascular events.

While the introduction of ART decreased the incidence of all-cause mortality, cancer incidences, and infectious complications, not all PLWHA experience an increase in CD4⁺ T-cell count when taking anti-HIV medications. Depending on the study, 15% to 30% of all patients taking ART will achieve HIV-1 viral undetectability in peripheral blood with little to no increase in CD4 T-cell count. These patients are called immunological nonresponders (INRs), and the failure to increase CD4⁺ T-cell counts despite long-term viral suppression while on ART may be associated with several risk factors. These may include but are not limited to increasing age, lower initial CD4⁺ T-cell count, and a longer duration from the start of ART to the beginning of the virally-suppressed period.

Brentuximab vedotin is a CD30-directed antibody-drug conjugate (ADC) consisting of 3 components: 1) the chimeric IgG1 antibody cAC10, specific for human CD30; 2) the microtubule-disrupting agent monomethyl auristatin E (MMAE); and 3) a protease-cleavable linker that covalently attaches MMAE to cAC10. Targeted delivery of MMAE to CD30-expressing tumor cells is the primary mechanism of action of brentuximab vedotin. Binding of MMAE to tubulin disrupts the microtubule network within the cell, subsequently inducing cell cycle arrest and apoptotic death of the cell. Other nonclinical studies suggest additional contributory mechanisms of action, including antibody-dependent cellular phagocytosis; bystander effects on nearby cells in the tumor microenvironment due to released MMAE; and immunogenic cell death due to endoplasmic reticulum stress which drives exposure of immune activating molecules that can promote a T-cell response.

### Methods

This is a phase 2, open-label, multicenter, randomized study designed to evaluate the effect of brentuximab vedotin in subjects with HIV who have inadequate immune reconstitution despite viral suppression with ART. Subjects must have documentation of HIV-1 infection and be immunological nonresponders (INR) as defined in the inclusion criteria. Subjects must be on ART and have had an HIV viral load <50 copies/mL for at least 24 months. Eligible subjects must also have a CD4⁺ T-cell lymphocyte count between 51 to 200 cells/μL at screening. Strong CYP3A4 or P-gp inhibitors are excluded; however, subjects taking these medications may switch to a permitted ART regimen following a 7-day washout and still be eligible for enrollment. All subjects must remain on a permitted ART regimen while receiving brentuximab vedotin.

Approximately 60 subjects will be enrolled and randomized in a 2:1 ratio to either the treatment arm (Arm 1) or the control arm (Arm 2) as follows:

Arm 1: brentuximab vedotin 1.2 mg/kg+ART

Arm 2: ART only (option to crossover at Week 24 and receive brentuximab vedotin)

Subjects in Arm 1 will receive brentuximab vedotin 1.2 mg/kg q2 wk for 4 doses. If CD4⁺ T-cell count has not risen above 200 cells/μL following 4 doses of brentuximab vedotin, subjects in Arm 1 may receive up to an additional 2 doses (at Week 8 and Week 10), following discussion and agreement between the investigator and medical monitor.

After 24 weeks, subjects in the control arm (Arm 2) may receive brentuximab vedotin at the same dose and schedule as in Arm 1.

Any subject (Arm 1 or Arm 2) who receives brentuximab vedotin and experiences an increase in CD4⁺ T-cell count above 200 cells/μL with a minimum increase of 50 cells/μL, that subsequently decreases to below 200 cells/μL within 24 to 48 weeks after the last dose may be retreated (once only) for up to an additional 2 doses of brentuximab vedotin 1.2 mg/kg q2 wk if all eligibility criteria continue to be met. Subjects who are retreated will be followed for an additional 12 months after retreatment to assess the duration of response.

Safety will be continuously monitored throughout the study by the sponsor and the Safety Monitoring Committee (SMC), with consideration for enrollment halt if the incidence and/or the severity of toxicity leads to a risk-benefit assessment that is unacceptable to the study population.

The SMC will consider whether subjects already receiving treatment are allowed to continue, if modifications to the protocol are needed to continue enrollment, or if the study should be terminated.

Inclusion criteria and exclusion criteria for subjects to be enrolled in the trial are shown in Table 3.

TABLE 3
List of inclusion and exclusion criteria

| | | |
|---|---|---|
| Inclusion | 1. | Male or female aged ≥18 years. |
| criteria | 2. | HIV-1 seropositive, with documentation of infection by means of |
| | | one of the following: |
| | | Documentation of diagnosis in the subject's medical record |
| | | made by a licensed physician |
| | | Documentation of receipt of ART as prescribed by a licensed |
| | | physician, excluding pre-exposure prophylactic settings |
| | | HIV-1 ribonucleic acid (RNA) detection by an FDA-approved |
| | | HIV-1 RNA assay demonstrating >20 RNA copies/mL |
| | | Any FDA-approved HIV screening antibody and/or HIV |
| | | antibody/antigen combination assay confirmed by a second |
| | | FDA-approved HIV assay. |
| | 3. | Immunological nonresponder, defined as: |
| | | Has been on ART with an HIV viral load <50 copies/mL for at |
| | | least 24 months. |
| | | Note: HIV viral load (plasma HIV-1 RNA) will be measured |
| | | by quantitative polymerase chain reaction (PCR) assay using |
| | | an FDA-approved test. Subjects who had a detectable viral |
| | | load <200 copies/mL within the previous 24 months that |
| | | subsequently became undetectable the next time viral load was |
| | | assessed are eligible for enrollment if 1) the detectable viral |
| | | load did not occur within 6 months of Screening and 2) viral |
| | | load elevations did not occur more than twice over the |
| | | 24-month period. |
| | | Note: The ART regimen may be at the discretion of the |
| | | investigator with the exception of strong CYP3A4 or P-gp |
| | | inhibitors (e.g., ritonavir, cobicistat), which are prohibited. |
| | | Subjects may switch to a permitted ART regimen following a |
| | | 7-day washout period and still be considered for enrollment. |
| | | Has a CD4+ T-cell lymphocyte count between 51 to 200 |
| | | cells/μL. |
| | 4. | Life expectancy of >9 months. |
| | 5. | Laboratory values at the Screening Visit of: |
| | | Absolute neutrophil count ≥1000/mm3. |
| | | Hemoglobin ≥10.5 g/dL (male) or ≥9.5 g/dL (female). |
| | | Platelets ≥75,000/mm3. |
| | | Alanine transaminase (ALT) and aspartate transaminase (AST) |
| | | <3.0× upper limit of normal (ULN). |
| | | Bilirubin (total) <1.5× ULN or ≤3× ULN for subjects with |
| | | Gilbert's disease. If elevated bilirubin is thought to be |
| | | secondary to ART, total bilirubin must be ≤3.5 mg/dL, direct |
| | | bilirubin must be normal, and AST and ALT must be ≤3× |
| | | ULN. |
| | | Creatinine ≤1.5× ULN; if creatinine is >1.5 mg/dL, estimated |
| | | glomerular filtration rate (GFR) must be ≥60 mL/min/1.73m² |
| | | using the Modification of Diet in Renal Disease (MDRD) |
| | | study equation as applicable. |
| | 6. | A Karnofsky performance status score ≥50%. |
| | 7. | Clinically normal resting 12-lead electrocardiogram (ECG) at the |
| | | Screening Visit or, if abnormal, considered not clinically |
| | | significant by the investigator. |
| | 8. | Negative for hepatitis B, or if infected with hepatitis B, receiving |
| | | anti-hepatitis B therapy. All participants will be required to be |
| | | screened for hepatitis B. Per IDSA and AASD guidelines, those |
| | | participants that show no immunity, defined by the lack of |
| | | Hepatitis B surface antibody, and show evidence of chronic |
| | | infection (i.e., HBsAg+, HBcore+, HBsAB−) will be required to be |
| | | on anti-hepatitis B therapy during the study in order to be eligible. |
| | | Subjects will be permitted to enroll in the study if liver function |
| | | tests are normal and there is no evidence of cirrhosis. The exact |
| | | hepatitis B therapy will be at the discretion of the infection disease |
| | | specialist or investigator; however, all subjects who present with |
| | | acute hepatitis B or show normal transaminases and are HBsAg+ |
| | | and IgM+ for hepatitis core antigen will not be eligible for |
| | | enrollment. |
| | 9. | Subjects with a history of hepatitis C virus (HCV) are only eligible |
| | | if they completed therapy for HCV and show sustained virologic |
| | | remission. |
| | 10. | Subjects of childbearing potential must have a negative serum or |
| | | urine beta human chorionic gonadotropin (β-hCG) pregnancy test |
| | | result within 7 days prior to the first dose of brentuximab vedotin. |
| | | Subjects with false positive results and documented verification |
| | | that the subject is not pregnant are eligible for participation. |
| | | Subjects of non-childbearing potential are those who are |
| | | postmenopausal >1 year or who have had a bilateral |
| | | oophorectomy or hysterectomy. |
| | 11. | If sexually active in a way that could result in pregnancy, subjects |
| | | of childbearing potential and subjects who can father children and |
| | | have partners of childbearing potential must agree to use 2 |
| | | effective contraception methods during the study and for 6 months |
| | | following the last dose of study drug. |
| | 12. | The subject or the subject's legally acceptable representative must |
| | | provide written informed consent. |
| Exclusion | 1. | Any currently active AIDS-defining illness per Category C |
| criteria | | conditions according to the CDC Classification System for HIV |
| | | Infection, with the following exceptions: limited cutaneous |
| | | Kaposi's sarcoma not currently requiring systemic therapy, |
| | | wasting syndrome due to HIV or any other AIDS-defining illness |
| | | for which no therapeutic treatment is required OR the required |
| | | treatment is not included in the list of prohibited medications. |
| | 2. | Acute liver disease or any other active infection secondary to HIV |
| | | requiring acute therapy. Subjects with hepatitis B or C must meet |
| | | normal liver function test criteria to be eligible for enrollment. |
| | 3. | Any ≥Grade 3 nonhematologic laboratory abnormality according |
| | | to the NCI CTCAE version 5.0. |
| | 4. | Unexplained fever or clinically significant illness within 2 weeks |
| | | prior to the first dose of study drug. |
| | 5. | History of progressive multifocal leukoencephalopathy (PML). |
| | 6. | History of John Cunningham virus (JCV) identified in |
| | | cerebrospinal fluid. |
| | 7 | Cirrhosis secondary to any cause. |
| | 8. | Any attenuated vaccine within 2 weeks prior to the first dose of |
| | | study drug. |
| | 9. | Any immunomodulating therapy (excluding premedication |
| | | steroid) within 4 weeks prior to the Screening Visit. |
| | 10. | Prior malignancy within 2 years other than cutaneous basal cell or |
| | | squamous cell carcinoma, carcinoma in situ of the cervix, anal |
| | | intraepithelial neoplasia, or cutaneous Kaposi's sarcoma. Subjects |
| | | with prior malignancies must have completed all therapy at least 2 |
| | | years prior to enrollment with no evidence of disease since |
| | | completion of therapy. |
| | 11. | Documented history of a cerebral vascular event (stroke or |
| | | transient ischemic attack), unstable angina, myocardial infarction, |
| | | congestive heart failure, or cardiac symptoms consistent with New |
| | | York Heart Association Class III-IV within 6 months prior to their |
| | | first dose of brentuximab vedotin. |
| | 12. | Grade 2 or higher peripheral sensory or motor neuropathy at |
| | | baseline. |
| | 13. | Participation in an experimental drug trial(s) within 4 weeks prior |
| | | to the Screening Visit. |
| | 14. | Any prior exposure to brentuximab vedotin. |
| | 15. | Known hypersensitivity to any excipient contained in the drug |
| | | formulation of brentuximab vedotin. |
| | 16. | Females who are pregnant, lactating, or breastfeeding, or who plan |
| | | to become pregnant during the study. |
| | 17. | Any significant diseases (other than HIV-1 infection) or clinically |
| | | significant findings that, in the investigator's judgment, would |
| | | potentially compromise study compliance or the ability to evaluate |
| | | safety and effect. |

Brentuximab vedotin is a sterile, preservative free, white to off-white lyophilized cake or powder supplied by Seattle Genetics in single-use vials for reconstitution for IV administration. Each vial of the product contains brentuximab vedotin, trehalose, sodium citrate, and polysorbate 80.

Brentuximab vedotin vials are provided via single-use containers. Brentuximab vedotin should be reconstituted with the appropriate amount of Sterile Water for Injection, United States Pharmacopeia (USP) or equivalent. The vial should be gently swirled until the contents are completely dissolved. The vial must not be shaken. The reconstituted drug product should be inspected visually for any particulate matter and discoloration. The required volume of reconstituted drug product should be diluted into an infusion bag. The bag should be gently inverted to mix the solution. The bag must not be shaken. Prior to administration, the reconstituted and diluted drug product should be inspected visually for any particulate matter and discoloration.

Brentuximab vedotin will be administered by IV infusion given over approximately 30 minutes. The dose for all subjects receiving brentuximab vedotin is 1.2 mg/kg q2 wk for 4 doses. If CD4⁺ T-cell count has not risen above 200 cells/μL following 4 doses of brentuximab vedotin, subjects may receive up to an additional 2 doses, following discussion and agreement between the investigator and medical monitor. The dosing regimen, including the dose level, dosing frequency, and dosing duration, may be modified as recommended by the SMC based on a review of the available safety data.

In the absence IRRs, the infusion rate for all subjects should be calculated in order to achieve a 30-minute infusion period. Brentuximab vedotin must not be administered as an IV push or bolus. Brentuximab vedotin should not be mixed with other medications.

Weight-based dosing is based on subject actual body weight. Doses must be adjusted for subjects who experience a ≥10% change in weight from baseline. Subject weight must be measured during all relevant assessment windows as described in the schedule of events. Other dose adjustments for changes in body weight are permitted per institutional standard. Rounding is permissible within 5% of the nominal dose. An exception to weight-based dosing is made for subjects weighing greater than 100 kg; doses will be based on 100 kg for these individuals. The maximum dose calculated per dose in this study is 120 mg.

Table 4 describes the recommended dose modifications for study treatment-associated toxicity. Doses reduced for treatment-related toxicity should not be re-escalated without discussion with the sponsor.

TABLE 4
Recommended dose modifications for brentuximab vedotin-associated toxicity

| Toxicity | Grade 1 | Grade 2 | Grade 3 | Grade 4 |
|---|---|---|---|---|
| Peripheral | Continue at same | Withhold until | Discontinue | Discontinue |
| Neuropathy | dose level | toxicity resolves to | treatment^{g} | treatment^{g} |
| | | Grade 1 or | | |
| | | baseline, then | | |
| | | resume treatment | | |
| | | at 0.9 mg/kg^{a,f} | | |
| Non-hematologic | Continue at same | Continue at same | Withhold dose until | Withhold dose until |
| (except peripheral | dose level | dose level | toxicity is <Grade 2 or | toxicity is ≤Grade 2 |
| neuropathy) | | | has returned to baseline, | or has returned to |
| | | | then resume treatment at | baseline, then reduce |
| | | | the same dose level^{b}. | dose to 0.9 ing/kg and |
| | | | | resume treatment, or |
| | | | | discontinue at the |
| | | | | discretion of the |
| | | | | investigator ^{a,b,c} |

| | | | |
|---|---|---|---|
| Hematologic^{d} | Continue at same | Continue at same | Withhold until toxicity resolves to ≤Grade 2 or |
| | dose level | dose level | baseline, then resume treatment at the same dose |
| | | | level^{e}. For Grade 3 or 4 neutropenia, growth |
| | | | factor support (G-CSF or GM-CSF) should be |
| | | | considered for subsequent doses. If Grade 4 |
| | | | neutropenia recurs despite growth factor support, |
| | | | consider discontinuation or dose reduction to 0.9 |
| | | | mg/kg. |

^{a}Dose reductions below 0.9 mg/kg are allowed after discussion with the medical monitor. Toxicities may also be managed with dose delays after discussion with the medical monitor.
^{b}Subjects who develop Grade 3 or 4 electrolyte laboratory abnormalities may continue study treatment without interruption.
^{c}Treatment should be discontinued for subjects who experience Grade 4 infusion-related reactions.
^{d}Support with blood product transfusions allowed per institutional standard of care.
^{e}Subjects who develop Grade 3 or 4 lymphopenia may continue study treatment without interruption.
^{f}In case of drag withholding due to Grade 2 peripheral neuropathy, restarting the study treatment may occur after a discussion with the medical monitor.
^{g}Grade 3 or 4 peripheral neuropathy will be followed until resolution, return to baseline, or study closure.

Routine premedication should not be administered for the prevention of IRRs prior to the first dose of brentuximab vedotin. However, subjects who experience a Grade 1 or Grade 2 IRR may receive subsequent brentuximab vedotin infusions with premedication. Subjects who experience a Grade 3 or Grade 4 IRR may potentially receive additional treatment with brentuximab vedotin at the discretion of the investigator after discussion with the sponsor.

This study will evaluate the safety and effect of brentuximab vedotin in subjects with HIV. Specific objectives and corresponding endpoints for the study are summarized in Table 5.

TABLE 5
Objectives and corresponding endpoints

| Primary Objective | Corresponding Primary Endpoint |
|---|---|
| To assess the ability of brentuximab vedotin to increase | Proportion of subjects at Week 16 with CD4⁺ T-cell |
| CD4⁺ T-cell lymphocyte count above 200 cells/μL at | lymphocyte count >200 cells/μL, with a minimum |
| Week 16, with a minimum increase of 50 cells/μL | increase of 50 cells/μL |
| Secondary Objectives | Corresponding Secondary Endpoints |
| To assess the change in CD4⁺ T-cell lymphocyte counts | Change from baseline at Weeks 2, 4, 6, 8, 16, 24, 32, |
| following treatment with brentuximab vedotin | and 48 in CD4+ T-cell lymphocyte counts |
| To assess the ability of brentuximab vedotin to increase | Proportion of subjects at Weeks 2, 4, 6, 8, 16, 24, 32, |
| CD4⁺ T-cell lymphocyte count at time points other than | and 48 with CD4⁺ T-cell lymphocyte count |
| Week 16 | >200 cells/μL |
| | Proportion of subjects at Weeks 2, 4, 6, 8, 24, 32, and |
| | 48 with CD4+ T-cell lymphocyte count |
| | >200 cells/μL, with a minimum increase of |
| | 50 cells/μL |
| To assess the effect of brentuximab vedotin on CD8⁺ T- | Change from baseline at Weeks 2, 4, 6, 8, 16, 24, 32, |
| cell count and CD4:CD8 ratio | and 48 in CD8⁺ T-cell lymphocyte counts and |
| | CD4:CD8 ratio |
| To assess the duration of CD4⁺ T-cell lymphocyte | Duration of CD4⁺ T-cell lymphocyte count increases |
| count increases induced by brentuximab vedotin | >200 cells/μL |
| | Duration of CD4⁺ T-cell lymphocyte count increases |
| | >200 cells/μL with a minimum increase of 50 |
| | cells/μL |
| To assess the effect of brentuximab vedotin on Treg | Change from baseline at Weeks 2, 4, 8, 16, and 24 in |
| and other T-cell subsets | Treg and other T-cell subsets |
| To assess the effect of brentuximab vedotin on HIV | Proportion of subjects at Weeks 2, 4, 6, 8, 16, 24, 32, |
| viral load | and 48 with HIV viral load <50 copies/mL |
| To assess the rate of opportunistic infections and death | Proportion of subjects with fatal or non-fatal |
| from any cause | AIDS-related opportunistic disease or death from any |
| | cause |
| To assess the safety and tolerability of brentuximab | Type, incidence, severity, seriousness, and relatedness |
| vedotin in subjects with HIV | of adverse events |
| | Type, incidence, and severity of laboratory |
| | abnormalities |
| Exploratory Objectives | Corresponding Exploratory Endpoints |
| To assess the potential association between CD30 | Percentage and absolute number of CD30⁺ Tregs or |
| expression level (in Treg and other T-cell subsets) and | other CD30⁺ T cells at baseline and brentuximab |
| CD4⁺ T-cell reconstitution | vedotin-induced increase in CD4⁺ T-cell count |
| To assess the effect of brentuximab vedotin on | HIV-1 RNA in plasma (copies/mL) and PBMC |
| measures of HIV persistence | (copies/million cells) |
| To assess the effect of brentuximab vedotin on T-cell | Antigen-specific T-cell response and/or serum/plasma |
| function and/or cytokine production | cytokine levels at baseline and different time points |
| | post brentuximab vedotin administration |
| To assess the potential association between soluble | Soluble CD30 in serum or plasma at baseline and |
| CD30 and CD4⁺ T-cell reconstitution by brentuximab | different time points post brentuximab vedotin |
| vedotin | administration |
| To assess the PK and immunogenicity of brentuximab | Brentuximab vedotin concentrations and incidence of |
| vedotin in the HIV setting | ADA to brentuximab vedotin |
| To assess the effect of brentuximab vedotin on health- | Total and domain scores of the WHOQOL-HIV |
| related quality of life (HRQoL) | BREF and change from baseline. |

### Stopping Criteria

Enrollment in the entire study will be halted by the sponsor if the overall benefit-risk balance is considered negative. Safety will be continuously monitored throughout the study by the sponsor and the SMC, with consideration for enrollment halt if the incidence and/or the severity of toxicity leads to a risk-benefit assessment that is unacceptable to the study population. The SMC will consider whether subjects already receiving treatment are allowed to continue, if modifications to the protocol are needed to continue enrollment, or if the study should be terminated. The SMC will provide recommendations. Final decisions will be made by the sponsor.

The dose-limiting toxicity (DLT) evaluation period will be the first 4 weeks following the first dose of brentuximab vedotin. DLTs will also be evaluated for the first 9 subjects as part of the safety run-in period. A DLT is defined as any of the following during the DLT evaluation period if assessed by the investigator to be related to brentuximab vedotin treatment. Grading will be according to the National Cancer Institute Common Terminology Criteria for Adverse Events (NCI CTCAE), Version 5.0:
Grade 5 toxicity
Grade 4 neutropenia lasting longer than 7 days
Grade 4 thrombocytopenia, or Grade 3 thrombocytopenia with clinically significant bleeding that requires a platelet transfusion
Grade 4 anemia
Grade ≥3 febrile neutropenia
Any Grade ≥3 nonhematologic toxicity (not laboratory), with the following exceptions: Grade 3 allergic reaction, fatigue, asthenia, anorexia, fever, constipation, nausea, vomiting, or diarrhea that resolves within 72 hours, with or without intervention
Any Grade 3 or Grade 4 nonhematologic laboratory value if:
   Clinically significant medical intervention is required to treat the subject, or the abnormality leads to hospitalization, or the abnormality persists for ≥7 days
   The abnormality results in a drug-induced liver injury (DILI)
   Exceptions are clinically nonsignificant, treatable, or reversible laboratory abnormalities including liver function tests, uric acid, electrolytes, etc.
Any ≥Grade 4 infusion-related reactions (IRRs) or Grade 3 IRRs that do not resolve to 5 Grade 2 within 24 hours with infusion interruption, infusion rate reduction, and/or standard supportive measures. In the event of a Grade 3 IRR in ≥20% of subjects, all subsequent subjects will require premedication and/or modification of infusion approach per the recommendation of the SMC. For subjects receiving premedication, any ≥Grade 3 IRR will be considered a DLT.
Dose delay ≥14 days due to toxicity

A subject's study treatment may be discontinued for any of the following reasons: Completed treatment per protocol
AE
Pregnancy
Investigator decision
Subject decision, non-AE
   Note: Ensure that subjects who decide to stop treatment because of an AE are not included in this rationale.
Study termination by sponsor
Other, non-AE

Any subject may be discontinued from the study for any of the following reasons:
Completed study per protocol
Subject withdrawal of consent
Study termination by sponsor
Lost to follow-up
Death
Other

### Concomitant Therapy

All concomitant medications, blood products, and radiotherapy administered will be recorded from Day 1 (predose) through the safety reporting period. Any concomitant medication given for a study protocol-related AE should be recorded from the time of informed consent.

Agents containing strong CYP3A4 or P-gp inhibitors are excluded. Use of experimental antiretroviral agents is also excluded. Subjects taking any excluded ART regimen must switch to a different regimen at least 7 days prior to Day 1. Changes to ART may be made at the discretion of the investigator or infectious disease specialist if medically necessary (toxicity, failure of regimen, etc.). Subjects must remain on a permitted ART regimen while receiving brentuximab vedotin.

Subjects with hepatitis B must be on anti-hepatitis B therapy.

The use of platelet and/or red blood cell supportive growth factors or transfusions when applicable is allowed. The use of colony stimulating factors for the treatment of neutropenia per institutional practice is permitted during therapy. Concomitant prednisone (or equivalent) may be used at a dose of ≤20 mg/day.

Routine premedication for infusion reactions should not be administered prior to the first dose of brentuximab vedotin. However, subjects who experience a Grade 1 or Grade 2 IRR may receive subsequent treatment with premedication.

Subjects may not receive other investigational drugs, immunomodulating therapy (excluding premedication steroid), or systemic chemotherapy within 4 weeks prior to the Screening Visit. Zidovudine and didanosine are excluded.

MMAE is primarily metabolized by CYP3A4 and co-administration of brentuximab vedotin with strong CYP3A4 inhibitors may therefore increase exposure to MMAE. Co-administration of brentuximab vedotin with P-gp inhibitors also may increase exposure to MMAE. Use of agents containing strong CYP3A4 or P-gp inhibitors, or experimental antiretrovirals is prohibited in this study.

Attenuated vaccines are prohibited within 2 weeks prior to the first dose of study drug and throughout the Treatment Period.

### Study Assessments

Only subjects who meet all inclusion and exclusion criteria will be enrolled in this study. Subject medical history includes a thorough review of significant past medical history, current conditions, any treatment for prior malignancies and response to prior treatment, and any concomitant medications. Physical examinations should include assessments of the following body parts/systems: abdomen, extremities, head, heart, lungs, neck, and neurological. Weight and height will also be measured. Blood sampling will include a serum chemistry panel (fasting glucose required at baseline only) and CBC with differential, in addition to HgbAlc, Hepatitis B and C serologies, and serum or urine β-hCG pregnancy test for subjects of childbearing potential. CD4⁺ and CD8⁺ T-cell count will be assessed at Screening. Blood samples will also be collected to assess HIV viral load. An ECG will be performed at Screening. Subjects will complete a patient-reported outcomes (PRO) assessment (WHOQOL-HIV BREF) at Baseline.

The determination of the effect of brentuximab vedotin in this subject population will be based on CD4⁺ T-cell lymphocyte count at Week 16 and throughout the duration of the study. Effect will also be determined by CD8⁺ T-cell lymphocyte count, CD4:CD8 ratio, T-cell subsets, HIV viral load, and HIV-1 viral persistence.

Sensitive, qualified assays will be used to measure drug analytes including brentuximab vedotin ADC and MMAE concentrations in serum or plasma. Blood samples for PK testing will be collected at predose on Day 1 and at Weeks 2, 4, 6, 8, 10, and 16 and at the end of infusion on Day 1. Select PK parameters to be evaluated include concentration at the end of infusion (C_{coi}) and trough concentration (C_{trough}). The incidence of ADA to brentuximab vedotin will also be assessed.

Blood samples will be collected at baseline Weeks 2, 4, 6, 8, 16, 24, 32, and 48, for evaluation of treatment-induced immunological and molecular changes, which may include CD4⁺ T cells, CD8⁺ T cells, Treg, and other immune subsets, cell-surface expression of CD30, soluble CD30, T-cell function, and circulating cytokine/chemokine and other mediators. The effect of brentuximab vedotin on CD4⁺ and CD8⁺ T-cell reconstitution will be assessed. The effect of brentuximab vedotin on HIV viral load and measures of viral persistence, such as cell-associated and plasma HIV-1 RNA and DNA will also be evaluated. The potential association between CD30 expression and/or soluble CD30 and brentuximab vedotin-induced immune reconstitution will also be assessed. Assays may include but may not be limited to flow cytometry, PCR, enzyme-linked immunosorbent assay (ELISA), Luminex, enzyme-linked immunospot (ELISpot), and mass cytometry.

Samples will be drawn for local labs. Local laboratory testing will include institutional standard tests for evaluating safety and making clinical decisions. The following laboratory assessments will be performed by the local laboratories to evaluate safety at scheduled timepoints during the course of the study:
The chemistry panel is to include the following tests: albumin, alkaline phosphatase, ALT, AST, blood urea nitrogen, calcium, creatinine, chloride, glucose, lactate dehydrogenase, phosphorus, potassium, sodium, total bilirubin, amylase, lipase, and uric acid.
The CBC with differential is to include the following tests: white blood cell count with five-part differential (neutrophils, lymphocytes, monocytes, eosinophils, and basophils), platelet count, hemoglobin, and hematocrit.
HgbAlc
The estimated GFR should be calculated using the MDRD equation as applicable, with serum creatinine (Scr) reported in mg/dL.

GFR (mL/min/1.73 m²)=175×(Scr)^{-1.154}×(Age)^{-0.203}×(0.742 if female)×(1.212 if African American)
A serum or urine β-hCG pregnancy test for subjects of childbearing potential.
Hepatitis B and C serologies
   If hepatitis C serology is positive, HCV RNA test by PCR is required to confirm

### Data Analysis Methods

The study is designed to estimate the treatment difference (A, treatment arm—control arm) in the primary endpoint (the proportion of subjects at Week 16 with CD4⁺ T-cell lymphocyte count >200 cells/μL, with a minimum increase of 50 cells/μL) at a reasonable level of precision. Approximately 60 subjects will be enrolled in this study, with 40 subjects in the treatment arm (Arm 1) and 20 in the control arm (Arm 2). Assuming the observed proportion in the treatment arm (πτ) is 60%, and the observed proportion in the control arm (ae) is 10%, the point estimate of treatment difference will be 50%, with the two-sided 95% confidence interval of (30%, 70%), based on normal approximation. Additional possible scenarios and the associated 95% confidence intervals are included in Table 6.

TABLE 6
Sample size determination table

| | | | Two-sided 95% CI for Δ based |
|---|---|---|---|
| π_{T} | π_{c} | Δ | on normal approximation |
| 60% | 10% | 50% | (30%, 70%) |
| 80% | 10% | 70% | (52%, 88%) |
| 60% | 20% | 40% | (17%, 63%) |
| 80% | 20% | 60% | (39%, 82%) |

What is claimed is:

1. A method of increasing CD4⁺ T-cell lymphocyte count in a subject infected with human immunodeficiency virus (HIV) comprising administering to the subject an antibody-drug conjugate, wherein the antibody-drug conjugate comprises an anti-CD30 antibody or an antigen-binding portion thereof conjugated to a monomethyl auristatin.

2. The method of claim 1, wherein the HIV infection is an HIV-1 infection.

3. The method of claim 1 or claim 2, wherein the subject has a CD4⁺ T-cell lymphocyte count of <200 cells/μL prior to administration of the antibody-drug conjugate.

4. The method of any one of claims 1-3, wherein the subject has a CD4⁺ T-cell lymphocyte count of ≥50 cells/μL prior to administration of the antibody-drug conjugate.

5. The method of any one of claims 1-4, wherein the subject has had a plasma HIV viral load 5 50 copies/mL for at least 6 months prior to administration of the antibody-drug conjugate.

6. The method of any one of claims 1-4, wherein the subject has had a plasma HIV viral load <50 copies/mL for at least 12 months prior to administration of the antibody-drug conjugate.

7. The method of any one of claims 1-4, wherein the subject has had a plasma HIV viral load <50 copies/mL for at least 24 months prior to administration of the antibody-drug conjugate.

8. The method of any one of claims 1-7, wherein the subject does not have a hematologic cancer at the time of administration of the antibody-drug conjugate.

9. The method of any one of claims 1-7, wherein the subject has not had a hematologic cancer for at least 12 months prior to the administration of the antibody-drug conjugate.

10. The method of any one of claims 1-7, wherein the subject has not had a hematologic cancer for at least 24 months prior to the administration of the antibody-drug conjugate.

11. The method of any one of claims 8-10, wherein the hematologic cancer is selected from the group consisting of classical Hodgkin Lymphoma, non-Hodgkin Lymphoma, cutaneous T-cell lymphoma (CTCL), and anaplastic large cell lymphoma (ALCL).

12. The method of claim 11, wherein the hematologic cancer is classical Hodgkin Lymphoma.

13. The method of claim 12, wherein the classical Hodgkin Lymphoma is a stage IIA with bulky disease, stage IIB, stage III or stage IV classical Hodgkin Lymphoma.

14. The method of claim 11, wherein the anaplastic large cell lymphoma (ALCL) is a systemic anaplastic large cell lymphoma (sALCL).

15. The method of claim 11, wherein the anaplastic large cell lymphoma (ALCL) is a primary cutaneous anaplastic large cell lymphoma (pcALCL).

16. The method of claim 11, wherein the cutaneous T-cell lymphoma (CTCL) is a mycosis fungoides (MF).

17. The method of claim 16, wherein the mycosis fungoides (MF) is a CD30-positive mycosis fungoides (MF).

18. The method of any one of claims 1-17, wherein the anti-CD30 antibody of the antibody-drug conjugate comprises a heavy chain variable region and a light chain variable region, wherein the heavy chain variable region comprises:
(i) a CDR-H1 comprising the amino acid sequence of SEQ ID NO: 1;
(ii) a CDR-H2 comprising the amino acid sequence of SEQ ID NO: 2; and
(iii) a CDR-H3 comprising the amino acid sequence of SEQ ID NO: 3; and
wherein the light chain variable region comprises:
(i) a CDR-L1 comprising the amino acid sequence of SEQ ID NO: 4:
(ii) a CDR-L2 comprising the amino acid sequence of SEQ ID NO: 5; and
(iii) a CDR-L3 comprising the amino acid sequence of SEQ ID NO: 6.

19. The method of any one of claims 1-18, wherein the anti-CD30 antibody of the antibody-drug conjugate comprises a heavy chain variable region comprising an amino acid sequence at least 85% identical to the amino acid sequence of SEQ ID NO: 7 and a light chain variable region comprising an amino acid sequence at least 85% identical to the amino acid sequence of SEQ ID NO: 8.

20. The method of any one of claims 1-18, wherein the anti-CD30 antibody of the antibody-drug conjugate comprises a heavy chain variable region comprising an amino acid sequence at least 90% identical to the amino acid sequence of SEQ ID NO: 7 and a light chain variable region comprising an amino acid sequence at least 90% identical to the amino acid sequence of SEQ ID NO: 8.

21. The method of any one of claims 1-18, wherein the anti-CD30 antibody of the antibody-drug conjugate comprises a heavy chain variable region comprising an amino acid sequence at least 95% identical to the amino acid sequence of SEQ ID NO: 7 and a light chain variable region comprising an amino acid sequence at least 95% identical to the amino acid sequence of SEQ ID NO: 8.

22. The method of any one of claims 1-18, wherein the anti-CD30 antibody of the antibody-drug conjugate comprises a heavy chain variable region comprising the amino acid sequence of SEQ ID NO: 7 and a light chain variable region comprising the amino acid sequence of SEQ ID NO: 8.

23. The method of any one of claims 1-18, wherein the anti-CD30 antibody is AC10.

24. The method of any one of claims 1-18, wherein the anti-CD30 antibody is cAC10.

25. The method of any one of claims 1-24, wherein the antibody-drug conjugate further comprises a linker between the anti-CD30 antibody or antigen-binding portion thereof and the monomethyl auristatin.

26. The method of claim 25, wherein the linker is a cleavable peptide linker.

27. The method of claim 26, wherein the cleavable peptide linker has a formula: -MC-vc-PAB-.

28. The method of any one of claims 1-27, wherein the monomethyl auristatin is monomethyl auristatin E (MMAE).

29. The method of any one of claims 1-27, wherein the monomethyl auristatin is monomethyl auristatin F (MMAF).

30. The method of any one of claims 1-18, wherein the antibody-drug conjugate is brentuximab vedotin.

31. The method of any one of claims 1-30, wherein the antibody-drug conjugate is administered at a dose ranging from of about 1.2 mg/kg of the subject's body weight.

32. The method of any one of claims 1-30, wherein the antibody-drug conjugate is administered at a dose ranging from of 1.2 mg/kg of the subject's body weight.

33. The method of any one of claims 1-30, wherein the antibody-drug conjugate is administered at a dose of about 0.9 mg/kg of the subject's body weight.

34. The method of any one of claims 1-30, wherein the antibody-drug conjugate is administered at a dose of 0.9 mg/kg of the subject's body weight.

35. The method of any one of claims 1-34, wherein the antibody-drug conjugate is administered once about every 2 weeks.

36. The method of any one of claims 1-34, wherein the antibody-drug conjugate is administered once every 2 weeks.

37. The method of claim 35 or 36, wherein the antibody-drug conjugate is administered for four 2-week treatment cycles.

38. The method of any one of claims 1-37, wherein the antibody-drug conjugate is administered to the subject by intravenous infusion.

39. The method of claim 38, wherein the intravenous infusion is about a 30 minute infusion.

40. The method of any one of claims 1-39, wherein the subject has a life expectancy of greater than 9 months prior to administration of the antibody-drug conjugate.

41. The method of any one of claims 1-40, wherein the subject has received antiretroviral therapy (ART) for at least 24 weeks prior to administration of the antibody-drug conjugate.

42. The method of claim 41, wherein the subject has received ART for at least 12 months prior to administration of the antibody-drug conjugate.

43. The method of claim 41, wherein the subject has received ART for at least 24 months prior to administration of the antibody-drug conjugate.

44. The method of any one of claims 1-43, wherein the antibody-drug conjugate is administered in combination with ART.

45. The method of any one of claims 41-44, wherein the ART is a nucleoside reverse transcriptase inhibitor, non-nucleoside reverse transcriptase inhibitor, protease inhibitor, fusion inhibitor, CCR5 antagonist, integrase inhibitor, post-attachment inhibitor, or pharmacokinetic enhancer.

46. The method of claim 45, wherein the ART comprises two or more of a nucleoside reverse transcriptase inhibitor, non-nucleoside reverse transcriptase inhibitor, protease inhibitor, fusion inhibitor, CCR5 antagonist, integrase inhibitor, post-attachment inhibitor, and pharmacokinetic enhancer.

47. The method of claim 45, wherein the ART comprises three or more of a nucleoside reverse transcriptase inhibitor, non-nucleoside reverse transcriptase inhibitor, protease inhibitor, fusion inhibitor, CCR5 antagonist, integrase inhibitor, post-attachment inhibitor, and pharmacokinetic enhancer.

48. The method of claim 45, wherein the ART comprises four or more of a nucleoside reverse transcriptase inhibitor, non-nucleoside reverse transcriptase inhibitor, protease inhibitor, fusion inhibitor, CCR5 antagonist, integrase inhibitor, post-attachment inhibitor, and pharmacokinetic enhancer.

49. The method of any one of claims 41-48, wherein the ART comprises one or more of abacavir, emtricitabine, lamivudine, tenofovir disoproxil fumarate, zidovudine, doravirine, efavirenz, etravirine, nevirapine, rilpivirine, atazanavir, darunavir, fosamprenavir, ritonavir, saquinavir, tipranavir, enfuvirtide, maraviroc, dolutegravir, raltegravir, ibalizumab, and cobicistat.

50. The method of any one of claims 41-49, wherein the ART does not comprise a strong CYP3A4 inhibitor.

51. The method of any one of claims 41-49, wherein the ART does not comprise a strong P-gp inhibitor.

52. The method of any one of claims 1-51, wherein administering the antibody-drug conjugate results an increase in the CD4 T-cell lymphocyte count in the subject to above 200 cells/μL.

53. The method of any one of claims 1-52, wherein administering the antibody-drug conjugate results in an increase in the CD4⁺ T-cell lymphocyte count by at least 50 cells/μL relative to the CD4⁺ T-cell lymphocyte count prior to administration.

54. The method of any one of claims 1-53, wherein administering the antibody-drug conjugate results an increase in the CD8⁺ T-cell lymphocyte count in the subject relative to the CD8⁺ T-cell lymphocyte count prior to administration.

55. The method of any one of claims 1-54, wherein administering the antibody-drug conjugate results in a decrease in the number of Treg cells relative to the number prior to the administration of the antibody-drug conjugate.

56. The method of claim 55, wherein the Treg cells are CD4⁺.

57. The method of claim 55 or claim 56, wherein the Treg cells are CD30⁺.

58. The method of any one of claims 1-57, wherein administering the antibody-drug conjugate results in a decrease in the number of memory T cells relative to the number prior to the administration of the antibody-drug conjugate.

59. The method of claim 58, wherein the memory T cells are CD4⁺.

60. The method of claim 58 or claim 59, wherein the memory T cells are CD30⁺.

61. The method of any one of claims 1-60, wherein the subject has not been administered the antibody-drug conjugate prior to the administration to increase CD4⁺ T-cell lymphocyte count in the subject.

62. The method of any one of claims 1-61, wherein the subject is a human.

63. A kit comprising:
(a) a dosage ranging from about 0.1 mg to about 500 mg of an of an antibody-drug conjugate that binds to CD30, wherein the antibody-drug conjugate comprises an anti-CD30 antibody or an antigen-binding fragment thereof conjugated to a monomethyl auristatin or a functional analog thereof or a functional derivative thereof; and
(b) instructions for using the antibody drug conjugate according to the method of any one of claims 1-62.

64. Use of an antibody-drug conjugate that binds to CD30 for the manufacture of a medicament for use in the method of any one of claims 1-62.

65. An antibody-drug conjugate that binds to CD30 for use in the method of any one of claims 1-62.


SEQUENCE LISTING (16 sequences)

SEQ ID NO: 1 - protein, 5 aa, Artificial Sequence
  Synthetic Construct
DYYIT

SEQ ID NO: 2 - protein, 17 aa, Artificial Sequence
  Synthetic Construct
WIYPGSGNTKYNEKFKG

SEQ ID NO: 3 - protein, 8 aa, Artificial Sequence
  Synthetic Construct
YGNYWFAY

SEQ ID NO: 4 - protein, 15 aa, Artificial Sequence
  Synthetic Construct
KASQSVDFDGDSYMN

SEQ ID NO: 5 - protein, 7 aa, Artificial Sequence
  Synthetic Construct
AASNLES

SEQ ID NO: 6 - protein, 9 aa, Artificial Sequence
  Synthetic Construct
QQSNEDPWT

SEQ ID NO: 7 - protein, 117 aa, Artificial Sequence
  Synthetic Construct
QIQLQQSGPEVVKPGASVKISCKASGYTFTDYYITWVKQKPGQGLEWIGWIYPGSGNTKYNEKFKGKATLTVDTSSSTAFMQLSSLTSEDTAVYFCANYGNYWFAYWGQGTQVTVSA

SEQ ID NO: 8 - protein, 111 aa, Artificial Sequence
  Synthetic Construct
DIVLTQSPASLAVSLGQRATISCKASQSVDFDGDSYMNWYQQKPGQPPKVLIYAASNLESGIPARFSGSGSGTDFTLNIHPVEEEDAATYYCQQSNEDPWTFGGGTKLEIK

SEQ ID NO: 9 - protein, 30 aa, Artificial Sequence
  Synthetic Construct
QIQLQQSGPEVVKPGASVKISCKASGYTFT

SEQ ID NO: 10 - protein, 14 aa, Artificial Sequence
  Synthetic Construct
WVKQKPGQGLEWIG

SEQ ID NO: 11 - protein, 32 aa, Artificial Sequence
  Synthetic Construct
KATLTVDTSSSTAFMQLSSLTSEDTAVYFCAN

SEQ ID NO: 12 - protein, 11 aa, Artificial Sequence
  Synthetic Construct
WGQGTQVTVSA

SEQ ID NO: 13 - protein, 23 aa, Artificial Sequence
  Synthetic Construct
DIVLTQSPASLAVSLGQRATISC

SEQ ID NO: 14 - protein, 15 aa, Artificial Sequence
  Synthetic Construct
WYQQKPGQPPKVLIY

SEQ ID NO: 15 - protein, 32 aa, Artificial Sequence
  Synthetic Construct
GIPARFSGSGSGTDFTLNIHPVEEEDAATYYC

SEQ ID NO: 16 - protein, 10 aa, Artificial Sequence
  Synthetic Construct
FGGGTKLEIK